SEQUENCE LISTING (78 sequences)

SEQ ID NO: 1 - protein, 736 aa, Artificial Sequence
  adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 736 aa, Artificial Sequence
  AAV isolate
MTADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKQLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTGGNFHPSPLMGGFGMKHPPPQILIKNTPVPADPPTAFNKDKLNSFITQYSTGQVSVEIEWELQKENSKRWNPEIQYTSNYYKSNNVEFAVNTGGVYSEPRPIGTRYLTRNL

SEQ ID NO: 4 - protein, 736 aa, Artificial Sequence
  AAV isolate
MAADGYLPDWLEDNLSEGIREWWALKPGAPQPKANQQHQDNARGLVLPGYKYLGPGNGLDKGEPINAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTYGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIDKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 6 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVLQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGSSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQARAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAVDAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPREPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASCGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDRAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKKNSKRWNPEIQYTSNYYKSNNVEFAVNTEGVYSEPRPIGTRYLTRNL

SEQ ID NO: 12 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPHDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFNVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSMEIEWELQKENSKRWNPEIQYTSNYYKSNNVEFAVNTEGVYSEPRPIGTRYLTRNL

SEQ ID NO: 13 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - DNA, 145 bp, Artificial Sequence
  Synthetic nucleic acid sequence
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 15 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWERQKENSKRWNPEIQYTSNYYKSNNVEFAVNTEGVYSEPRPIGTRYLTRNL

SEQ ID NO: 16 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFADSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEIAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYCKSNNVEFAVNTEGVYSEPRPIGTRYLTRNL

SEQ ID NO: 18 - DNA, 145 bp, Artificial Sequence
  AAV2 5′ ITR
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 19 - DNA, 145 bp, Artificial Sequence
  Synthetic nucleic acid sequence
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 20 - DNA, 167 bp, Artificial Sequence
  AAV5 5′ ITR
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgta

SEQ ID NO: 21 - DNA, 167 bp, Artificial Sequence
  AAV5 3′ ITR
tacaaaacctccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacaggggggagag

SEQ ID NO: 22 - protein, 621 aa, Artificial Sequence
  AAV2 Rep
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 23 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 24 - DNA, 1653 bp, Homo sapiens
atgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttga

SEQ ID NO: 25 - DNA, 1735 bp, Artificial Sequence
  Synthetic nucleic acid sequence
atgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggtaagggtttaagggatggttggttggtggggtattaatgtttaattacctggagcacctgcctgaaatcactttttttcaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttga

SEQ ID NO: 26 - DNA, 1653 bp, Artificial Sequence
  Synthetic nucleic acid sequence
atgcccccacccaggaccggaagaggcctgctgtggctgggcctggtgctctcttccgtgtgcgtggccctgggaagcgaaacccaggccaacagcacaaccgacgccctgaatgtgctgctgatcattgtggacgatctgagaccctccctgggctgttacggcgacaaactggtgcggtccccaaacatcgaccagctggcctcccactccctgctgttccagaacgccttcgcccagcaggccgtgtgtgcccccagcagggtgagcttcctgaccggcagaagacctgacaccaccaggctgtacgactttaacagctactggcgggtgcacgccggcaatttcagcaccattcctcagtacttcaaggagaatggctacgtgacaatgtccgtgggcaaggtgtttcatcccggcattagctccaaccacaccgacgatagcccatactcctggtccttccccccctaccatccctccagcgagaagtacgagaacaccaaaacctgcagaggccctgacggagagctgcacgccaacctgctgtgccctgtggatgtcctggatgtgcccgaaggcaccctgccagacaagcagtccacagagcaggccatccagctgctggagaagatgaagacaagcgccagccccttctttctggccgtgggataccacaagcctcacattccattccggtacccaaaagagttccagaagctgtaccctctggaaaacatcaccctggcccctgaccccgaggtgccagacgggctgcctcctgtggcctacaatccctggatggacatcagacagcgggaggatgtgcaggccctgaatatttccgtgccctatgggcccatccctgtggactttcagcggaaaatcagacagtcttactttgccagcgtgtcctacctggacacccaggtgggccgcctgctctcagccctggacgacctgcagctggccaattccaccatcatcgccttcaccagcgatcacggctgggccctgggcgagcacggggagtgggccaaatacagcaacttcgatgtggccacccacgtgcctctgattttttatgtgcccggccggacagccagcctgcccgaggccggggagaagctctttccttacctggaccctttcgactctgccagccagctgatggaacctggcagacagagcatggacctggtggagctggtgagcctcttccccactctggccggcctggctggcctgcaggtgccaccaagatgcccagtgccttctttccacgtggagctgtgtagagagggaaagaacctgctgaagcacttcagatttagagatctggaggaggatccctacctgccaggcaacccaagggagctgatcgcctacagccagtatcccagaccctctgatatcccccagtggaacagcgataagccctccctgaaagacatcaagattatgggctactccatcaggaccattgactaccggtacacagtgtgggtgggcttcaaccccgatgagtttctggccaacttcagcgacatccacgccggcgagctgtattttgtggactccgaccccctgcaggaccacaacatgtacaacgactcccagggcggcgacctgttccagctgctgatgccctga

SEQ ID NO: 27 - DNA, 1735 bp, Artificial Sequence
  Synthetic nucleic acid sequence
atgcccccacccaggaccggaagaggcctgctgtggctgggcctggtgctctcttccgtgtgcgtggccctgggaagcgaaacccaggccaacagcacaaccgacgccctgaatgtgctgctgatcattgtggacgatctgagaccctccctgggctgttacggcgacaaactggtgcggtccccaaacatcgaccagctggcctcccactccctgctgttccagaacgccttcgcccagcaggccgtgtgtgcccccagcagggtgagcttcctgaccggcagaagacctgacaccaccaggctgtacgactttaacagctactggcgggtgcacgccggcaatttcagcaccattcctcagtacttcaaggagaatggctacgtgacaatgtccgtgggcaaggtgtttcatcccggcattagctccaaccacaccgacgatagcccatactcctggtccttccccccctaccatccctccagcgagaagtacgagaacaccaaaacctgcagaggccctgacggagagctgcacgccaacctgctgtgccctgtggatgtcctggatgtgcccgaaggtaagggtttaagggatggttggttggtggggtattaatgtttaattacctggagcacctgcctgaaatcactttttttcaggcaccctgccagacaagcagtccacagagcaggccatccagctgctggagaagatgaagacaagcgccagccccttctttctggccgtgggataccacaagcctcacattccattccggtacccaaaagagttccagaagctgtaccctctggaaaacatcaccctggcccctgaccccgaggtgccagacgggctgcctcctgtggcctacaatccctggatggacatcagacagcgggaggatgtgcaggccctgaatatttccgtgccctatgggcccatccctgtggactttcagcggaaaatcagacagtcttactttgccagcgtgtcctacctggacacccaggtgggccgcctgctctcagccctggacgacctgcagctggccaattccaccatcatcgccttcaccagcgatcacggctgggccctgggcgagcacggggagtgggccaaatacagcaacttcgatgtggccacccacgtgcctctgattttttatgtgcccggccggacagccagcctgcccgaggccggggagaagctctttccttacctggaccctttcgactctgccagccagctgatggaacctggcagacagagcatggacctggtggagctggtgagcctcttccccactctggccggcctggctggcctgcaggtgccaccaagatgcccagtgccttctttccacgtggagctgtgtagagagggaaagaacctgctgaagcacttcagatttagagatctggaggaggatccctacctgccaggcaacccaagggagctgatcgcctacagccagtatcccagaccctctgatatcccccagtggaacagcgataagccctccctgaaagacatcaagattatgggctactccatcaggaccattgactaccggtacacagtgtgggtgggcttcaaccccgatgagtttctggccaacttcagcgacatccacgccggcgagctgtattttgtggactccgaccccctgcaggaccacaacatgtacaacgactcccagggcggcgacctgttccagctgctgatgccctga

SEQ ID NO: 28 - DNA, 182 bp, Artificial Sequence
  Synthetic nucleic acid sequence
gtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 29 - DNA, 173 bp, Artificial Sequence
  Synthetic nucleic acid sequence
actcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgt

SEQ ID NO: 30 - DNA, 201 bp, Artificial Sequence
  Synthetic nucleic acid sequence
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtgatcatcacttgtga

SEQ ID NO: 31 - DNA, 93 bp, Artificial Sequence
  Synthetic nucleic acid sequence
ctctaaggtaaatataaaatttttaagtgtataatgtgttaaactactgattctaattgtttctctcttttagattccaacctttggaactga

SEQ ID NO: 32 - DNA, 924 bp, Artificial Sequence
  Synthetic nucleic acid sequence
gtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 33 - DNA, 82 bp, Artificial Sequence
  Synthetic nucleic acid sequence
gtaagggtttaagggatggttggttggtggggtattaatgtttaattacctggagcacctgcctgaaatcactttttttcag

SEQ ID NO: 34 - DNA, 122 bp, Artificial Sequence
  Synthetic nucleic acid sequence
aacttgtttattgcagcttataatggttacaaataaagcaatagcatcacaaatttcacaaataaagcatttttttcactgcattctagttgtggtttgtccaaactcatcaatgtatctta

SEQ ID NO: 35 - DNA, 133 bp, Artificial Sequence
  Synthetic nucleic acid sequence
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 36 - DNA, 1676 bp, Artificial Sequence
  Synthetic nucleic acid sequence
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 37 - DNA, 2112 bp, Artificial Sequence
  Synthetic nucleic acid sequence
actcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaccatgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggtaagggtttaagggatggttggttggtggggtattaatgtttaattacctggagcacctgcctgaaatcactttttttcaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 38 - DNA, 2482 bp, Artificial Sequence
  Synthetic nucleic acid sequence
ctgcgcgctcgctcgctcactgaggccgcccgggcaaagcccgggcgtcgggcgacctttggtcgcccggcctcagtgagcgagcgagcgcgcagagagggagtggggaggggtggagtcgtgacgtgaattacgtcatagggttagggaggtcctgcatatgcggccgcaactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaccatgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggtaagggtttaagggatggttggttggtggggtattaatgtttaattacctggagcacctgcctgaaatcactttttttcaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 39 - DNA, 1168 bp, Artificial Sequence
  Synthetic nucleic acid sequence
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 40 - DNA, 100 bp, Artificial Sequence
  Synthetic nucleic acid sequence
ccaaaatcaacgggactttccaaaatgtcgtaataaccccgccccgttgacgcaaatgggcggtaggcgtgtacggtgggaggtctatataagcagagct

SEQ ID NO: 41 - DNA, 95 bp, Artificial Sequence
  Synthetic nucleic acid sequence
cctctgctaaccatgttcatgccttcttctttttcctacagctcctgggcaacgtgctggttattgtgctgtctcatcattttggcaaagaattc

SEQ ID NO: 42 - DNA, 1873 bp, Artificial Sequence
  Synthetic nucleic acid sequence
gatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 43 - DNA, 2113 bp, Artificial Sequence
  Synthetic nucleic acid sequence
aactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaccatgcccccacccaggaccggaagaggcctgctgtggctgggcctggtgctctcttccgtgtgcgtggccctgggaagcgaaacccaggccaacagcacaaccgacgccctgaatgtgctgctgatcattgtggacgatctgagaccctccctgggctgttacggcgacaaactggtgcggtccccaaacatcgaccagctggcctcccactccctgctgttccagaacgccttcgcccagcaggccgtgtgtgcccccagcagggtgagcttcctgaccggcagaagacctgacaccaccaggctgtacgactttaacagctactggcgggtgcacgccggcaatttcagcaccattcctcagtacttcaaggagaatggctacgtgacaatgtccgtgggcaaggtgtttcatcccggcattagctccaaccacaccgacgatagcccatactcctggtccttccccccctaccatccctccagcgagaagtacgagaacaccaaaacctgcagaggccctgacggagagctgcacgccaacctgctgtgccctgtggatgtcctggatgtgcccgaaggtaagggtttaagggatggttggttggtggggtattaatgtttaattacctggagcacctgcctgaaatcactttttttcaggcaccctgccagacaagcagtccacagagcaggccatccagctgctggagaagatgaagacaagcgccagccccttctttctggccgtgggataccacaagcctcacattccattccggtacccaaaagagttccagaagctgtaccctctggaaaacatcaccctggcccctgaccccgaggtgccagacgggctgcctcctgtggcctacaatccctggatggacatcagacagcgggaggatgtgcaggccctgaatatttccgtgccctatgggcccatccctgtggactttcagcggaaaatcagacagtcttactttgccagcgtgtcctacctggacacccaggtgggccgcctgctctcagccctggacgacctgcagctggccaattccaccatcatcgccttcaccagcgatcacggctgggccctgggcgagcacggggagtgggccaaatacagcaacttcgatgtggccacccacgtgcctctgattttttatgtgcccggccggacagccagcctgcccgaggccggggagaagctctttccttacctggaccctttcgactctgccagccagctgatggaacctggcagacagagcatggacctggtggagctggtgagcctcttccccactctggccggcctggctggcctgcaggtgccaccaagatgcccagtgccttctttccacgtggagctgtgtagagagggaaagaacctgctgaagcacttcagatttagagatctggaggaggatccctacctgccaggcaacccaagggagctgatcgcctacagccagtatcccagaccctctgatatcccccagtggaacagcgataagccctccctgaaagacatcaagattatgggctactccatcaggaccattgactaccggtacacagtgtgggtgggcttcaaccccgatgagtttctggccaacttcagcgacatccacgccggcgagctgtattttgtggactccgaccccctgcaggaccacaacatgtacaacgactcccagggcggcgacctgttccagctgctgatgccctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 44 - DNA, 913 bp, Artificial Sequence
  Synthetic nucleic acid sequence
ggcatcctaaaaaatattcagtggaaacgtaaaaacattaaagactgattaaacatcgcagcatgacacagatttagcaactgagcataaataatttgactcggatactgctccaaaatccgaagaggaccaatttcttccaggaggacaactacctcgtcctctgcagacccctctcctcggcagctgaaggagtgtggccaatctgcctccacctccccgcggaccccctactctcaggacctcctgcagcaccccaaactggaagtggccgctgcagacccaaggacgaggggcacgcgggagccggcagccctagtggagcggttggagatgttgaggtgggagggtcacccaggtggggtgaggctggggtaggtagcggagtgaacggcttccgaagctctgggccgcccccaggttggactaagcaggcgctctgtcttcgcccccgcccagggtgggcgtctcctgaggactccccgccacacctgacccgagaccgcgcgcccagcctagaacgcttccccgacccagcgtagggccgccgcgactggcgggcgagggtcggcgggaggcctggcgaacccgggggcgggaccaggcgggcaaggcccggctgccgcagcgccgctctgcgcgaggcggctccgccgcggcggagggatacggcgcaccatatatatatcgcggggcgcagactcgcgctccggcagtggtgctgggagtgtcgtggacgccgtgccgttactcgtagtcaggcggcggcgcaggcggcggcggcggcatagcgcacagcgcgccttagcagcagcagcagcagcagcggcatcggaggtacccccgccgtcgcagcccccgcgctggtgcagccaccctcgctccctctgctcttcctcccttcgctcgcacc

SEQ ID NO: 45 - DNA, 198 bp, Artificial Sequence
  Synthetic nucleic acid sequence
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 46 - DNA, 380 bp, Artificial Sequence
  Synthetic nucleic acid sequence
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

SEQ ID NO: 47 - DNA, 1246 bp, Artificial Sequence
  Synthetic nucleic acid sequence
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcgg

SEQ ID NO: 48 - DNA, 1061 bp, Artificial Sequence
  Synthetic nucleic acid sequence
tagggaggtcctgcacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacag

SEQ ID NO: 49 - DNA, 106 bp, Artificial Sequence
  Synthetic nucleic acid sequence
ctgcgcgctcgctcgctcactgaggccgcccgggcaaagcccgggcgtcgggcgacctttggtcgcccggcctcagtgagcgagcgagcgcgcagagagggagtgg

SEQ ID NO: 50 - DNA, 2482 bp, Artificial Sequence
  Synthetic nucleic acid sequence
ctgcgcgctcgctcgctcactgaggccgcccgggcaaagcccgggcgtcgggcgacctttggtcgcccggcctcagtgagcgagcgagcgcgcagagagggagtggggaggggtggagtcgtgacgtgaattacgtcatagggttagggaggtcctgcatatgcggccgcaactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaccatgcccccacccaggaccggaagaggcctgctgtggctgggcctggtgctctcttccgtgtgcgtggccctgggaagcgaaacccaggccaacagcacaaccgacgccctgaatgtgctgctgatcattgtggacgatctgagaccctccctgggctgttacggcgacaaactggtgcggtccccaaacatcgaccagctggcctcccactccctgctgttccagaacgccttcgcccagcaggccgtgtgtgcccccagcagggtgagcttcctgaccggcagaagacctgacaccaccaggctgtacgactttaacagctactggcgggtgcacgccggcaatttcagcaccattcctcagtacttcaaggagaatggctacgtgacaatgtccgtgggcaaggtgtttcatcccggcattagctccaaccacaccgacgatagcccatactcctggtccttccccccctaccatccctccagcgagaagtacgagaacaccaaaacctgcagaggccctgacggagagctgcacgccaacctgctgtgccctgtggatgtcctggatgtgcccgaaggtaagggtttaagggatggttggttggtggggtattaatgtttaattacctggagcacctgcctgaaatcactttttttcaggcaccctgccagacaagcagtccacagagcaggccatccagctgctggagaagatgaagacaagcgccagccccttctttctggccgtgggataccacaagcctcacattccattccggtacccaaaagagttccagaagctgtaccctctggaaaacatcaccctggcccctgaccccgaggtgccagacgggctgcctcctgtggcctacaatccctggatggacatcagacagcgggaggatgtgcaggccctgaatatttccgtgccctatgggcccatccctgtggactttcagcggaaaatcagacagtcttactttgccagcgtgtcctacctggacacccaggtgggccgcctgctctcagccctggacgacctgcagctggccaattccaccatcatcgccttcaccagcgatcacggctgggccctgggcgagcacggggagtgggccaaatacagcaacttcgatgtggccacccacgtgcctctgattttttatgtgcccggccggacagccagcctgcccgaggccggggagaagctctttccttacctggaccctttcgactctgccagccagctgatggaacctggcagacagagcatggacctggtggagctggtgagcctcttccccactctggccggcctggctggcctgcaggtgccaccaagatgcccagtgccttctttccacgtggagctgtgtagagagggaaagaacctgctgaagcacttcagatttagagatctggaggaggatccctacctgccaggcaacccaagggagctgatcgcctacagccagtatcccagaccctctgatatcccccagtggaacagcgataagccctccctgaaagacatcaagattatgggctactccatcaggaccattgactaccggtacacagtgtgggtgggcttcaaccccgatgagtttctggccaacttcagcgacatccacgccggcgagctgtattttgtggactccgaccccctgcaggaccacaacatgtacaacgactcccagggcggcgacctgttccagctgctgatgccctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 51 - DNA, 143 bp, Artificial Sequence
  Synthetic nucleic acid sequence
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 52 - DNA, 2112 bp, Artificial Sequence; identical to SEQ ID NO: 37
  Synthetic nucleic acid sequence

SEQ ID NO: 53 - DNA, 2521 bp, Artificial Sequence
  Synthetic nucleic acid sequence
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcaactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaccatgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggtaagggtttaagggatggttggttggtggggtattaatgtttaattacctggagcacctgcctgaaatcactttttttcaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 54 - DNA, 2113 bp, Artificial Sequence; identical to SEQ ID NO: 43
  Synthetic nucleic acid sequence

SEQ ID NO: 55 - DNA, 953 bp, Artificial Sequence
  Synthetic nucleic acid sequence
aattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaag

SEQ ID NO: 56 - DNA, 37 bp, Artificial Sequence
  Synthetic nucleic acid sequence
gtagataagtagcatggcgggttaatcattaactaca

SEQ ID NO: 57 - DNA, 180 bp, Artificial Sequence
  Synthetic nucleic acid sequence
gtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 58 - DNA, 2522 bp, Artificial Sequence
  Synthetic nucleic acid sequence
tttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcaactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaccatgcccccacccaggaccggaagaggcctgctgtggctgggcctggtgctctcttccgtgtgcgtggccctgggaagcgaaacccaggccaacagcacaaccgacgccctgaatgtgctgctgatcattgtggacgatctgagaccctccctgggctgttacggcgacaaactggtgcggtccccaaacatcgaccagctggcctcccactccctgctgttccagaacgccttcgcccagcaggccgtgtgtgcccccagcagggtgagcttcctgaccggcagaagacctgacaccaccaggctgtacgactttaacagctactggcgggtgcacgccggcaatttcagcaccattcctcagtacttcaaggagaatggctacgtgacaatgtccgtgggcaaggtgtttcatcccggcattagctccaaccacaccgacgatagcccatactcctggtccttccccccctaccatccctccagcgagaagtacgagaacaccaaaacctgcagaggccctgacggagagctgcacgccaacctgctgtgccctgtggatgtcctggatgtgcccgaaggtaagggtttaagggatggttggttggtggggtattaatgtttaattacctggagcacctgcctgaaatcactttttttcaggcaccctgccagacaagcagtccacagagcaggccatccagctgctggagaagatgaagacaagcgccagccccttctttctggccgtgggataccacaagcctcacattccattccggtacccaaaagagttccagaagctgtaccctctggaaaacatcaccctggcccctgaccccgaggtgccagacgggctgcctcctgtggcctacaatccctggatggacatcagacagcgggaggatgtgcaggccctgaatatttccgtgccctatgggcccatccctgtggactttcagcggaaaatcagacagtcttactttgccagcgtgtcctacctggacacccaggtgggccgcctgctctcagccctggacgacctgcagctggccaattccaccatcatcgccttcaccagcgatcacggctgggccctgggcgagcacggggagtgggccaaatacagcaacttcgatgtggccacccacgtgcctctgattttttatgtgcccggccggacagccagcctgcccgaggccggggagaagctctttccttacctggaccctttcgactctgccagccagctgatggaacctggcagacagagcatggacctggtggagctggtgagcctcttccccactctggccggcctggctggcctgcaggtgccaccaagatgcccagtgccttctttccacgtggagctgtgtagagagggaaagaacctgctgaagcacttcagatttagagatctggaggaggatccctacctgccaggcaacccaagggagctgatcgcctacagccagtatcccagaccctctgatatcccccagtggaacagcgataagccctccctgaaagacatcaagattatgggctactccatcaggaccattgactaccggtacacagtgtgggtgggcttcaaccccgatgagtttctggccaacttcagcgacatccacgccggcgagctgtattttgtggactccgaccccctgcaggaccacaacatgtacaacgactcccagggcggcgacctgttccagctgctgatgccctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaacctgcaggtctagatacgtagataagtagcatggcgggttaatcattaactacaaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 59 - DNA, 1735 bp, Artificial Sequence
  Synthetic nucleic acid sequence
atgcccccacctagaaccggcagaggcctgctgtggctgggcctggtcctgagcagcgtttgtgtggccctgggcagcgagacacaggccaactccacaacagacgccctgaacgtgctgctgatcatcgtggacgacctgagacctagcctgggatgttacggcgacaagctggtgcggagccccaacatcgaccagctggctagccacagcctgctgttccagaacgcctttgcccagcaggctgtgtgcgccccttctagagtgtcctttctgaccggcagaaggcctgacaccacaagactgtacgacttcaactcttactggcgggtgcacgccggcaacttcagcaccatcccccagtactttaaggagaacggctacgtgaccatgagcgtgggcaaggtgttccatcctggcatcagcagcaaccacaccgacgacagcccctacagctggtccttccccccctatcacccctctagcgagaagtacgagaataccaagacctgccggggccccgatggcgaactgcacgccaacctgctctgccctgtggacgtgctggatgtgcctgagggcaccctgcctgacaagcagagcaccgagcaggccatccagctgctcgaaaagatgaaaacatctgccagccctttcttcctggccgtgggctaccacaagcctcacatcccttttcggtaccctaaggtaagggtttaagggatggttggttggtggggtattaatgtttaattacctggagcacctgcctgaaatcactttttttcaggaattccaaaaactgtaccccctcgagaacataacactggctcctgaccccgaagtgccagacggcctccctccagtggcttataacccctggatggatatccggcagcgggaagatgtccaggcccttaatatcagcgtgccatacggccctatccctgtggacttccagagaaagatcagacagagctacttcgccagcgtctcctacctggatacacaggtgggaagactgctgtctgccctcgatgatctgcagctggcaaacagcacaatcatcgccttcaccagcgaccacggctgggctctgggcgaacacggcgagtgggccaagtactccaactttgacgtggccacccacgtgccactgatcttctacgtgcccggccggaccgcctctctgccagaggccggagagaaactgtttccatatctggaccccttcgacagcgcctctcagctgatggaacctggcagacaatctatggacctggtggaactggtgtccctgttccctaccctggccggactggctggccttcaagtgcctccacggtgccccgtgccaagcttccacgttgagctgtgcagagagggaaaaaacctgctgaagcacttcagattccgggacctggaggaagatccttacctccctggaaatcctagagagctgatcgcctacagccagtaccccagacccagcgacatccctcagtggaacagcgataagccctccctgaaggacattaagatcatgggatatagcatcagaaccatcgactacagatacaccgtgtgggtcggcttcaaccccgacgagttcctggccaatttcagcgacatccacgccggagaactgtacttcgtggattctgaccctctgcaggaccacaacatgtacaacgacagccagggcggcgatctgttccagctgcttatgccttga

SEQ ID NO: 60 - DNA, 1735 bp, Artificial Sequence
  Synthetic nucleic acid sequence
atgcccccccccagaaccgggagaggcctcctgtggctgggcctggtgctgagctccgtgtgtgtggccctgggcagcgagacccaggccaacagcaccacagacgccctgaatgtgctgctgatcattgtggatgacctgaggcccagcctgggctgctacggcgacaagctggtgcgcagcccaaacattgaccagctggcctcccacagcctgctgtttcagaatgccttcgcccagcaggctgtgtgtgcccccagcagagtgtcctttctgacaggccggcggccagataccaccagactgtatgacttcaattcctattggagagtgcacgctggcaacttcagcaccatcccccagtatttcaaagagaatggctacgtgaccatgtccgtgggcaaggtgttccaccctggcatcagcagcaaccacacagacgatagcccctatagctggtctttcccaccttatcatcccagcagcgagaagtatgaaaacaccaagacctgcaggggccccgacggagaactgcacgccaacctgctgtgccccgtggacgtgctggacgtgcctgagggcaccctgcccgataagcagagcactgagcaggctatccagctgctggagaagatgaagacaagcgcctctcctttcttcctggccgtgggctaccataagcctcacatccccttcaggtaccctaaggtaagggtttaagggatggttggttggtggggtattaatgtttaattacctggagcacctgcctgaaatcactttttttcaggagttccagaagctgtatcccctggagaacatcaccctggcccccgacccagaggtgcccgatggcctgccccccgtggcctacaacccatggatggatattcgccagagggaggatgtgcaggctctgaacatcagcgtgccctacggccccatccccgtggacttccagcgcaagatccggcagagctactttgcctccgtgtcttacctggatacacaggtgggcaggctgctgtccgccctggacgatctgcagctggccaatagcaccatcatcgccttcaccagcgaccatggctgggccctgggcgagcacggagaatgggccaagtacagcaactttgacgtggccacccacgtgccacttatcttctacgtgcccggcaggaccgccagcctgcccgaggctggcgagaagctgttcccctacctcgatccttttgattctgcctcccagctgatggagcccggcaggcagagcatggacctggtggagctggtgagcctgtttccaacactggccgggctggccggcctgcaggtgccacccagatgcccagtgcccagttttcatgtggagctgtgtagggagggcaagaatctgctgaagcacttcagattcagggatctggaggaggacccatacctgcctggaaatcccagggagctgatcgcatactcccagtaccccagacctagcgacatccctcagtggaactctgataagcccagcctcaaggacattaagattatgggctacagtatcaggaccatcgactacagatacacagtgtgggtgggatttaacccagacgagttcctggccaatttctccgacatccacgccggcgagctgtacttcgtggactctgaccctctgcaggaccacaacatgtacaatgacagccagggaggagacctgttccagctgctgatgccctga

SEQ ID NO: 61 - DNA, 2112 bp, Artificial Sequence
  Synthetic nucleic acid sequence
actcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaggatgcccccacctagaaccggcagaggcctgctgtggctgggcctggtcctgagcagcgtttgtgtggccctgggcagcgagacacaggccaactccacaacagacgccctgaacgtgctgctgatcatcgtggacgacctgagacctagcctgggatgttacggcgacaagctggtgcggagccccaacatcgaccagctggctagccacagcctgctgttccagaacgcctttgcccagcaggctgtgtgcgccccttctagagtgtcctttctgaccggcagaaggcctgacaccacaagactgtacgacttcaactcttactggcgggtgcacgccggcaacttcagcaccatcccccagtactttaaggagaacggctacgtgaccatgagcgtgggcaaggtgttccatcctggcatcagcagcaaccacaccgacgacagcccctacagctggtccttccccccctatcacccctctagcgagaagtacgagaataccaagacctgccggggccccgatggcgaactgcacgccaacctgctctgccctgtggacgtgctggatgtgcctgagggcaccctgcctgacaagcagagcaccgagcaggccatccagctgctcgaaaagatgaaaacatctgccagccctttcttcctggccgtgggctaccacaagcctcacatcccttttcggtaccctaaggtaagggtttaagggatggttggttggtggggtattaatgtttaattacctggagcacctgcctgaaatcactttttttcaggaattccaaaaactgtaccccctcgagaacataacactggctcctgaccccgaagtgccagacggcctccctccagtggcttataacccctggatggatatccggcagcgggaagatgtccaggcccttaatatcagcgtgccatacggccctatccctgtggacttccagagaaagatcagacagagctacttcgccagcgtctcctacctggatacacaggtgggaagactgctgtctgccctcgatgatctgcagctggcaaacagcacaatcatcgccttcaccagcgaccacggctgggctctgggcgaacacggcgagtgggccaagtactccaactttgacgtggccacccacgtgccactgatcttctacgtgcccggccggaccgcctctctgccagaggccggagagaaactgtttccatatctggaccccttcgacagcgcctctcagctgatggaacctggcagacaatctatggacctggtggaactggtgtccctgttccctaccctggccggactggctggccttcaagtgcctccacggtgccccgtgccaagcttccacgttgagctgtgcagagagggaaaaaacctgctgaagcacttcagattccgggacctggaggaagatccttacctccctggaaatcctagagagctgatcgcctacagccagtaccccagacccagcgacatccctcagtggaacagcgataagccctccctgaaggacattaagatcatgggatatagcatcagaaccatcgactacagatacaccgtgtgggtcggcttcaaccccgacgagttcctggccaatttcagcgacatccacgccggagaactgtacttcgtggattctgaccctctgcaggaccacaacatgtacaacgacagccagggcggcgatctgttccagctgcttatgccttgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 62 - DNA, 2482 bp, Artificial Sequence
  Synthetic nucleic acid sequence
ctgcgcgctcgctcgctcactgaggccgcccgggcaaagcccgggcgtcgggcgacctttggtcgcccggcctcagtgagcgagcgagcgcgcagagagggagtggggaggggtggagtcgtgacgtgaattacgtcatagggttagggaggtcctgcatatgcggccgcaactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaggatgcccccacctagaaccggcagaggcctgctgtggctgggcctggtcctgagcagcgtttgtgtggccctgggcagcgagacacaggccaactccacaacagacgccctgaacgtgctgctgatcatcgtggacgacctgagacctagcctgggatgttacggcgacaagctggtgcggagccccaacatcgaccagctggctagccacagcctgctgttccagaacgcctttgcccagcaggctgtgtgcgccccttctagagtgtcctttctgaccggcagaaggcctgacaccacaagactgtacgacttcaactcttactggcgggtgcacgccggcaacttcagcaccatcccccagtactttaaggagaacggctacgtgaccatgagcgtgggcaaggtgttccatcctggcatcagcagcaaccacaccgacgacagcccctacagctggtccttccccccctatcacccctctagcgagaagtacgagaataccaagacctgccggggccccgatggcgaactgcacgccaacctgctctgccctgtggacgtgctggatgtgcctgagggcaccctgcctgacaagcagagcaccgagcaggccatccagctgctcgaaaagatgaaaacatctgccagccctttcttcctggccgtgggctaccacaagcctcacatcccttttcggtaccctaaggtaagggtttaagggatggttggttggtggggtattaatgtttaattacctggagcacctgcctgaaatcactttttttcaggaattccaaaaactgtaccccctcgagaacataacactggctcctgaccccgaagtgccagacggcctccctccagtggcttataacccctggatggatatccggcagcgggaagatgtccaggcccttaatatcagcgtgccatacggccctatccctgtggacttccagagaaagatcagacagagctacttcgccagcgtctcctacctggatacacaggtgggaagactgctgtctgccctcgatgatctgcagctggcaaacagcacaatcatcgccttcaccagcgaccacggctgggctctgggcgaacacggcgagtgggccaagtactccaactttgacgtggccacccacgtgccactgatcttctacgtgcccggccggaccgcctctctgccagaggccggagagaaactgtttccatatctggaccccttcgacagcgcctctcagctgatggaacctggcagacaatctatggacctggtggaactggtgtccctgttccctaccctggccggactggctggccttcaagtgcctccacggtgccccgtgccaagcttccacgttgagctgtgcagagagggaaaaaacctgctgaagcacttcagattccgggacctggaggaagatccttacctccctggaaatcctagagagctgatcgcctacagccagtaccccagacccagcgacatccctcagtggaacagcgataagccctccctgaaggacattaagatcatgggatatagcatcagaaccatcgactacagatacaccgtgtgggtcggcttcaaccccgacgagttcctggccaatttcagcgacatccacgccggagaactgtacttcgtggattctgaccctctgcaggaccacaacatgtacaacgacagccagggcggcgatctgttccagctgcttatgccttgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 63 - DNA, 2112 bp, Artificial Sequence
  Synthetic nucleic acid sequence
actcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaggatgcccccccccagaaccgggagaggcctcctgtggctgggcctggtgctgagctccgtgtgtgtggccctgggcagcgagacccaggccaacagcaccacagacgccctgaatgtgctgctgatcattgtggatgacctgaggcccagcctgggctgctacggcgacaagctggtgcgcagcccaaacattgaccagctggcctcccacagcctgctgtttcagaatgccttcgcccagcaggctgtgtgtgcccccagcagagtgtcctttctgacaggccggcggccagataccaccagactgtatgacttcaattcctattggagagtgcacgctggcaacttcagcaccatcccccagtatttcaaagagaatggctacgtgaccatgtccgtgggcaaggtgttccaccctggcatcagcagcaaccacacagacgatagcccctatagctggtctttcccaccttatcatcccagcagcgagaagtatgaaaacaccaagacctgcaggggccccgacggagaactgcacgccaacctgctgtgccccgtggacgtgctggacgtgcctgagggcaccctgcccgataagcagagcactgagcaggctatccagctgctggagaagatgaagacaagcgcctctcctttcttcctggccgtgggctaccataagcctcacatccccttcaggtaccctaaggtaagggtttaagggatggttggttggtggggtattaatgtttaattacctggagcacctgcctgaaatcactttttttcaggagttccagaagctgtatcccctggagaacatcaccctggcccccgacccagaggtgcccgatggcctgccccccgtggcctacaacccatggatggatattcgccagagggaggatgtgcaggctctgaacatcagcgtgccctacggccccatccccgtggacttccagcgcaagatccggcagagctactttgcctccgtgtcttacctggatacacaggtgggcaggctgctgtccgccctggacgatctgcagctggccaatagcaccatcatcgccttcaccagcgaccatggctgggccctgggcgagcacggagaatgggccaagtacagcaactttgacgtggccacccacgtgccacttatcttctacgtgcccggcaggaccgccagcctgcccgaggctggcgagaagctgttcccctacctcgatccttttgattctgcctcccagctgatggagcccggcaggcagagcatggacctggtggagctggtgagcctgtttccaacactggccgggctggccggcctgcaggtgccacccagatgcccagtgcccagttttcatgtggagctgtgtagggagggcaagaatctgctgaagcacttcagattcagggatctggaggaggacccatacctgcctggaaatcccagggagctgatcgcatactcccagtaccccagacctagcgacatccctcagtggaactctgataagcccagcctcaaggacattaagattatgggctacagtatcaggaccatcgactacagatacacagtgtgggtgggatttaacccagacgagttcctggccaatttctccgacatccacgccggcgagctgtacttcgtggactctgaccctctgcaggaccacaacatgtacaatgacagccagggaggagacctgttccagctgctgatgccctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 64 - DNA, 2482 bp, Artificial Sequence
  Synthetic nucleic acid sequence
ctgcgcgctcgctcgctcactgaggccgcccgggcaaagcccgggcgtcgggcgacctttggtcgcccggcctcagtgagcgagcgagcgcgcagagagggagtggggaggggtggagtcgtgacgtgaattacgtcatagggttagggaggtcctgcatatgcggccgcaactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaggatgcccccccccagaaccgggagaggcctcctgtggctgggcctggtgctgagctccgtgtgtgtggccctgggcagcgagacccaggccaacagcaccacagacgccctgaatgtgctgctgatcattgtggatgacctgaggcccagcctgggctgctacggcgacaagctggtgcgcagcccaaacattgaccagctggcctcccacagcctgctgtttcagaatgccttcgcccagcaggctgtgtgtgcccccagcagagtgtcctttctgacaggccggcggccagataccaccagactgtatgacttcaattcctattggagagtgcacgctggcaacttcagcaccatcccccagtatttcaaagagaatggctacgtgaccatgtccgtgggcaaggtgttccaccctggcatcagcagcaaccacacagacgatagcccctatagctggtctttcccaccttatcatcccagcagcgagaagtatgaaaacaccaagacctgcaggggccccgacggagaactgcacgccaacctgctgtgccccgtggacgtgctggacgtgcctgagggcaccctgcccgataagcagagcactgagcaggctatccagctgctggagaagatgaagacaagcgcctctcctttcttcctggccgtgggctaccataagcctcacatccccttcaggtaccctaaggtaagggtttaagggatggttggttggtggggtattaatgtttaattacctggagcacctgcctgaaatcactttttttcaggagttccagaagctgtatcccctggagaacatcaccctggcccccgacccagaggtgcccgatggcctgccccccgtggcctacaacccatggatggatattcgccagagggaggatgtgcaggctctgaacatcagcgtgccctacggccccatccccgtggacttccagcgcaagatccggcagagctactttgcctccgtgtcttacctggatacacaggtgggcaggctgctgtccgccctggacgatctgcagctggccaatagcaccatcatcgccttcaccagcgaccatggctgggccctgggcgagcacggagaatgggccaagtacagcaactttgacgtggccacccacgtgccacttatcttctacgtgcccggcaggaccgccagcctgcccgaggctggcgagaagctgttcccctacctcgatccttttgattctgcctcccagctgatggagcccggcaggcagagcatggacctggtggagctggtgagcctgtttccaacactggccgggctggccggcctgcaggtgccacccagatgcccagtgcccagttttcatgtggagctgtgtagggagggcaagaatctgctgaagcacttcagattcagggatctggaggaggacccatacctgcctggaaatcccagggagctgatcgcatactcccagtaccccagacctagcgacatccctcagtggaactctgataagcccagcctcaaggacattaagattatgggctacagtatcaggaccatcgactacagatacacagtgtgggtgggatttaacccagacgagttcctggccaatttctccgacatccacgccggcgagctgtacttcgtggactctgaccctctgcaggaccacaacatgtacaatgacagccagggaggagacctgttccagctgctgatgccctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 65 - DNA, 2112 bp, Artificial Sequence
  Synthetic nucleic acid sequence
actcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaggatgcccccacccaggaccggaagaggcctgctgtggctgggcctggtgctctcttccgtgtgcgtggccctgggaagcgaaacccaggccaacagcacaaccgacgccctgaatgtgctgctgatcattgtggacgatctgagaccctccctgggctgttacggcgacaaactggtgcggtccccaaacatcgaccagctggcctcccactccctgctgttccagaacgccttcgcccagcaggccgtgtgtgcccccagcagggtgagcttcctgaccggcagaagacctgacaccaccaggctgtacgactttaacagctactggcgggtgcacgccggcaatttcagcaccattcctcagtacttcaaggagaatggctacgtgacaatgtccgtgggcaaggtgtttcatcccggcattagctccaaccacaccgacgatagcccatactcctggtccttccccccctaccatccctccagcgagaagtacgagaacaccaaaacctgcagaggccctgacggagagctgcacgccaacctgctgtgccctgtggatgtcctggatgtgcccgaaggtaagggtttaagggatggttggttggtggggtattaatgtttaattacctggagcacctgcctgaaatcactttttttcaggcaccctgccagacaagcagtccacagagcaggccatccagctgctggagaagatgaagacaagcgccagccccttctttctggccgtgggataccacaagcctcacattccattccggtacccaaaagagttccagaagctgtaccctctggaaaacatcaccctggcccctgaccccgaggtgccagacgggctgcctcctgtggcctacaatccctggatggacatcagacagcgggaggatgtgcaggccctgaatatttccgtgccctatgggcccatccctgtggactttcagcggaaaatcagacagtcttactttgccagcgtgtcctacctggacacccaggtgggccgcctgctctcagccctggacgacctgcagctggccaattccaccatcatcgccttcaccagcgatcacggctgggccctgggcgagcacggggagtgggccaaatacagcaacttcgatgtggccacccacgtgcctctgattttttatgtgcccggccggacagccagcctgcccgaggccggggagaagctctttccttacctggaccctttcgactctgccagccagctgatggaacctggcagacagagcatggacctggtggagctggtgagcctcttccccactctggccggcctggctggcctgcaggtgccaccaagatgcccagtgccttctttccacgtggagctgtgtagagagggaaagaacctgctgaagcacttcagatttagagatctggaggaggatccctacctgccaggcaacccaagggagctgatcgcctacagccagtatcccagaccctctgatatcccccagtggaacagcgataagccctccctgaaagacatcaagattatgggctactccatcaggaccattgactaccggtacacagtgtgggtgggcttcaaccccgatgagtttctggccaacttcagcgacatccacgccggcgagctgtattttgtggactccgaccccctgcaggaccacaacatgtacaacgactcccagggcggcgacctgttccagctgctgatgccctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 66 - DNA, 2482 bp, Artificial Sequence
  Synthetic nucleic acid sequence
ctgcgcgctcgctcgctcactgaggccgcccgggcaaagcccgggcgtcgggcgacctttggtcgcccggcctcagtgagcgagcgagcgcgcagagagggagtggggaggggtggagtcgtgacgtgaattacgtcatagggttagggaggtcctgcatatgcggccgcaactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaggatgcccccacccaggaccggaagaggcctgctgtggctgggcctggtgctctcttccgtgtgcgtggccctgggaagcgaaacccaggccaacagcacaaccgacgccctgaatgtgctgctgatcattgtggacgatctgagaccctccctgggctgttacggcgacaaactggtgcggtccccaaacatcgaccagctggcctcccactccctgctgttccagaacgccttcgcccagcaggccgtgtgtgcccccagcagggtgagcttcctgaccggcagaagacctgacaccaccaggctgtacgactttaacagctactggcgggtgcacgccggcaatttcagcaccattcctcagtacttcaaggagaatggctacgtgacaatgtccgtgggcaaggtgtttcatcccggcattagctccaaccacaccgacgatagcccatactcctggtccttccccccctaccatccctccagcgagaagtacgagaacaccaaaacctgcagaggccctgacggagagctgcacgccaacctgctgtgccctgtggatgtcctggatgtgcccgaaggtaagggtttaagggatggttggttggtggggtattaatgtttaattacctggagcacctgcctgaaatcactttttttcaggcaccctgccagacaagcagtccacagagcaggccatccagctgctggagaagatgaagacaagcgccagccccttctttctggccgtgggataccacaagcctcacattccattccggtacccaaaagagttccagaagctgtaccctctggaaaacatcaccctggcccctgaccccgaggtgccagacgggctgcctcctgtggcctacaatccctggatggacatcagacagcgggaggatgtgcaggccctgaatatttccgtgccctatgggcccatccctgtggactttcagcggaaaatcagacagtcttactttgccagcgtgtcctacctggacacccaggtgggccgcctgctctcagccctggacgacctgcagctggccaattccaccatcatcgccttcaccagcgatcacggctgggccctgggcgagcacggggagtgggccaaatacagcaacttcgatgtggccacccacgtgcctctgattttttatgtgcccggccggacagccagcctgcccgaggccggggagaagctctttccttacctggaccctttcgactctgccagccagctgatggaacctggcagacagagcatggacctggtggagctggtgagcctcttccccactctggccggcctggctggcctgcaggtgccaccaagatgcccagtgccttctttccacgtggagctgtgtagagagggaaagaacctgctgaagcacttcagatttagagatctggaggaggatccctacctgccaggcaacccaagggagctgatcgcctacagccagtatcccagaccctctgatatcccccagtggaacagcgataagccctccctgaaagacatcaagattatgggctactccatcaggaccattgactaccggtacacagtgtgggtgggcttcaaccccgatgagtttctggccaacttcagcgacatccacgccggcgagctgtattttgtggactccgaccccctgcaggaccacaacatgtacaacgactcccagggcggcgacctgttccagctgctgatgccctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 67 - DNA, 1653 bp, Artificial Sequence
  Synthetic nucleic acid sequence
atgcccccacctagaaccggcagaggcctgctgtggctgggcctggtcctgagcagcgtttgtgtggccctgggcagcgagacacaggccaactccacaacagacgccctgaacgtgctgctgatcatcgtggacgacctgagacctagcctgggatgttacggcgacaagctggtgcggagccccaacatcgaccagctggctagccacagcctgctgttccagaacgcctttgcccagcaggctgtgtgcgccccttctagagtgtcctttctgaccggcagaaggcctgacaccacaagactgtacgacttcaactcttactggcgggtgcacgccggcaacttcagcaccatcccccagtactttaaggagaacggctacgtgaccatgagcgtgggcaaggtgttccatcctggcatcagcagcaaccacaccgacgacagcccctacagctggtccttccccccctatcacccctctagcgagaagtacgagaataccaagacctgccggggccccgatggcgaactgcacgccaacctgctctgccctgtggacgtgctggatgtgcctgagggcaccctgcctgacaagcagagcaccgagcaggccatccagctgctcgaaaagatgaaaacatctgccagccctttcttcctggccgtgggctaccacaagcctcacatcccttttcggtaccctaaggaattccaaaaactgtaccccctcgagaacataacactggctcctgaccccgaagtgccagacggcctccctccagtggcttataacccctggatggatatccggcagcgggaagatgtccaggcccttaatatcagcgtgccatacggccctatccctgtggacttccagagaaagatcagacagagctacttcgccagcgtctcctacctggatacacaggtgggaagactgctgtctgccctcgatgatctgcagctggcaaacagcacaatcatcgccttcaccagcgaccacggctgggctctgggcgaacacggcgagtgggccaagtactccaactttgacgtggccacccacgtgccactgatcttctacgtgcccggccggaccgcctctctgccagaggccggagagaaactgtttccatatctggaccccttcgacagcgcctctcagctgatggaacctggcagacaatctatggacctggtggaactggtgtccctgttccctaccctggccggactggctggccttcaagtgcctccacggtgccccgtgccaagcttccacgttgagctgtgcagagagggaaaaaacctgctgaagcacttcagattccgggacctggaggaagatccttacctccctggaaatcctagagagctgatcgcctacagccagtaccccagacccagcgacatccctcagtggaacagcgataagccctccctgaaggacattaagatcatgggatatagcatcagaaccatcgactacagatacaccgtgtgggtcggcttcaaccccgacgagttcctggccaatttcagcgacatccacgccggagaactgtacttcgtggattctgaccctctgcaggaccacaacatgtacaacgacagccagggcggcgatctgttccagctgcttatgccttga

SEQ ID NO: 68 - DNA, 1653 bp, Artificial Sequence
  Synthetic nucleic acid sequence
atgcccccccccagaaccgggagaggcctcctgtggctgggcctggtgctgagctccgtgtgtgtggccctgggcagcgagacccaggccaacagcaccacagacgccctgaatgtgctgctgatcattgtggatgacctgaggcccagcctgggctgctacggcgacaagctggtgcgcagcccaaacattgaccagctggcctcccacagcctgctgtttcagaatgccttcgcccagcaggctgtgtgtgcccccagcagagtgtcctttctgacaggccggcggccagataccaccagactgtatgacttcaattcctattggagagtgcacgctggcaacttcagcaccatcccccagtatttcaaagagaatggctacgtgaccatgtccgtgggcaaggtgttccaccctggcatcagcagcaaccacacagacgatagcccctatagctggtctttcccaccttatcatcccagcagcgagaagtatgaaaacaccaagacctgcaggggccccgacggagaactgcacgccaacctgctgtgccccgtggacgtgctggacgtgcctgagggcaccctgcccgataagcagagcactgagcaggctatccagctgctggagaagatgaagacaagcgcctctcctttcttcctggccgtgggctaccataagcctcacatccccttcaggtaccctaaggagttccagaagctgtatcccctggagaacatcaccctggcccccgacccagaggtgcccgatggcctgccccccgtggcctacaacccatggatggatattcgccagagggaggatgtgcaggctctgaacatcagcgtgccctacggccccatccccgtggacttccagcgcaagatccggcagagctactttgcctccgtgtcttacctggatacacaggtgggcaggctgctgtccgccctggacgatctgcagctggccaatagcaccatcatcgccttcaccagcgaccatggctgggccctgggcgagcacggagaatgggccaagtacagcaactttgacgtggccacccacgtgccacttatcttctacgtgcccggcaggaccgccagcctgcccgaggctggcgagaagctgttcccctacctcgatccttttgattctgcctcccagctgatggagcccggcaggcagagcatggacctggtggagctggtgagcctgtttccaacactggccgggctggccggcctgcaggtgccacccagatgcccagtgcccagttttcatgtggagctgtgtagggagggcaagaatctgctgaagcacttcagattcagggatctggaggaggacccatacctgcctggaaatcccagggagctgatcgcatactcccagtaccccagacctagcgacatccctcagtggaactctgataagcccagcctcaaggacattaagattatgggctacagtatcaggaccatcgactacagatacacagtgtgggtgggatttaacccagacgagttcctggccaatttctccgacatccacgccggcgagctgtacttcgtggactctgaccctctgcaggaccacaacatgtacaatgacagccagggaggagacctgttccagctgctgatgccctga

SEQ ID NO: 69 - DNA, 2118 bp, Artificial Sequence
  Synthetic nucleic acid sequence
aactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaccatgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggtaagggtttaagggatggttggttggtggggtattaatgtttaattacctggagcacctgcctgaaatcactttttttcaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttgactcgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 70 - DNA, 2432 bp, Artificial Sequence
  Synthetic nucleic acid sequence
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtaccaactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaccatgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggtaagggtttaagggatggttggttggtggggtattaatgtttaattacctggagcacctgcctgaaatcactttttttcaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttgactcgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 71 - DNA, 46 bp, Artificial Sequence
  Synthetic nucleic acid sequence
ggaggggtggagtcgtgacgtgaattacgtcatagggttagggagg

SEQ ID NO: 72 - DNA, 191 bp, Artificial Sequence
  Synthetic nucleic acid sequence
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggagg

SEQ ID NO: 73 - DNA, 152 bp, Artificial Sequence
  Synthetic nucleic acid sequence
ctgcgcgctcgctcgctcactgaggccgcccgggcaaagcccgggcgtcgggcgacctttggtcgcccggcctcagtgagcgagcgagcgcgcagagagggagtggggaggggtggagtcgtgacgtgaattacgtcatagggttagggagg

SEQ ID NO: 74 - DNA, 182 bp, Artificial Sequence
  Synthetic nucleic acid sequence
gtagataagtagcatggcgggttaatcattaactacaaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 75 - DNA, 2117 bp, Artificial Sequence
  Synthetic nucleic acid sequence
actcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaccatgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggtaagggtttaagggatggttggttggtggggtattaatgtttaattacctggagcacctgcctgaaatcactttttttcaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttgactcgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 76 - DNA, 2487 bp, Artificial Sequence
  Synthetic nucleic acid sequence
ctgcgcgctcgctcgctcactgaggccgcccgggcaaagcccgggcgtcgggcgacctttggtcgcccggcctcagtgagcgagcgagcgcgcagagagggagtggggaggggtggagtcgtgacgtgaattacgtcatagggttagggaggtcctgcatatgcggccgcaactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaccatgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggtaagggtttaagggatggttggttggtggggtattaatgtttaattacctggagcacctgcctgaaatcactttttttcaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttgactcgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 77 - DNA, 2117 bp, Artificial Sequence; identical to SEQ ID NO: 75
  Synthetic nucleic acid sequence

SEQ ID NO: 78 - DNA, 2526 bp, Artificial Sequence
  Synthetic nucleic acid sequence
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcaactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaccatgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggtaagggtttaagggatggttggttggtggggtattaatgtttaattacctggagcacctgcctgaaatcactttttttcaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttgactcgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

(19) United States
(12) Patent Application Publication
(10) Pub. No.: US 2021/0361778 A1
(43) Pub. Date: Nov. 25, 2021

(54) ADENO-ASSOCIATED VIRUS COMPOSITIONS FOR IDS GENE TRANSFER AND METHODS OF USE THEREOF

(71) Applicant: Homology Medicines, Inc., Bedford, MA (US)

(72) Inventors: Jacinthe GINGRAS, Bedford, MA (US); Kruti PATEL, Bedford, MA (US); Laura Jane SMITH, Bedford, MA (US); Yvonne WHITE, Bedford, MA (US); Serena Nicole DOLLIVE, Bedford, MA (US); Laura VAN LIESHOUT, Bedford, MA (US); Brenda BURNHAM, Bedford, MA (US)

(21) Appl. No.: 17/222,659

(22) Filed: Apr. 5, 2021

Related U.S. Application Data
(60) Provisional application No. 63/145,258, filed on Feb. 3, 2021.
(60) Provisional application No. 63/094,800, filed on Oct. 21, 2020.
(60) Provisional application No. 63/005,833, filed on Apr. 6, 2020.

(51) Int. Cl. A61K 48/00; C12N 15/86; C12N 9/16; A61P 43/00

(52) U.S. Cl. CPC A61K 48/005; C12N 15/86; C12N 9/16; C12Y 301/06013; C12N 2830/00; C12N 2750/14143; C12N 2750/14152; C12N 2830/50; A61P 43/00

(57) ABSTRACT

Provided are adeno-associated virus (AAV) compositions that can restore IDS gene function in cells, and methods for using the these AAV compositions to treat disorders associated with reduction of IDS gene function (e.g., Hunter syndrome). Also provided are compositions, systems and methods for making the AAV compositions.

### RELATED APPLICATIONS

This application claims priority to U.S. Provisional Patent Application Ser. Nos. 63/005,833, filed Apr. 6, 2020, 63/094,800, filed Oct. 21, 2020, and 63/145,258, filed Feb. 3, 2021, the entire disclosures of which are hereby incorporated herein by reference.

### SEQUENCE LISTING

This application contains a sequence listing which has been submitted electronically in ASCII format and is hereby incorporated by reference in its entirety (said ASCII copy, created on Mar. 30, 2021, is named “404217-HMW-037US1 (182916) SL.txt” and is 217,283 bytes in size).

### BACKGROUND

Hunter syndrome, or mucopolysaccharidosis II (MPS II), is a fatal lysosomal storage disorder that results in a severely reduced life expectancy of 10 to 20 years and that has a high unmet medical need. The disease is a rare X-linked genetic disorder that primarily affects males and interferes with the body's ability to break down and recycle specific mucopolysaccharides, also known as glycosaminoglycans (GAGs). Hunter syndrome is caused by gene defects in iduronate-2-sulfatase (IDS), a lysosomal enzyme that is essential for the stepwise degradation of GAGs, heparan sulfates, and dermatan sulfates. IDS defects cause GAGS to build up in cells throughout the body, interfering with proper functioning of certain cells and organs. As the buildup of GAGs continues, signs and symptoms of Hunter syndrome become more visible. These may include: distinct facial features, a large head, an enlarged abdomen, hearing loss, thickening of heart valves leading to a decline in cardiac function, obstructive airway disease, sleep apnea, decreased range of motion and mobility, and enlargement of the liver and spleen. Two-thirds of patients with Hunter syndrome develop central nervous system (CNS) disease resulting in anomalies in neurocognition and behavior. Children as young as 2 to 4 years old can exhibit symptoms such as coarse facial features, skeletal abnormalities, organomegaly (especially of the liver), and cardio-vascular complications with cognitive impairment. The disease incidence of Hunter syndrome in the US is 1:130,000.

Currently, Hunter syndrome can be managed with a few different treatments. Treatments include bone marrow transplants and enzyme replacement therapy (ERT). ERT requires regular administration, such as for Elaprase, which must be administered weekly by infusion lasting between 1-8 hours. Approved ERT treatments are inadequate to treat neurodegeneration associated with two-thirds of Hunter patients. Other ERT treatments are still in clinical testing phase, such as SHP631, a fusion protein of IDS with an antibody that is engineered to cross the blood brain barrier. Other treatments include ex vivo gene therapy, involving the expansion of transduced peripheral blood lymphocytes with the IDS gene, an approach not recommended for patients with cognitive disease. Despite the availability of a few different treatment options, there is no cure for Hunter syndrome.

Gene therapy provides an opportunity to cure Hunter syndrome. Retroviral vectors, including lentiviral vectors, are capable of integrating nucleic acids into host cell genomes, raising safety concerns due to their non-targeted insertion into the genome. For example, there is a risk of the vector disrupting a tumor suppressor gene or activating an oncogene, thereby causing a malignancy. Indeed, in a clinical trial for treating X-linked severe combined immunodeficiency (SCID) by transducing CD34⁺ bone marrow precursors with a gammaretroviral vector, four out often patients developed leukemia (Hacein-Bey-Abina et al., J Clin Invest. (2008) 118(9):3132-42, incorporated by reference herein in its entirety). Non-integrating vectors, on the other hand, often suffer insufficient expression level or inadequate duration of expression in vivo.

Accordingly, there is a need in the art for improved gene therapy compositions and methods that can efficiently and safely restore IDS gene function in patients with Hunter syndrome.

### SUMMARY

Provided herein are adeno-associated virus (AAV) compositions that can restore IDS gene function in cells, and methods for using the same to treat disorders associated with reduction of IDS gene function (e.g., Hunter syndrome). Also provided are compositions, systems and methods for making the AAV compositions.

Accordingly, in one aspect, the instant disclosure provides a recombinant adeno-associated virus (rAAV) comprising: (a) an AAV capsid comprising an AAV capsid protein; and (b) an rAAV genome comprising a transcriptional regulatory element operably linked to an iduronate-2-sulfatase (IDS) intron-inserted coding sequence comprising an intron.

In certain embodiments, the IDS intron-inserted coding sequence encodes a human IDS protein. In certain embodiments, the IDS intron-inserted coding sequence encodes an amino acid sequence set forth in SEQ ID NO: 23.

In certain embodiments, the intron is a heterologous intron. In certain embodiments, the intron has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleotide sequence set forth in SEQ ID NO: 33.

In certain embodiments, the intron is positioned between nucleotides in the IDS intron-inserted coding sequence that correspond to positions 708 and 709 of the IDS coding sequence set forth in SEQ ID NO: 24. In certain embodiments, the IDS intron-inserted coding sequence comprises a nucleotide sequence having at least at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the nucleotide sequence set forth in SEQ ID NO: 25, 59, or 60.

In certain embodiments, the intron is positioned between nucleotides in the IDS intron-inserted coding sequence that correspond to positions 580 and 581 of the IDS coding sequence set forth in SEQ ID NO: 26. In certain embodiments, the IDS intron-inserted coding sequence comprises a nucleotide sequence having at least at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the nucleotide sequence set forth in SEQ ID NO: 27.

In certain embodiments, the IDS intron-inserted coding sequence comprises the nucleotide sequence set forth in SEQ ID NO: 25, 27, 59, or 60.

In certain embodiments, the transcriptional regulatory element comprises one or more of the elements selected from the group consisting of a cytomegalovirus (CMV) enhancer element, cytomegalovirus (CMV) promoter, chicken-β-actin (CBA) promoter, a small chicken-β-actin (SmCBA) promoter, a glyceraldehyde 3-phosphate dehydrogenase (GAPDH) promoter, a beta-glucuronidase (GUSB) promoter, a modified human EF-1α promoter, a CALM1 promoter, a synthetic promoter, and any combination thereof.

In certain embodiments, the transcriptional regulatory element comprises a nucleotide sequence having at least at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a nucleotide sequence set forth in SEQ ID NO: 29, 30, 36, 39, 40, 41, 42, 44, 46, 47, 48, or 55. In certain embodiments, the transcriptional regulatory element comprises the nucleotide sequence set forth in SEQ ID NO: 29.

In certain embodiments, the rAAV genome further comprises a polyadenylation sequence 3′ to the IDS intron-inserted coding sequence. In certain embodiments, the polyadenylation sequence is an exogenous polyadenylation sequence. In certain embodiments, the exogenous polyadenylation sequence is an SV40 polyadenylation sequence. In certain embodiments, the SV40 polyadenylation sequence comprises the nucleotide sequence set forth in SEQ ID NO: 45.

In certain embodiments, the rAAV genome comprises a nucleotide sequence set forth in SEQ ID NO: 37, 43, 52, 54, 61, 63, 65, 69, 75, or 77.

In certain embodiments, the rAAV genome further comprises a 5′ inverted terminal repeat (5′ ITR) nucleotide, and a 3′ inverted terminal repeat (3′ ITR) nucleotide sequence. In certain embodiments, the 5′ ITR nucleotide sequence has at least 95% sequence identity to SEQ ID NO: 18, 20, or 49, and the 3′ ITR nucleotide sequence has at least 95% sequence identity to SEQ ID NO: 14, 19, 21, or 51.

In certain embodiments, the 5′ ITR nucleotide sequence has at least 80% sequence identity to SEQ ID NO: 18, and the 3′ ITR nucleotide sequence has at least 80% sequence identity to SEQ ID NO: 14. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 18, and the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 19. In certain embodiments, the 5′ ITR nucleotide sequence has at least 80% sequence identity to SEQ ID NO: 18, and the 3′ ITR nucleotide sequence has at least 80% sequence identity to SEQ ID NO: 51. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 49, and the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 14. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 49, and the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 19. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 49, and the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 51. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 20, and the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 21. In certain embodiments, the 5′ ITR nucleotide sequence and the 3′ ITR nucleotide, respectively, comprise the sequences of SEQ ID NO: 18 and 14; 18 and 19; 18 and 51; 49 and 14; 49 and 19; 40 and 51; or 20 and 21.

In certain embodiments, the rAAV genome comprises a nucleotide sequence set forth in SEQ ID NO: 28, 38, 50, 53, 56, 57, 58, 62, 64, 66, 70, 71, 72, 73, or 74. In certain embodiments, the rAAV genome comprises the nucleotide sequences set forth in SEQ ID NO: 72 and 74; 72 and 28; 73 and 74; or 73 and 28.

In certain embodiments, the rAAV genome comprises a nucleotide sequence set forth in SEQ ID NO: 38, 50, 62, 64, 66, 70, 76, or 78.

In certain embodiments, the AAV capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 1, 2, 3, 4, 5, 6, 7, 8, 9, 10, 11, 12, 13, 15, 16, or 17. In certain embodiments, the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

In certain embodiments, (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the capsid protein comprises the amino acid sequence of amino acids 203-736 of SEQ ID NO: 1, 2, 3, 4, 5, 6, 7, 8, 9, 10, 11, 12, 13, 15, 16, or 17.

In certain embodiments, the AAV capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 1, 2, 3, 4, 5, 6, 7, 8, 9, 10, 11, 12, 13, 15, 16, or 17. In certain embodiments, the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

In certain embodiments, (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the capsid protein comprises the amino acid sequence of amino acids 138-736 of SEQ ID NO: 1, 2, 3, 4, 5, 6, 7, 8, 9, 10, 11, 12, 13, 15, 16, or 17.

In certain embodiments, the AAV capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 1, 2, 3, 4, 5, 6, 7, 8, 9, 10, 11, 12, 13, 15, 16, or 17. In certain embodiments, the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

In certain embodiments, (a) the amino acid in the capsid protein corresponding to amino acid 2 of SEQ ID NO: 16 is T, and the amino acid in the capsid protein corresponding to amino acid 312 of SEQ ID NO: 16 is Q; (b) the amino acid in the capsid protein corresponding to amino acid 65 of SEQ ID NO: 16 is I, and the amino acid in the capsid protein corresponding to amino acid 626 of SEQ ID NO: 16 is Y; (c) the amino acid in the capsid protein corresponding to amino acid 77 of SEQ ID NO: 16 is R, and the amino acid in the capsid protein corresponding to amino acid 690 of SEQ ID NO: 16 is K; (d) the amino acid in the capsid protein corresponding to amino acid 119 of SEQ ID NO: 16 is L, and the amino acid in the capsid protein corresponding to amino acid 468 of SEQ ID NO: 16 is S; (e) the amino acid in the capsid protein corresponding to amino acid 626 of SEQ ID NO: 16 is G, and the amino acid in the capsid protein corresponding to amino acid 718 of SEQ ID NO: 16 is G; (0 the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (g) the amino acid in the capsid protein corresponding to amino acid 505 of SEQ ID NO: 16 is R, and the amino acid in the capsid protein corresponding to amino acid 687 of SEQ ID NO: 16 is R; (h) the amino acid in the capsid protein corresponding to amino acid 346 of SEQ ID NO: 16 is A, and the amino acid in the capsid protein corresponding to amino acid 505 of SEQ ID NO: 16 is R; or (i) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the capsid protein comprises the amino acid sequence of amino acids 1-736 of SEQ ID NO: 1, 2, 3, 4, 5, 6, 7, 8, 9, 10, 11, 12, 13, 15, 16, or 17.

In another aspect, the instant disclosure provides a method for expressing an iduronate-2-sulfatase (IDS) polypeptide in a cell, the method comprising transducing the cell with a recombinant adeno-associated virus (rAAV) as described herein.

In certain embodiments, the cell is a cell of the central nervous system. In certain embodiments, the cell is a cell of the central nervous system region selected from the group consisting of the spinal cord, the motor cortex, the sensory cortex, the hippocampus, the putamen, the cerebellum optionally the cerebellar nuclei, and any combination thereof. In certain embodiments, the cell is a neuron and/or a glial cell, optionally wherein the cell is a neuron and/or a glial cell of the central nervous system and/or the peripheral nervous system. In certain embodiments, the cell is a cell selected from the group consisting of a motor neuron, an astrocyte, an oligodendrocyte, a cell of the cerebral cortex in the central nervous system, a sensory neuron of the peripheral nervous system, a Schwann cell, and any combination thereof.

In certain embodiments, the cell is a cell of the liver. In certain embodiments, the cell is a cell of the heart. In certain embodiments, the cell is a cell of the lung. In certain embodiments, the cell is a cell of the kidney. In certain embodiments, the cell is a cell of the spleen.

In certain embodiments, the cell is in a mammalian subject and the rAAV is administered to the subject in an amount effective to transduce the cell in the subject.

In another aspect, the instant disclosure provides a pharmaceutical composition comprising an rAAV as described herein.

In another aspect, the instant disclosure provides a method for treating a subject having Hunter Syndrome (HS), the method comprising administering to the subject an effective amount of an rAAV as described herein, or a pharmaceutical composition as described herein.

In certain embodiments, the rAAV or pharmaceutical composition is administered intravenously.

In certain embodiments, Hunter Syndrome (HS) is associated with an iduronate-2-sulfatase (IDS) gene mutation.

In certain embodiments, the subject is a human subject.

In another aspect, the instant disclosure provides a packaging system for preparation of an rAAV, wherein the packaging system comprises: (a) a first nucleotide sequence encoding one or more AAV Rep proteins; (b) a second nucleotide sequence encoding a capsid protein of an rAAV as described herein; and (c) a third nucleotide sequence comprising an rAAV genome sequence of an rAAV as described herein.

In certain embodiments, the packaging system comprises a first vector comprising the first nucleotide sequence and the second nucleotide sequence, and a second vector comprising the third nucleotide sequence.

In certain embodiments, the packaging system further comprises a fourth nucleotide sequence comprising one or more helper virus genes. In certain embodiments, the fourth nucleotide sequence is comprised within a third vector. In certain embodiments, the fourth nucleotide sequence comprises one or more genes from a virus selected from the group consisting of adenovirus, herpes virus, vaccinia virus, and cytomegalovirus (CMV).

In certain embodiments, the first vector, second vector, and/or the third vector is a plasmid.

In another aspect, the instant disclosure provides a method for recombinant preparation of an rAAV, the method comprising introducing the packaging system described herein into a cell under conditions whereby the rAAV is produced.

In another aspect, the instant disclosure provides a polynucleotide comprising a nucleic acid sequence having at least 80%, 81%, 82%, 83%, 84%, 86%, 87%, 88%, 89%, 90%, 91%, 92%, 93%, 94%, 95%, 96%, 97%, 98%, 99% or 100% sequence identity to the nucleotide sequence set forth in SEQ ID NO: 25, 26, 27, 37, 38, 43, 50, 52, 53, 54, 58, 59, 60, 61, 62, 63, 64, 65, 66, 67, 68, 69, 70, 75, 76, 77, or 78. In certain embodiments, the polynucleotide comprises the nucleic acid sequence set forth in SEQ ID NO: 25, 26, 27, 37, 38, 43, 50, 52, 53, 54, 58, 59, 60, 61, 62, 63, 64, 65, 66, 67, 68, 69, 70, 75, 76, 77, or 78. In certain embodiments, the polynucleotide is comprised within a vector, optionally a viral vector (e.g., an AAV vector, a retroviral vector, or an adenoviral vector) or plasmid vector. In another aspect, the instant disclosure provides a recombinant cell comprising the foregoing polynucleotide.

In another aspect, the instant disclosure provides an rAAV as described herein, a pharmaceutical composition as described herein, a polynucleotide as described herein, or a recombinant cell as described herein, for use as a medicament.

In another aspect, the instant disclosure provides an rAAV as described herein, a pharmaceutical composition as described herein, a polynucleotide as described herein, or a recombinant cell as described herein, for use in the treatment of Hunter Syndrome (HS).

In another aspect, the instant disclosure provides an rAAV as described herein, a pharmaceutical composition as described herein, a polynucleotide as described herein, or a recombinant cell as described herein, for use in a method of treating a subject having Hunter Syndrome (HS), the method comprising administering to the subject an effective amount of the rAAV, the pharmaceutical composition, the polynucleotide, or the cell.

### BRIEF DESCRIPTION OF THE DRAWINGS

FIGS. 1A, 1B, 1C, 1D, and 1E are vector maps of the pHM-05205, pHM-05213, pHM-05214, pHM-05216, and pHM-05217 vectors, respectively.

FIGS. 2A and 2B. FIG. 2A is a graph showing the number of vector genomes per ng of DNA of transduced cells in the liver of wild-type and Ids KO hemizygous mice, four weeks post-dosing. FIG. 2B is a graph showing I2S activity expressed as nmol/hr/mg of protein in the liver of wild-type and Ids KO hemizygous mice, four weeks post-dosing. In FIGS. 2A and 2B, WT refers to untreated wild-type mice; MPS II refers to untreated Ids KO hemizygous mice; AAV9-hIDS refers to Ids KO hemizygous mice administered pHM-05205 packaged in AAV9 capsid at a dose of 2e13 vgs/kg; and HSC15-hIDS refers to Ids KO hemizygous mice administered pHM-05205 packaged in AAVHSC15 capsid at a dose of 2e13 vgs/kg. In FIG. 2B, human liver refers to a representative I2S activity level in normal human liver. * indicates statistical significance at p<0.05; *** indicates statistical significance at p<0.001, and **** indicates statistical significance at p<0.0001, as compared to WT. Untreated mice refers to mice administered vehicle.

FIGS. 3A and 3B. FIG. 3A is a graph showing the number of vector genomes per ng of DNA of transduced cells in the brain (fore brain, mid brain, and hind brain) of wild-type and Ids KO hemizygous mice, four weeks post-dosing. FIG. 3B is a graph showing I2S activity expressed as nmol/hr/mg of protein in the forebrain of wild-type and Ids KO hemizygous mice, four weeks post-dosing. In FIGS. 3A and 3B, WT refers to untreated wild-type mice; MPS II refers to untreated Ids KO hemizygous mice; AAV9-hIDS refers to Ids KO hemizygous mice administered pHM-05205 packaged in AAV9 capsid at a dose of 2e13 vgs/kg; and HSC15-hIDS refers to Ids KO hemizygous mice administered pHM-05205 packaged in AAVHSC15 capsid at a dose of 2e13 vgs/kg. In FIG. 3B, human brain refers to a representative I2S activity level in normal adult human brain. n.s indicates not significant. Untreated mice refers to mice administered vehicle.

FIGS. 4A and 4B. FIG. 4A is a graph showing I2S activity levels detected in the liver of Ids KO hemizygous mice administered pHM-05205 packaged in AAV9 capsid (AAV9-hIDS) at a dose of 2e13 vgs/kg, or pHM-05205 packaged in AAVHSC15 capsid (HSC15-hIDS) at a dose of 2e13 vgs/kg, expressed as a percentage of a representative wild-type I2S activity level in mouse liver, four weeks post-dosing. FIG. 4B is a graph showing I2S activity levels detected in the liver of Ids KO hemizygous mice administered pHM-05205 packaged in AAV9 capsid (AAV9-hIDS) at a dose of 2e13 vgs/kg, or pHM-05205 packaged in AAVHSC15 capsid (HSC15-hIDS) at a dose of 2e13 vgs/kg, expressed as a percentage of a representative normal human I2S activity level in liver, four weeks post-dosing. In FIGS. 4A and 4B, * indicates statistical significance at p<0.05.

FIGS. 5A and 5B. FIG. 5A is a graph showing I2S activity levels detected in the brain of Ids KO hemizygous mice administered pHM-05205 packaged in AAV9 capsid (AAV9-hIDS) at a dose of 2e13 vgs/kg, or pHM-05205 packaged in AAVHSC15 capsid (HSC15-hIDS) at a dose of 2e13 vgs/kg, expressed as a percentage of a representative wild-type I2S activity level in mouse brain, four weeks post-dosing. FIG. 5B is a graph showing I2S activity levels detected in the brain of Ids KO hemizygous mice administered pHM-05205 packaged in AAV9 capsid (AAV9-hIDS) at a dose of 2e13 vgs/kg, or pHM-05205 packaged in AAVHSC15 capsid (HSC15-hIDS) at a dose of 2e13 vgs/kg, expressed as a percentage of a representative normal human I2S activity level in brain, four weeks post-dosing.

FIGS. 6A, 6B, and 6C. FIG. 6A is a graph showing GAG levels detected in the liver of wild-type and Ids KO hemizygous mice, four weeks post-dosing. FIG. 6B is a graph showing GAG levels detected in the brain of wild-type and Ids KO hemizygous mice, four weeks post-dosing. FIG. 6C is a graph showing GAG levels detected in the urine of wild-type and Ids KO hemizygous mice, four weeks post-dosing. In FIGS. 6A, 6B, and 6C, WT refers to untreated wild-type mice; MPS II refers to untreated Ids KO hemizygous mice; AAV9-hIDS refers to Ids KO hemizygous mice administered pHM-05205 packaged in AAV9 capsid at a dose of 2e13 vgs/kg; and HSC15-hIDS refers to Ids KO hemizygous mice administered pHM-05205 packaged in AAVHSC15 capsid at a dose of 2e13 vgs/kg. In FIG. 6A, human liver refers to a representative GAG level in human liver. In FIG. 6B, human brain refers to a representative GAG level in human brain. In FIGS. 6A-6C, * indicates statistical significance at p<0.05, and ** indicates statistical significance at p<0.01. Untreated mice refers to mice administered vehicle.

FIGS. 7A and 7B. FIG. 7A is a graph showing expression of hIDS in the liver of wild-type and Ids KO hemizygous mice, normalized to the expression level of mouse GAPDH, four weeks post-dosing. FIG. 7B is a graph showing expression of hIDS in the brain of wild-type and Ids KO hemizygous mice, normalized to the expression level of mouse GAPDH, four weeks post-dosing. In FIGS. 7A and 7B, WT refers to untreated wild-type mice; MPS II refers to untreated Ids KO hemizygous mice; AAV9-hIDS refers to Ids KO hemizygous mice administered pHM-05205 packaged in AAV9 capsid at a dose of 2e13 vgs/kg; and HSC15-hIDS refers to Ids KO hemizygous mice administered pHM-05205 packaged in AAVHSC15 capsid at a dose of 2e13 vgs/kg. In FIG. 7B, human brain refers to a representative IDS expression level in adult normal human brain. Untreated mice refers to mice administered vehicle.

FIGS. 8A, 8B, and 8C. FIG. 8A is a graph showing total GAG levels detected in the urine of wild-type and Ids KO hemizygous mice, over time. FIG. 8B is a graph showing GAG levels detected in the liver of wild-type and Ids KO hemizygous mice, at twelve weeks post-dosing. FIG. 8C is a graph showing I2S activity expressed as nmol/hr/mg of protein in the liver of wild-type and Ids KO hemizygous mice, at twelve weeks post-dosing. In FIGS. 8A, 8B, and 8C, WT refers to untreated wild-type mice; MPS II refers to untreated Ids KO hemizygous mice; and HSC15-hIDS refers to Ids KO hemizygous mice administered pHM-05205 packaged in AAVHSC15 capsid at a dose of 2e13 vgs/kg. In FIGS. 8A-8C, *** indicates statistical significance at p<0.001, and **** indicates statistical significance at p<0.0001. Untreated mice refers to mice administered vehicle.

FIGS. 9A, 9B, and 9C. FIG. 9A is a graph showing GAG levels detected in the brain of wild-type and Ids KO hemizygous mice, at twelve weeks post-dosing. FIG. 9B is a graph showing I2S activity expressed as nmol/hr/mg of protein in the brain of wild-type and Ids KO hemizygous mice, at twelve weeks post-dosing. In FIGS. 9A and 9B, * indicates statistical significance at p<0.05, and ** indicates statistical significance at p<0.01. FIG. 9C is a graph showing I2S activity in the brain of wild-type and Ids KO hemizygous mice at twelve weeks post-dosing expressed as a percentage of representative wild-type mouse I2S activity. In FIGS. 9A, 9B, and 9C, WT refers to untreated wild-type mice; MPS II refers to untreated Ids KO hemizygous mice; and HSC15-hIDS refers to Ids KO hemizygous mice administered pHM-05205 packaged in AAVHSC15 capsid at a dose of 2e13 vgs/kg. Untreated mice refers to mice administered vehicle.

FIGS. 10A, 10B, and 10C are vector maps of the T-004, T-005, and T-006 vectors, respectively.

FIGS. 11A and 11B. FIG. 11A is a graph showing the total GAG levels detected in the urine of wild-type and Ids KO hemizygous mice, at four weeks post-dosing. FIG. 11B is a graph showing the serum I2S activity expressed in nmol/hr/ml detected in wild-type and Ids KO hemizygous mice, at four weeks post-dosing. In FIGS. 11A and 11B, WT refers to untreated wild-type mice; MPS II refers to untreated Ids KO hemizygous mice; AAV9-hIDS refers to Ids KO hemizygous mice administered pHM-05205 packaged in AAV9 capsid at a dose of 2e13 vgs/kg; HSC15-hIDS refers to Ids KO hemizygous mice administered pHM-05205 packaged in AAVHSC15 capsid at a dose of 2e13 vgs/kg; HSC15-T-004 refers to Ids KO hemizygous mice administered T-004 packaged in AAVHSC15 capsid at a dose of 2e13 vgs/kg; HSC15-T-005 refers to Ids KO hemizygous mice administered T-005 packaged in AAVHSC15 capsid at a dose of 2e13 vgs/kg; and HSC15-T-006 refers to Ids KO hemizygous mice administered T-006 packaged in AAVHSC15 capsid at a dose of 2e13 vgs/kg. In FIGS. 11A and 11B, * indicates statistical significance at p<0.05, ** indicates statistical significance at p<0.01, *** indicates statistical significance at p<0.001, and **** indicates statistical significance at p<0.0001. Untreated mice refers to mice administered vehicle.

FIGS. 12A, 12B, 12C, and 12D. FIG. 12A is a graph showing GAG levels detected in the brain of wild-type and Ids KO hemizygous mice, at four weeks post-dosing. FIG. 12B is a graph showing GAG levels detected in the liver of wild-type and Ids KO hemizygous mice, at four weeks post-dosing. FIG. 12C is a graph showing I2S activity detected in the brain of wild-type and Ids KO hemizygous mice, at four weeks post-dosing. FIG. 12D is a graph showing I2S activity detected in the liver of wild-type and Ids KO hemizygous mice, at four weeks post-dosing. In FIGS. 12A, 12B, 12C, and 12D, WT refers to untreated wild-type mice; MPS II refers to untreated Ids KO hemizygous mice; AAV9-hIDS refers to Ids KO hemizygous mice administered pHM-05205 packaged in AAV9 capsid at a dose of 2e13 vgs/kg; HSC15-hIDS refers to Ids KO hemizygous mice administered pHM-05205 packaged in AAVHSC15 capsid at a dose of 2e13 vgs/kg; HSC15-T-004 refers to Ids KO hemizygous mice administered T-004 packaged in AAVHSC15 capsid at a dose of 2e13 vgs/kg; HSC15-T-005 refers to Ids KO hemizygous mice administered T-005 packaged in AAVHSC15 capsid at a dose of 2e13 vgs/kg; and HSC15-T-006 refers to Ids KO hemizygous mice administered T-006 packaged in AAVHSC15 capsid at a dose of 2e13 vgs/kg. In FIGS. 12A, 12B, and 12D, * indicates statistical significance at p<0.05, ** indicates statistical significance at p<0.01, *** indicates statistical significance at p<0.001, and **** indicates statistical significance at p<0.0001. Untreated mice refers to mice administered vehicle.

FIGS. 13A and 13B are graphs showing the body weight of wild-type and Ids KO hemizygous mice up to four weeks post-dosing. In FIGS. 13A and 13B, Group 1: untreated Ids KO hemizygous control; Group 2: Ids KO hemizygous mice administered pHM-05217 packaged in AAVHSC15 capsid at a dose of 2.2e13 vgs/kg; Group 3: Ids KO hemizygous mice administered pHM-05217 packaged in AAVHSC15 capsid at a dose of 6.5e13 vgs/kg; Group 4: Ids KO hemizygous mice administered pHM-05217 packaged in AAVHSC15 capsid at a dose of 1.1e14 vgs/kg; Group 5: wild-type mice control; Group 6: wild-type mice a administered pHM-05217 packaged in AAVHSC15 capsid at a dose of 2.2e13 vgs/kg; and Group 7: wild-type mice a administered pHM-05217 packaged in AAVHSC15 capsid at a dose of 1.1e14 vgs/kg. Untreated mice refers to mice administered vehicle.

FIGS. 14A, 14B, and 14C are graphs showing dose-dependent I2S activity in wild-type mice administered pHM-05217 packaged in AAVHSC15. FIG. 14A is a graph showing serum I2S activity in nmol/hr/ml detected in wild-type and Ids KO hemizygous mice, two weeks post-dosing. FIG. 14B is a graph showing serum I2S activity in nmol/hr/ml detected in wild-type and Ids KO hemizygous mice, four weeks post-dosing. FIG. 14C is a graph showing I2S activity in nmol/hr/mg in the liver of wild-type and Ids KO hemizygous mice, four weeks post-dosing. In FIGS. 14A, 14B, and 14C, WT refers to untreated wild-type mice; MPS II refers to untreated Ids KO hemizygous mice; WT—2.2E+13 refers to wild-type mice administered pHM-05217 packaged in AAVHSC15 at a dose of 2.2e13 vgs/kg; and WT—1.1E+14 refers to wild-type mice administered pHM-05217 packaged in AAVHSC15 at a dose of 1.1e14 vgs/kg. Untreated mice refers to mice administered vehicle.

FIGS. 15A and 15B. FIG. 15A is a graph showing total GAG levels in the brain of wild-type and hemizygous mice, four weeks post-dosing. FIG. 15B is a graph showing total GAG levels in the liver of wild-type and hemizygous mice, four weeks post-dosing. In FIGS. 15A and 15B, WT refers to untreated wild-type mice; MPS II refers to untreated Ids KO hemizygous mice; WT—2.2E+13 refers to wild-type mice administered pHM-05217 packaged in AAVHSC15 at a dose of 2.2e13 vgs/kg; and WT—1.1E+14 refers to wild-type mice administered pHM-05217 packaged in AAVHSC15 at a dose of 1.1e14 vgs/kg. In FIGS. 15A and 15B, *** indicates statistical significance at p<0.001, and **** indicates statistical significance at p<0.0001. Untreated mice refers to mice administered vehicle.

FIGS. 16A and 16B. FIG. 16A is a graph showing the expression level of IDS in the brain of wild-type and Ids KO hemizygous mice, four weeks post-dosing. FIG. 16B is a graph showing the expression level of IDS in the liver of wild-type and Ids KO hemizygous mice, four weeks post-dosing. In FIGS. 16A and 16B, WT refers to untreated wild-type mice; MPS II refers to untreated Ids KO hemizygous mice; MPS II—2.2E+13 refers to Ids KO hemizygous mice administered pHM-05217 packaged in AAVHSC15 at a dose of 2.2e13 vgs/kg; MPS II—6.5E+13 refers to Ids KO hemizygous mice administered pHM-05217 packaged in AAVHSC15 at a dose of 6.5e13 vgs/kg; and MPS II—1.1E+14 refers to Ids KO hemizygous mice administered pHM-05217 packaged in AAVHSC15 at a dose of 1.1e14 vgs/kg. In FIGS. 16A and 16B, * indicates statistical significance at p<0.05, and *** indicates statistical significance at p<0.001. Untreated mice refers to mice administered vehicle.

FIGS. 17A and 17B. FIG. 17A is a graph showing serum I2S activity detected in wild-type and Ids KO hemizygous mice, at two weeks post-dosing. FIG. 17B is a graph showing serum I2S activity detected in wild-type Ids IDS KO hemizygous mice, at four weeks post-dosing. In FIGS. 17A and 17B, WT refers to untreated wild-type mice; MPS II refers to untreated Ids KO hemizygous mice; MPS II—2.2E+13 refers to Ids KO hemizygous mice administered pHM-05217 packaged in AAVHSC15 at a dose of 2.2e13 vgs/kg; MPS II—6.5E+13 refers to Ids KO hemizygous mice administered pHM-05217 packaged in AAVHSC15 at a dose of 6.5e13 vgs/kg; and MPS II—1.1E+14 refers to Ids KO hemizygous mice administered pHM-05217 packaged in AAVHSC15 at a dose of 1.1e14 vgs/kg. In FIGS. 17A and 17B, ** indicates statistical significance at p<0.01, **** indicates statistical significance at p<0.0001, and ns indicates not significant. Untreated mice refers to mice administered vehicle.

FIG. 18 is a graph showing I2S activity detected in the liver of wild-type and Ids KO hemizygous mice, four weeks post-dosing. WT refers to untreated wild-type mice; MPS II refers to untreated Ids KO hemizygous mice; MPS II—2.2E+13 refers to Ids KO hemizygous mice administered pHM-05217 packaged in AAVHSC15 at a dose of 2.2e13 vgs/kg; MPS II—6.5E+13 refers to Ids KO hemizygous mice administered pHM-05217 packaged in AAVHSC15 at a dose of 6.5e13 vgs/kg; and MPS II—1.1E+14 refers to Ids KO hemizygous mice administered pHM-05217 packaged in AAVHSC15 at a dose of 1.1e14 vgs/kg. In FIG. 18, ** indicates statistical significance at p<0.01, and **** indicates statistical significance at p<0.0001. Untreated mice refers to mice administered vehicle.

FIGS. 19A and 19B are graphs showing total GAG levels detected in the urine of wild-type and Ids KO hemizygous mice, normalized to creatinine levels in urine, two weeks (FIG. 19A) and four weeks (FIG. 19B) post-dosing. FIGS. 19C and 19D are graphs showing the levels of GAG heparan sulfate (GAG-HS; “HS”) (FIG. 19C) and GAG dermatan sulfate (GAG-DS; “DS”) (FIG. 19D) in wild-type mice and Ids KO hemizygous mice four weeks post-dosing. WT refers to untreated wild-type mice; MPS II refers to untreated Ids KO hemizygous mice; MPS II—2.2E+13 refers to Ids KO hemizygous mice administered pHM-05217 packaged in AAVHSC15 at a dose of 2.2e13 vgs/kg; MPS II—6.5E+13 refers to Ids KO hemizygous mice administered pHM-05217 packaged in AAVHSC15 at a dose of 6.5e13 vgs/kg; and MPS II—1.1E+14 refers to Ids KO hemizygous mice administered pHM-05217 packaged in AAVHSC15 at a dose of 1.1e14 vgs/kg. In FIGS. 19A-19D, ns indicates no statistical significance, ** indicates statistical significance at p<0.01, *** indicates statistical significance at p<0.001, and **** indicates statistical significance at p<0.0001. Untreated mice refers to mice administered vehicle.

FIGS. 20A, 20B, 20C, 20D, 20E, and 20F are graphs showing the total GAG levels detected in the liver (FIG. 20A), the heart (FIG. 20B), the lung (FIG. 20C), the brain (FIG. 20D), the kidney (FIG. 20E), and the spleen (FIG. 20F) of wild-type and Ids KO hemizygous mice, four weeks post-dosing. In FIGS. 20A, 20B, 20C, 20D, 20E, and 20F, WT refers to untreated wild-type mice; MPS II refers to untreated Ids KO hemizygous mice; MPS II—2.2E+13 refers to Ids KO hemizygous mice administered pHM-05217 packaged in AAVHSC15 at a dose of 2.2e13 vgs/kg; MPS II—6.5E+13 refers to Ids KO hemizygous mice administered pHM-05217 packaged in AAVHSC15 at a dose of 6.5e13 vgs/kg; and MPS II—1.1E+14 refers to Ids KO hemizygous mice administered pHM-05217 packaged in AAVHSC15 at a dose of 1.1e14 vgs/kg. In FIGS. 20A-20F, * indicates statistical significance at p<0.05, ** indicates statistical significance at p<0.01, *** indicates statistical significance at p<0.001, and **** indicates statistical significance at p<0.0001. Untreated mice refers to mice administered vehicle.

FIGS. 21A, 21B, 21C, and 21D. FIG. 21A is a graph showing the number of vector genomes per μg of DNA of transduced cells in the brain, heart, kidney, liver, lung, and spleen tissue of MPS II mice administered pHM-05217 packaged in AAVHSC15 at various doses as indicated, four weeks post-dosing. FIG. 21B is a graph showing normalized silently altered hIDS transcripts detected in brain, heart, kidney, liver, lung, and spleen tissue of MPS II mice administered pHM-05217 packaged in AAVHSC15, at the various indicated doses, four weeks post-dosing. FIG. 21C is a graph showing heparan sulfate levels detected in the brain, kidney, heart, liver, lung, and spleen tissue of MPS II mice administered pHM-05217 packaged in AAVHSC15, at the various indicated doses, four weeks post-dosing. FIG. 21D is a graph showing dermatan sulfate levels detected in the kidney, heart, liver, and lung tissue of MPS II mice administered pHM-05217 packaged in AAVHSC15, at the various indicated doses, four weeks post-dosing. In FIGS. 21C and 21D, wild-type mice and MPS II mice administered vehicle were used as controls. In FIGS. 21C and 21D, * indicates statistical significance at p<0.05, ** indicates statistical significance at p<0.01, **** indicates statistical significance at p<0.000, and ns indicates not significant.

FIGS. 22A, 22B, 22C, and 22D are graphs showing brain tissue-specific vector genome levels (FIG. 22A), normalized silently altered hIDS transcripts in brain tissue (FIG. 22B), brain tissue hI2S activity (FIG. 22C), and brain tissue-specific heparan sulfate levels (FIG. 22D) of MPS II mice administered pHM-05217 packaged in AAVHSC15, at the various indicated doses, four weeks post-dosing. Wild-type mice and MPS II mice administered vehicle were used as controls. In FIGS. 22C and 22D, * indicates statistical significance at p≤0.05, ** indicates statistical significance at p≤0.01, *** indicates statistical significance at p<0.001, and ns indicates not significant.

FIGS. 23A, 23B, and 23C are graphs showing the pixel intensity of LAMP1 protein detected by IHC in the cerebellum (FIG. 23A), spinal cord (FIG. 23B), and hippocampus (FIG. 23C) of MPS II mice administered pHM-05217 packaged in AAVHSC15, at the various indicated doses, four weeks post-dosing. Wild-type mice and MPS II mice administered vehicle were used as controls. In FIGS. 23A-23C, * indicates statistical significance at p≤0.05, ** indicates statistical significance at p≤0.01, *** indicates statistical significance at p<0.001, **** indicates statistical significance at p≤0.0001, and ns indicates not significant.

FIG. 24 is a graph showing serum I2S activity measured in MPS II mice administered pHM-05217 packaged in AAVHSC15, at the various indicated doses, four weeks post-dosing. Wild-type mice and MPS II mice administered vehicle were used as controls. In FIG. 24, ** indicates statistical significance at p<0.01, **** indicates statistical significance at p<0.0001, and ns indicates not significant.

FIG. 25 is a graph showing liver tissue I2S activity measured in MPS II mice administered pHM-05217 packaged in AAVHSC15, at the various indicated doses, four weeks post-dosing. Wild-type mice and MPS II mice administered vehicle were used as controls. In FIG. 25, ** indicates statistical significance at p≤0.01, **** indicates statistical significance at p<0.0001.

FIG. 26A is a vector map of the pHM-05205 vector. FIGS. 26B, 26C, and 26D are graphs showing serum I2S activity (FIG. 26B), liver tissue I2S activity (FIG. 26C), and normalized hIDS transcripts in the brain (FIG. 26D) of MPS II mice administered either pHM-05205 (comprising a wild-type hIDS coding sequence) or pHM-05208 (comprising a silently altered hIDS coding sequence) packaged in AAVHSC15 at a dose of 6e13 vgs/kg, four weeks post-dosing. Wild-type mice and MPS II (also referred to as “Hemi”) administered vehicle were used as controls. In FIGS. 26B-26D, **** indicates statistical significance at p<0.0001, and ns indicates not significant.

FIG. 27A is a vector map of the pHM-05211 vector. FIGS. 27B and 27C. FIG. 27B is a graph showing the level of serum I2S activity detected in MPS II mice administered pHM-05205 or pHM-05211 each packaged in AAVHSC15 capsid at a dose of 2e13 vgs/kg. Serum I2S activity was measured at 6 or 8 weeks post-dosing, as indicated. MPS II mice administered vehicle was used as control. FIG. 27C is a graph showing the level of normalized hIDS transcripts in the brain of MPS II mice administered pHM-05205 or pHM-05211, each packaged in AAVHSC15 capsid, at a dose of 2e13 vgs/kg. Mice were sacrificed and brain hIDS transcripts measured at 2 or 8 weeks post-dosing as indicated. In FIGS. 27B and 27C, ns indicates not significant.

FIGS. 28A-28O are graphs showing various data relating to MPS II mice administered pHM-05217 packaged in AAVHSC15 at a dose of 1.8e14 vgs/kg. FIG. 28A is a graph showing the level of serum I2S activity detected using a fluorometric enzyme assay in treated MPS II mice out to 52 weeks post-dosing. Minimum, maximum and median values among individual mice (n=3-5 mice per group) are displayed in the box with error bars denoting standard deviation. FIG. 28B is a graph showing the number of vector genomes per μg of DNA of transduced cells in brain, heart, liver, spleen, kidney and lung tissue of treated MPS II mice 12, 24, 39, and 52 weeks post-dosing. FIG. 28C is a graph showing the number of hIDS transcripts detected in brain, heart, liver, spleen, kidney and lung tissue of treated MPS II mice at 12, 24, 39, and 52 weeks post-dosing. FIG. 28D is a graph showing the level of heparan sulfate detected in brain, heart, liver, spleen, kidney and lung tissue of treated MPS II mice at 52 weeks post-dosing. FIG. 28E and FIG. 28F are graphs showing the pixel intensity of LAMP1 protein detected by IHC in the spinal cord (FIG. 28E) and hippocampus (FIG. 28F) of treated MPS II mice at 52 weeks post-dosing. FIG. 28G is a graph showing the number of vector genomes per μg of DNA of transduced cells in the trigeminal ganglion of treated MPS II at 39 weeks post-dosing. FIG. 28H is a graph showing the level of I2S activity detected in liver tissue of treated MPS II mice at 12, 24, 39, and 52 weeks post-dosing. FIGS. 28I-28L are graphs showing the level of I2S activity detected in brain tissue of treated MPS II mice at 12 (FIG. 28I), 24 (FIG. 28J), 39 (FIG. 28K), and 52 (FIG. 28L) weeks post-dosing. FIG. 28M is a graph showing the levels of GAG-HS detected in the urine of MPS II mice administered 1.8e14 vgs/kg of pHM-05217 packaged in AAVHSC15 out to 52 weeks post-dosing. FIG. 28N is a graph showing the quantitation of Purkinje cell layer cell density in MPS II mice administered 1.8e14 vgs/kg of pHM-05217 packaged in AAVHSC15 at 52 weeks post-dosing. FIG. 28O is a graph showing the zygomatic arch thickness of treated MPS II mice at 52 weeks post-dosing. In each of FIGS. 28B-28D, and 28E-28M, untreated MPS II and wild-type mice were used as controls. In FIGS. 28J-28L, normal adult human brain tissue was used as an additional control. In each case, * indicates statistical significance at p≤0.05, ** indicates statistical significance at p<0.01, *** indicates statistical significance at p≤0.001, and ns indicates not significant. Untreated mice refers to mice administered vehicle.

FIGS. 29A-29E. FIG. 29A is a schematic showing the location of ankle and paw depth and width measurements. FIGS. 29B-29E are graph showing paw width (FIG. 29B), paw depth (FIG. 29C), ankle width (FIG. 29D), and ankle depth (FIG. 29E), measurements in MPS II mice administered pHM-05217 packaged in AAVHSC15 at a dose of 1.8e14 vgs/kg, at 14, 20, 28, 34, 37, 40, 46, and 52 weeks post-dosing. In each case, wild-type mice and MPS II mice administered vehicle were used as controls.

FIGS. 30A-30E. FIGS. 30A, 30D, and 30E are graphs showing the level of I2S activity detected in the serum (FIG. 30A), liver tissue (FIG. 30D), and brain tissue (FIG. 30F) of MPS II mice administered 1.8e14 vgs/kg of pHM-05217 packaged in AAVHSC15 up to 8 weeks post-dosing. MPS II mice administered vehicle were used as controls. In FIG. 30A, * indicates statistical significance at p≤0.05, ** indicates statistical significance at p≤0.01, *** indicates statistical significance at p≤0.001, and ns indicates not significant. FIGS. 30B and 30C are graphs showing the level of vector genomes (FIG. 30B) and silently altered hIDS transcripts (FIG. 30C) detected in brain, heart, liver, and spleen tissue of MPS II mice administered 1.8e14 vgs/kg of pHM-05217 packaged in AAVHSC15, at 8 days, 2 weeks, and 8 weeks post-dosing, as indicated.

FIGS. 31A, 31B, and 31C are graphs showing the levels of GAG-HS detected in brain, heart, liver, and spleen tissue of MPS II mice administered 1.8e14 vgs/kg of pHM-05217 packaged in AAVHSC15, at 8 days (FIG. 31A), 2 weeks (FIG. 31B), and 8 weeks (FIG. 31C) post-dosing, as indicated. FIG. 31D is a graph showing the levels of GAG-HS detected in the urine of MPS II mice administered 1.8e14 vgs/kg of pHM-05217 packaged in AAVHSC15, at the various time points indicated. In each case, wild-type and MPS II mice administered vehicle were used as controls.

FIGS. 32A and 32B are graphs showing the GAG-HS levels detected by HPLC-MS/MS in the cerebrospinal fluid (CSF) (FIG. 32A) or brain tissue (FIG. 32B) of wild type (WT) mice treated with vehicle, MPS II mice treated with vehicle, and MPS II mice treated with pHM-05217 packaged in AAVHSC15 capsid administered intravenously at a dose of 6e13 vgs/kg (MPS II 6E+13), 1e14 vgs/kg (MPS II 1E+14), or 2e14 vgs/kg (MPS II 2E+14), as indicated. FIG. 32C is a graph showing the level of I2S activity detected in brain tissue of wild type (WT) mice treated with vehicle, MPS II mice treated with vehicle, and MPS II mice treated with pHM-05217 packaged in AAVHSC15 capsid administered intravenously at a dose of 6e13 vgs/kg (MPS II 6E+13), 1e14 vgs/kg (MPS II 1E+14), or 2e14 vgs/kg (MPS II 2E+14), as indicated. Normal adult human brain tissue was used as an additional control (“Human WT”). In FIGS. 32A-32C, * indicates statistical significance at p<0.05, ** indicates statistical significance at p<0.01, *** indicates statistical significance at p<0.001, and **** indicates statistical significance at p<0.0001.

FIG. 33 is a graph showing the level of I2S activity detected in cell lysate of IDS KO HeLa cells incubated with serum obtained from an MPS II mouse 8 days after administration of 1.8e14 vgs/kg of pHM-05217 packaged in AAVHSC15, in the presence or absence of mannose 6-phosphate (M6P). In FIG. 33, * indicates statistical significance at p<0.05, and *** indicates statistical significance at p<0.001.

### DETAILED DESCRIPTION

The instant disclosure provides AAV compositions that can restore IDS gene function in cells, and methods for using the same to treat disorders associated with reduction of IDS gene function (e.g., Hunter syndrome). Also provided are compositions, systems and methods for making the AAV compositions.

### I. DEFINITIONS

As used herein, the terms “recombinant adeno-associated virus” or “rAAV” refers to an AAV comprising a genome lacking functional rep and cap genes.

As used herein, the term “IDS gene” refers to the iduronate-2-sulfatase gene. The human IDS gene is identified by National Center for Biotechnology Information (NCBI) Gene ID 3423. An exemplary nucleotide sequence of the complementary coding sequence of an IDS gene is provided as SEQ ID NO: 24. An exemplary amino acid sequence of an IDS polypeptide is provided as SEQ ID NO: 23.

As used herein, the term “rAAV genome” refers to a nucleic acid molecule (e.g., DNA and/or RNA) comprising the genome sequence of an rAAV. The skilled artisan will appreciate that where an rAAV genome comprises a transgene (e.g., an IDS coding sequence operably linked to a transcriptional regulatory element), the rAAV genome can be in the sense or antisense orientation relative to direction of transcription of the transgene.

As used herein, the term “AAV capsid protein” refers to an AAV VP1, VP2, or VP3 capsid protein. The term “Clade F capsid protein” refers to an AAV VP1, VP2, or VP3 capsid protein that has at least 90% identity with the VP1, VP2, or VP3 amino acid sequences set forth, respectively, in amino acids 1-736, 138-736, and 203-736 of SEQ ID NO: 1 herein.

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches, and does not consider the degree of similarity of amino acids to one another. Note that only internal gaps are included in the length, not gaps at the sequence ends.

As used herein, the term “a disease or disorder associated with an IDS gene mutation” refers to any disease or disorder caused by, exacerbated by, or genetically linked with mutation of an IDS gene. In certain embodiments, the disease or disorder associated with an IDS gene mutation is Hunter syndrome or mucopolysaccharidosis II (MPS II).

As used herein, the term “coding sequence” refers to the portion of a complementary DNA (cDNA) that encodes a polypeptide, starting at the start codon and ending at the stop codon. A gene may have one or more coding sequences due to alternative splicing, alternative translation initiation, and variation within the population. A coding sequence may either be wild-type, silently-altered, or intron-inserted. An exemplary wild-type IDS coding sequence is set forth in SEQ ID NO: 24.

As used herein, the term “silently-altered” refers to alteration of a coding sequence or an intron-inserted coding sequence of a gene (e.g., by nucleotide substitution) without changing the amino acid sequence of the polypeptide encoded by the coding sequence or stuffer-inserted coding sequence. Such silent alteration is advantageous in that it may increase the translation efficiency of a coding sequence, and/or prevent recombination with a corresponding sequence of an endogenous gene when a coding sequence is transduced into a cell. An exemplary silently-altered IDS coding sequence as described herein is set forth in SEQ ID NO: 26, 67, or 68.

As used herein, the term “intron-inserted coding sequence” of a gene refers to a nucleotide sequence comprising one or more introns inserted in a coding sequence of the gene. An intron-inserted coding sequence of a gene is also referred to as an intron-inserted coding sequence comprising an intron. In certain embodiments, at least one of the introns is a nonnative or heterologous intron, i.e., having a sequence different from a native intron of the gene. In certain embodiments, all of the introns in the intron-inserted coding sequence are nonnative introns. A nonnative intron can have the sequence of an intron from a different species or the sequence of an intron in a different gene from the same species or from a different species. Alternatively, or additionally, at least a portion of a nonnative intron sequence can be synthetic. A skilled worker will appreciate that nonnative intron sequences can be designed to mediate RNA splicing by introducing any consensus splicing motifs known in the art. Exemplary consensus splicing motifs are provided in Sibley et al., (2016) Nature Reviews Genetics, 17, 407-21, which is incorporated by reference herein in its entirety. Insertion of a nonnative intron may promote the efficiency and robustness of vector packaging, as such sequences may allow for adjustments of the vector to reach an optimal size (e.g., 4.5-4.8 kb). In certain embodiments, at least one of the introns is a native intron of the gene. In certain embodiments, all of the introns in the intron-inserted coding sequence are native introns of the gene. The nonnative or native introns can be inserted at any internucleotide bonds in the coding sequence. In certain embodiments, one or more nonnative or native introns are inserted at internucleotide bonds predicted to promote efficient splicing (see e.g., Zhang (1998) Human Molecular Genetics, 7(5):919-32, the disclosure of which is incorporated by reference herein in its entirety). In certain embodiments, one or more nonnative or native introns are inserted at internucleotide bonds that link two endogenous exons. Accordingly, in certain embodiments, an intron-inserted coding sequence of a gene comprises one or more introns designed for efficient splicing. In certain embodiments, the one or more introns may be inserted into a coding sequence of a gene to enhance expression of the gene (e.g., through intron-mediated enhancement (IME).

As used herein, the terms “heterologous intron” and “nonnative intron” refers to an intron that is not native to a given gene.

In the instant disclosure, nucleotide positions in an IDS gene are specified relative to the first nucleotide of the start codon. The first nucleotide of a start codon is position 1; the nucleotides 5′ to the first nucleotide of the start codon have negative numbers; the nucleotides 3′ to the first nucleotide of the start codon have positive numbers. An exemplary nucleotide 1 of the human IDS gene is nucleotide 170 of the NCBI Reference Sequence: NG_011900.3 (Accession Region: NG_011900, region 5029 . . . 33347, taxon 9606, chromosome X, map Xq28), and an exemplary nucleotide 3 of the human IDS gene is nucleotide 172 of the NCBI Reference Sequence: NG_011900.3. The nucleotide adjacently 5′ to the start codon is nucleotide-1.

As used herein, the term “transcriptional regulatory element” or “TRE” refers to a cis-acting nucleotide sequence, for example, a DNA sequence, that regulates (e.g., controls, increases, or reduces) transcription of an operably linked nucleotide sequence by an RNA polymerase to form an RNA molecule. A TRE relies on one or more trans-acting molecules, such as transcription factors, to regulate transcription. Thus, one TRE may regulate transcription in different ways when it is in contact with different trans-acting molecules, for example, when it is in different types of cells. A TRE may comprise one or more promoter elements and/or enhancer elements. A skilled artisan would appreciate that the promoter and enhancer elements in a gene may be close in location, and the term “promoter” may refer to a sequence comprising a promoter element and an enhancer element. Thus, the term “promoter” does not exclude an enhancer element in the sequence. The promoter and enhancer elements do not need to be derived from the same gene or species, and the sequence of each promoter or enhancer element may be either identical or substantially identical to the corresponding endogenous sequence in the genome.

As used herein, the term “operably linked” is used to describe the connection between a TRE and a coding sequence to be transcribed. Typically, gene expression is placed under the control of a TRE comprising one or more promoter and/or enhancer elements. The coding sequence is “operably linked” to the TRE if the transcription of the coding sequence is controlled or influenced by the TRE. The promoter and enhancer elements of the TRE may be in any orientation and/or distance from the coding sequence, as long as the desired transcriptional activity is obtained. In certain embodiments, the TRE is upstream from the coding sequence.

As used herein, the term “polyadenylation sequence” refers to a DNA sequence that when transcribed into RNA constitutes a polyadenylation signal sequence. The polyadenylation sequence can be native (e.g., from the IDS gene) or exogenous. The exogenous polyadenylation sequence can be a mammalian or a viral polyadenylation sequence (e.g., an SV40 polyadenylation sequence).

As used herein, “exogenous polyadenylation sequence” refers to a polyadenylation sequence not identical or substantially identical to the endogenous polyadenylation sequence of an IDS gene (e.g., human IDS gene). In certain embodiments, an exogenous polyadenylation sequence is a polyadenylation sequence of a non-IDS gene in the same species (e.g., human). In certain embodiments, an exogenous polyadenylation sequence is a polyadenylation sequence of a different species (e.g., a virus).

As used herein, the term “effective amount” in the context of the administration of an AAV to a subject refers to the amount of the AAV that achieves a desired prophylactic or therapeutic effect.

As used herein, the term “about” or “approximately” when referring to a measurable value, such as the expression level of an IDS protein, encompasses variations of ±20% or ±10%, ±5%, ±1%, or ±0.1% of a given value or range, as are appropriate to perform the methods disclosed herein.

### II. ADENO-ASSOCIATED VIRUS COMPOSITIONS

In one aspect, provided herein are novel rAAV compositions useful for expressing an IDS polypeptide in cells with reduced or otherwise defective IDS gene function. In certain embodiments, the AAV disclosed herein comprise: an AAV capsid comprising a capsid protein (e.g., an AAV Clade F capsid protein); and an rAAV genome comprising a transcriptional regulatory element operably linked to an intron-inserted IDS coding sequence (e.g., a silently altered intron-inserted IDS coding sequence), allowing for extrachromosomal expression of IDS in a cell transduced with the AAV.

A capsid protein from any capsid known in the art can be used in the rAAV compositions disclosed herein, including, without limitation, a capsid protein from an AAV1, AAV2, AAV3, AAV4, AAV5, AAV6, AAV7, AAV8, or AAV9 serotype. For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 1, 2, 3, 4, 5, 6, 7, 8, 9, 10, 11, 12, 13, 15, 16, or 17, wherein: the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 203-736 of SEQ ID NO: 1, 2, 3, 4, 5, 6, 7, 8, 9, 10, 11, 12, 13, 15, 16, or 17.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 1, 2, 3, 4, 5, 6, 7, 8, 9, 10, 11, 12, 13, 15, 16, or 17, wherein: the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 138-736 of SEQ ID NO: 1, 2, 3, 4, 5, 6, 7, 8, 9, 10, 11, 12, 13, 15, 16, or 17.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 1, 2, 3, 4, 5, 6, 7, 8, 9, 10, 11, 12, 13, 15, 16, or 17, wherein: the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 2 of SEQ ID NO: 16 is T, and the amino acid in the capsid protein corresponding to amino acid 312 of SEQ ID NO: 16 is Q. In certain embodiments, the amino acid in the capsid protein corresponding to amino acid 65 of SEQ ID NO: 16 is I, and the amino acid in the capsid protein corresponding to amino acid 626 of SEQ ID NO: 16 is Y. In certain embodiments, the amino acid in the capsid protein corresponding to amino acid 77 of SEQ ID NO: 16 is R, and the amino acid in the capsid protein corresponding to amino acid 690 of SEQ ID NO: 16 is K. In certain embodiments, the amino acid in the capsid protein corresponding to amino acid 119 of SEQ ID NO: 16 is L, and the amino acid in the capsid protein corresponding to amino acid 468 of SEQ ID NO: 16 is S. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 1-736 of SEQ ID NO: 1, 2, 3, 4, 5, 6, 7, 8, 9, 10, 11, 12, 13, 15, 16, or 17.

In certain embodiments, the AAV capsid comprises two or more of: (a) a capsid protein comprising the amino acid sequence of amino acids 203-736 of SEQ ID NO: 1, 2, 3, 4, 6, 7, 10, 11, 12, 13, 15, 16, or 17; (b) a capsid protein comprising the amino acid sequence of amino acids 138-736 of SEQ ID NO: 1, 2, 3, 4, 5, 6, 7, 9, 10, 11, 12, 13, 15, 16, or 17; and (c) a capsid protein comprising the amino acid sequence of amino acids 1-736 of SEQ ID NO: 1, 2, 3, 4, 5, 6, 7, 8, 9, 10, 11, 12, 13, 15, 16, or 17. In certain embodiments, the AAV capsid comprises: (a) a capsid protein having an amino acid sequence consisting of amino acids 203-736 of SEQ ID NO: 1, 2, 3, 4, 6, 7, 10, 11, 12, 13, 15, 16, or 17; (b) a capsid protein having an amino acid sequence consisting of amino acids 138-736 of SEQ ID NO: 1, 2, 3, 4, 5, 6, 7, 9, 10, 11, 12, 13, 15, 16, or 17; and (c) a capsid protein having an amino acid sequence consisting of amino acids 1-736 of SEQ ID NO: 1, 2, 3, 4, 5, 6, 7, 8, 9, 10, 11, 12, 13, 15, 16, or 17.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 8; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 8; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 8. In certain embodiments, the AAV capsid comprises one or more of: (a) a capsid protein comprising the amino acid sequence of amino acids 203-736 of SEQ ID NO: 8; (b) a capsid protein comprising the amino acid sequence of amino acids 138-736 of SEQ ID NO: 8; and (c) a capsid protein comprising the amino acid sequence of amino acids 1-736 of SEQ ID NO: 8. In certain embodiments, the AAV capsid comprises two or more of: (a) a capsid protein comprising the amino acid sequence of amino acids 203-736 of SEQ ID NO: 8; (b) a capsid protein comprising the amino acid sequence of amino acids 138-736 of SEQ ID NO: 8; and (c) a capsid protein comprising the amino acid sequence of amino acids 1-736 of SEQ ID NO: 8. In certain embodiments, the AAV capsid comprises: (a) a capsid protein having an amino acid sequence consisting of amino acids 203-736 of SEQ ID NO: 8; (b) a capsid protein having an amino acid sequence consisting of amino acids 138-736 of SEQ ID NO: 8; and (c) a capsid protein having an amino acid sequence consisting of amino acids 1-736 of SEQ ID NO: 8.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 11; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 11; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 11. In certain embodiments, the AAV capsid comprises one or more of: (a) a capsid protein comprising the amino acid sequence of amino acids 203-736 of SEQ ID NO: 11; (b) a capsid protein comprising the amino acid sequence of amino acids 138-736 of SEQ ID NO: 11; and (c) a capsid protein comprising the amino acid sequence of amino acids 1-736 of SEQ ID NO: 11. In certain embodiments, the AAV capsid comprises two or more of: (a) a capsid protein comprising the amino acid sequence of amino acids 203-736 of SEQ ID NO: 11; (b) a capsid protein comprising the amino acid sequence of amino acids 138-736 of SEQ ID NO: 11; and (c) a capsid protein comprising the amino acid sequence of amino acids 1-736 of SEQ ID NO: 11. In certain embodiments, the AAV capsid comprises: (a) a capsid protein having an amino acid sequence consisting of amino acids 203-736 of SEQ ID NO: 11; (b) a capsid protein having an amino acid sequence consisting of amino acids 138-736 of SEQ ID NO: 11; and (c) a capsid protein having an amino acid sequence consisting of amino acids 1-736 of SEQ ID NO: 11.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 13; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 13; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 13. In certain embodiments, the AAV capsid comprises one or more of: (a) a capsid protein comprising the amino acid sequence of amino acids 203-736 of SEQ ID NO: 13; (b) a capsid protein comprising the amino acid sequence of amino acids 138-736 of SEQ ID NO: 13; and (c) a capsid protein comprising the amino acid sequence of amino acids 1-736 of SEQ ID NO: 13. In certain embodiments, the AAV capsid comprises two or more of: (a) a capsid protein comprising the amino acid sequence of amino acids 203-736 of SEQ ID NO: 13; (b) a capsid protein comprising the amino acid sequence of amino acids 138-736 of SEQ ID NO: 13; and (c) a capsid protein comprising the amino acid sequence of amino acids 1-736 of SEQ ID NO: 13. In certain embodiments, the AAV capsid comprises: (a) a capsid protein having an amino acid sequence consisting of amino acids 203-736 of SEQ ID NO: 13; (b) a capsid protein having an amino acid sequence consisting of amino acids 138-736 of SEQ ID NO: 13; and (c) a capsid protein having an amino acid sequence consisting of amino acids 1-736 of SEQ ID NO: 13.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 203-736 of SEQ ID NO: 16; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 138-736 of SEQ ID NO: 16; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 1-736 of SEQ ID NO: 16. In certain embodiments, the AAV capsid comprises one or more of: (a) a capsid protein comprising the amino acid sequence of amino acids 203-736 of SEQ ID NO: 16; (b) a capsid protein comprising the amino acid sequence of amino acids 138-736 of SEQ ID NO: 16; and (c) a capsid protein comprising the amino acid sequence of amino acids 1-736 of SEQ ID NO: 16. In certain embodiments, the AAV capsid comprises two or more of: (a) a capsid protein comprising the amino acid sequence of amino acids 203-736 of SEQ ID NO: 16; (b) a capsid protein comprising the amino acid sequence of amino acids 138-736 of SEQ ID NO: 16; and (c) a capsid protein comprising the amino acid sequence of amino acids 1-736 of SEQ ID NO: 16. In certain embodiments, the AAV capsid comprises: (a) a capsid protein having an amino acid sequence consisting of amino acids 203-736 of SEQ ID NO: 16; (b) a capsid protein having an amino acid sequence consisting of amino acids 138-736 of SEQ ID NO: 16; and (c) a capsid protein having an amino acid sequence consisting of amino acids 1-736 of SEQ ID NO: 16.

rAAV genomes useful in the AAV compositions disclosed herein generally comprise a transcriptional regulatory element (TRE) operably linked to an intron-inserted IDS coding sequence. In certain embodiments, the rAAV genome comprises a 5′ inverted terminal repeat (5′ ITR) nucleotide sequence 5′ of the TRE and intron-inserted IDS coding sequence, and a 3′ inverted terminal repeat (3′ ITR) nucleotide sequence 3′ of the TRE and intron-inserted IDS coding sequence.

In certain embodiments, the intron-inserted IDS coding sequence comprises all or substantially all of a coding sequence of an IDS gene. In certain embodiments, the rAAV genome comprises a nucleotide sequence encoding SEQ ID NO: 23 and can optionally further comprise an exogenous polyadenylation sequence 3′ to the intron-inserted IDS coding sequence. In certain embodiments, the nucleotide sequence of the intron-inserted IDS coding sequence encoding SEQ ID NO: 23 is wild-type (e.g., having the sequence set forth in SEQ ID NO: 25). In certain embodiments, the nucleotide sequence of the intron-inserted IDS coding sequence encoding SEQ ID NO: 23 is silently-altered (e.g., having the sequence set forth in SEQ ID NO: 27, 59, or 60).

In certain embodiments, the intron-inserted IDS coding sequence encodes a polypeptide comprising all or substantially all of the amino acids sequence of an IDS protein. In certain embodiments, the intron-inserted IDS coding sequence encodes the amino acid sequence of a wild-type IDS protein (e.g., human IDS protein). In certain embodiments, the intron-inserted IDS coding sequence encodes the amino acid sequence of a mutant IDS protein (e.g., human IDS protein), wherein the mutant IDS polypeptide is a functional equivalent of the wild-type IDS polypeptide, i.e., can function as a wild-type IDS polypeptide. In certain embodiments, the functionally equivalent IDS polypeptide further comprises at least one characteristic not found in the wild-type IDS polypeptide, e.g., the ability to resist protein degradation.

In certain embodiments, rAAV genomes useful in the AAV compositions disclosed herein generally comprise a transcriptional regulatory element (TRE) operably linked to an intron-inserted coding sequence encoding for IDS.

The rAAV genome can be used to express IDS in any mammalian cells (e.g., human cells). Thus, the TRE can be active in any mammalian cells (e.g., human cells). In certain embodiments, the TRE is active in a broad range of human cells. Such TREs may comprise constitutive promoter and/or enhancer elements including cytomegalovirus (CMV) promoter/enhancer (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 29, 40, or 46), SV40 promoter, chicken ACTB promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 47), JeT promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 30), smCBA promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 55), human elongation factor 1 alpha (EF1α) promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 39), minute virus of mouse (MVM) intron which comprises transcription factor binding sites (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 33), human phosphoglycerate kinase (PGK1) promoter, human ubiquitin C (Ubc) promoter, human beta actin promoter, human neuron-specific enolase (ENO2) promoter, human beta-glucuronidase (GUSB) promoter, a rabbit beta-globin element (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 41), human calmodulin 1 (CALM1) promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 44), and/or human Methyl-CpG Binding Protein 2 (MeCP2) promoter. Any of these TREs can be combined in any order to drive efficient transcription. For example, an rAAV genome may comprise a CMV enhancer, a CBA promoter, and the splice acceptor from exon 3 of the rabbit beta-globin gene, collectively called a CAG promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 42). For example, an rAAV genome may comprise a hybrid of CMV enhancer and CBA promoter followed by a splice donor and splice acceptor, collectively called a CASI promoter region (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 48).

Alternatively, the TRE may be a tissue-specific TRE, i.e., it is active in specific tissue(s) and/or organ(s). A tissue-specific TRE comprises one or more tissue-specific promoter and/or enhancer elements, and optionally one or more constitutive promoter and/or enhancer elements. A skilled artisan would appreciate that tissue-specific promoter and/or enhancer elements can be isolated from genes specifically expressed in the tissue by methods well known in the art.

In certain embodiments, the TRE is brain-specific (e.g., neuron-specific, glial cell-specific, astrocyte-specific, oligodendrocyte-specific, microglia-specific and/or central nervous system-specific). Exemplary brain-specific TREs may comprise one or more elements from, without limitation, human glial fibrillary acidic protein (GFAP) promoter, human synapsin 1 (SYN1) promoter, human synapsin 2 (SYN2) promoter, human metallothionein 3 (MT3) promoter, and/or human proteolipid protein 1 (PLP1) promoter. More brain-specific promoter elements are disclosed in WO 2016/100575A1, the disclosure of which is incorporated by reference herein in its entirety.

In certain embodiments, the rAAV genome comprises two or more TREs, optionally comprising at least one of the TREs disclosed above. A skilled person in the art would appreciate that any of these TREs can be combined in any order, and combinations of a constitutive TRE and a tissue-specific TRE can drive efficient and tissue-specific transcription.

In certain embodiments, the rAAV vector further comprises an intron 5′ to or inserted in the IDS coding sequence. Such introns can increase transgene expression, for example, by reducing transcriptional silencing and enhancing mRNA export from the nucleus to the cytoplasm. In certain embodiments, the rAAV genome comprises from 5′ to 3′: a non-coding exon, an intron, and the IDS coding sequence. In certain embodiments, an intron sequence is inserted in the IDS coding sequence, optionally wherein the intron is inserted at an internucleotide bond that links two native exons. In certain embodiments, the intron is inserted at an internucleotide bond that links native exon 1 and exon 2.

The intron can comprise a native intron sequence of the IDS gene, an intron sequence from a different species or a different gene from the same species (i.e., nonnative or heterologous intron), and/or a synthetic intron sequence. A skilled worker will appreciate that synthetic intron sequences can be designed to mediate RNA splicing by introducing any consensus splicing motifs known in the art (e.g., in Sibley et al., (2016) Nature Reviews Genetics, 17, 407-21, which is incorporated by reference herein in its entirety). Exemplary intron sequences are provided in Lu et al. (2013) Molecular Therapy 21(5): 954-63, and Lu et al. (2017) Hum. Gene Ther. 28(1): 125-34, which are incorporated by reference herein in their entirety. In certain embodiments, the rAAV genome comprises an SV40 element (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 31) or a minute virus of mouse (MVM) intron (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 33). In certain embodiments, the rAAV genome comprises an SV40 element (e.g., comprising the nucleotide sequence set forth in SEQ ID NO: 31) or a minute virus of mouse (MVM) intron (e.g., comprising the nucleotide sequence set forth in SEQ ID NO: 33). In certain embodiments, the rAAV genome comprises a chimeric intron sequence comprising a combination of chicken and rabbit sequences, comprising partially the untranscribed chicken ACTB (cACTB) promoter, all of cACTB exon 1, partially cACTB intron 1, partially rabbit HBB2 (rHBB2) intron 2, and partially rHBB2 exon 3 (e.g., SEQ ID NO: 32). In certain embodiments, the rAAV genome comprises a chimeric intron sequence (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 32). In certain embodiments, the rAAV genome comprises a chimeric intron sequence (e.g., comprising the nucleotide sequence set forth in SEQ ID NO: 32).

In certain embodiments, the rAAV genome comprises a TRE comprising a CMV enhancer, a CBA promoter, and a chimeric intron sequence (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 36). In certain embodiments, the rAAV genome comprises a TRE comprising SEQ ID NO: 36.

In certain embodiments, the rAAV genome comprises a TRE comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 29. In certain embodiments, the rAAV genome comprises a TRE comprising SEQ ID NO: 29.

In certain embodiments, the rAAV genome disclosed herein further comprises a transcription terminator (e.g., a polyadenylation sequence). In certain embodiments, the transcription terminator is 3′ to the intron-inserted IDS coding sequence. The transcription terminator may be any sequence that effectively terminates transcription, and a skilled artisan would appreciate that such sequences can be isolated from any genes that are expressed in the cell in which transcription of the intron-inserted IDS coding sequence is desired. In certain embodiments, the transcription terminator comprises a polyadenylation sequence. In certain embodiments, the polyadenylation sequence is identical or substantially identical to the endogenous polyadenylation sequence of the human IDS gene. In certain embodiments, the polyadenylation sequence is an exogenous polyadenylation sequence. In certain embodiments, the polyadenylation sequence is an SV40 polyadenylation sequence (e.g., comprising the nucleotide sequence set forth in SEQ ID NO: 34, 35, or 45, or a nucleotide sequence complementary thereto). In certain embodiments, the polyadenylation sequence comprises the sequence set forth in SEQ ID NO: 45.

In certain embodiments, the rAAV genome comprises from 5′ to 3′: a TRE, an intron-inserted IDS coding sequence, and a polyadenylation sequence. In certain embodiments, the TRE has at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of SEQ ID NO: 29, 30, 31, 32, 33, 35, 36, 39, 40, 41, 42, 44, 46, 47, 48, and/or 55; the intron-inserted IDS coding sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 25, 27, 59, or 60; and/or the polyadenylation sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of SEQ ID NO: 34, 35, or 45.

In certain embodiments, the TRE comprises the sequence set forth in SEQ ID NO: 29; the intron-inserted IDS coding sequence comprises the sequence set forth in SEQ ID NO: 25; and/or the polyadenylation sequence comprises the sequence set forth in SEQ ID NO: 45.

In certain embodiments, the TRE comprises the sequence set forth in SEQ ID NO: 29; the intron-inserted IDS coding sequence comprises the sequence set forth in SEQ ID NO: 27; and/or the polyadenylation sequence comprises the sequence set forth in SEQ ID NO: 45.

In certain embodiments, the TRE comprises the sequence set forth in SEQ ID NO: 29; the intron-inserted IDS coding sequence comprises the sequence set forth in SEQ ID NO: 59; and/or the polyadenylation sequence comprises the sequence set forth in SEQ ID NO: 45.

In certain embodiments, the TRE comprises the sequence set forth in SEQ ID NO: 29; the intron-inserted IDS coding sequence comprises the sequence set forth in SEQ ID NO: 60; and/or the polyadenylation sequence comprises the sequence set forth in SEQ ID NO: 45.

In certain embodiments, the rAAV genome comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 37, 43, 52, 54, 61, 63, 65, 69, 75, or 77. In certain embodiments, the rAAV genome comprises the nucleotide sequence set forth in SEQ ID NO: 37, 43, 52, 54, 61, 63, 65, 69, 75, or 77. In certain embodiments, the nucleotide sequence of the rAAV genome consists of the nucleotide sequence set forth in SEQ ID NO: 37, 43, 52, 54, 61, 63, 65, 69, 75, or 77. In certain embodiments, the rAAV genome comprises the nucleotide sequence set forth in SEQ ID NO: 37. In certain embodiments, the nucleotide sequence of the rAAV genome consists of the nucleotide sequence set forth in SEQ ID NO: 37. In certain embodiments, the rAAV genome comprises the nucleotide sequence set forth in SEQ ID NO: 43. In certain embodiments, the nucleotide sequence of the rAAV genome consists of the nucleotide sequence set forth in SEQ ID NO: 43. In certain embodiments, the rAAV genome comprises the nucleotide sequence set forth in SEQ ID NO: 52. In certain embodiments, the nucleotide sequence of the rAAV genome consists of the nucleotide sequence set forth in SEQ ID NO: 52. In certain embodiments, the rAAV genome comprises the nucleotide sequence set forth in SEQ ID NO: 54. In certain embodiments, the nucleotide sequence of the rAAV genome consists of the nucleotide sequence set forth in SEQ ID NO: 54. In certain embodiments, the rAAV genome comprises the nucleotide sequence set forth in SEQ ID NO: 61. In certain embodiments, the nucleotide sequence of the rAAV genome consists of the nucleotide sequence set forth in SEQ ID NO: 61. In certain embodiments, the rAAV genome comprises the nucleotide sequence set forth in SEQ ID NO: 63. In certain embodiments, the nucleotide sequence of the rAAV genome consists of the nucleotide sequence set forth in SEQ ID NO: 63. In certain embodiments, the rAAV genome comprises the nucleotide sequence set forth in SEQ ID NO: 65. In certain embodiments, the nucleotide sequence of the rAAV genome consists of the nucleotide sequence set forth in SEQ ID NO: 65. In certain embodiments, the rAAV genome comprises the nucleotide sequence set forth in SEQ ID NO: 69. In certain embodiments, the nucleotide sequence of the rAAV genome consists of the nucleotide sequence set forth in SEQ ID NO: 69. In certain embodiments, the rAAV genome comprises the nucleotide sequence set forth in SEQ ID NO: 75. In certain embodiments, the nucleotide sequence of the rAAV genome consists of the nucleotide sequence set forth in SEQ ID NO: 75. In certain embodiments, the rAAV genome comprises the nucleotide sequence set forth in SEQ ID NO: 77. In certain embodiments, the nucleotide sequence of the rAAV genome consists of the nucleotide sequence set forth in SEQ ID NO: 77.

In certain embodiments, the rAAV genomes disclosed herein further comprise a 5′ inverted terminal repeat (5′ ITR) nucleotide sequence 5′ of the TRE, and a 3′ inverted terminal repeat (3′ ITR) nucleotide sequence 3′ of the intron-inserted IDS coding sequence. ITR sequences from any AAV serotype or variant thereof can be used in the rAAV genomes disclosed herein. The 5′ and 3′ ITR can be from an AAV of the same serotype or from AAVs of different serotypes. Exemplary ITRs for use in the rAAV genomes disclosed herein are set forth in SEQ ID NO: 14, 18-21, 28, 49, 51, 57, and 72-74 herein.

In certain embodiments, the 5′ ITR or 3′ ITR is from AAV2. In certain embodiments, both the 5′ ITR and the 3′ ITR are from AAV2. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 18, or the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 14. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 18, and the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 14. In certain embodiments, the rAAV genome comprises a nucleotide sequence set forth in any one of SEQ ID NO: 37, 43, 52, or 54, a 5′ ITR nucleotide sequence having the sequence of SEQ ID NO: 18, and a 3′ ITR nucleotide sequence having the sequence of SEQ ID NO: 14.

In certain embodiments, the 5′ ITR or 3′ ITR is from AAV2. In certain embodiments, both the 5′ ITR and the 3′ ITR are from AAV2. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 18, or the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 19. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 18, and the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 19. In certain embodiments, the rAAV genome comprises a nucleotide sequence set forth in any one of SEQ ID NO: 37, 43, 52, or 54, a 5′ ITR nucleotide sequence having the sequence of SEQ ID NO: 18, and a 3′ ITR nucleotide sequence having the sequence of SEQ ID NO: 19.

In certain embodiments, the 5′ ITR or 3′ ITR are from AAV5. In certain embodiments, both the 5′ ITR and 3′ ITR are from AAV5. In certain embodiments, the 5′ ITR nucleotide sequence has at 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 20, or the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 21. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 20, and the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 21. In certain embodiments, the rAAV genome comprises a nucleotide sequence set forth in any one of SEQ ID NO: 37, 43, 52, or 54, a 5′ ITR nucleotide sequence having the sequence of SEQ ID NO: 20, and a 3′ ITR nucleotide sequence having the sequence of SEQ ID NO: 21.

In certain embodiments, the 5′ ITR nucleotide sequence and the 3′ ITR nucleotide sequence are substantially complementary to each other (e.g., are complementary to each other except for mismatch at 1, 2, 3, 4, or 5 nucleotide positions in the 5′ or 3′ ITR).

In certain embodiments, the 5′ ITR or the 3′ ITR is modified to reduce or abolish resolution by Rep protein (“non-resolvable ITR”). In certain embodiments, the non-resolvable ITR comprises an insertion, deletion, or substitution in the nucleotide sequence of the terminal resolution site. Such modification allows formation of a self-complementary, double-stranded DNA genome of the AAV after the rAAV genome is replicated in an infected cell. Exemplary non-resolvable ITR sequences are known in the art (see e.g., those provided in U.S. Pat. Nos. 7,790,154 and 9,783,824, the disclosures of which are incorporated by reference herein in their entirety). In certain embodiments, the 5′ ITR comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 49. In certain embodiments, the 5′ ITR consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 49. In certain embodiments, the 5′ ITR consists of the nucleotide sequence set forth in SEQ ID NO: 49. In certain embodiments, the 3′ ITR comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 51. In certain embodiments, the 5′ ITR consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 51. In certain embodiments, the 3′ ITR consists of the nucleotide sequence set forth in SEQ ID NO: 51. In certain embodiments, the 5′ ITR consists of the nucleotide sequence set forth in SEQ ID NO: 49, and the 3′ ITR consists of the nucleotide sequence set forth in SEQ ID NO: 51. In certain embodiments, the 5′ ITR consists of the nucleotide sequence set forth in SEQ ID NO: 49, and the 3′ ITR consists of the nucleotide sequence set forth in SEQ ID NO: 14.

In certain embodiments, the 5′ ITR is flanked by an additional nucleotide sequence derived from a wild-type AAV2 genomic sequence. In certain embodiments, the 5′ ITR is flanked by an additional 46 bp sequence derived from a wild-type AAV2 sequence that is adjacent to a wild-type AAV2 ITR. In certain embodiments, the additional 46 bp sequence is internal to the 5′ ITR. In certain embodiments, the 46 bp sequence consists of the sequence set forth in SEQ ID NO: 71. In certain embodiments, the 5′ ITR comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 71. In certain embodiments, the 5′ ITR comprises the nucleotide sequence set forth in SEQ ID NO: 72 or 73. In certain embodiments, the nucleotide sequence of the 5′ ITR consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 72 or 73. In certain embodiments, the nucleotide sequence of the 5′ ITR consists of the nucleotide sequence set forth in SEQ ID NO: 72 or 73.

In certain embodiments, the 3′ ITR is flanked by an additional nucleotide sequence derived from a wild-type AAV2 genomic sequence. In certain embodiments, the 3′ ITR is flanked by an additional 37 bp sequence derived from a wild-type AAV2 sequence that is adjacent to a wild-type AAV2 ITR. See, e.g., Savy et al., Human Gene Therapy Methods (2017) 28(5): 277-289 (which is hereby incorporated by reference herein in its entirety). In certain embodiments, the additional 37 bp sequence is internal to the 3′ ITR. In certain embodiments, the 37 bp sequence consists of the sequence set forth in SEQ ID NO: 56. In certain embodiments, the 3′ ITR comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 28, 57, or 74. In certain embodiments, the 3′ ITR comprises the nucleotide sequence set forth in SEQ ID NO: 28, 57, or 74. In certain embodiments, the nucleotide sequence of the 3′ ITR consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 28, 57, or 74. In certain embodiments, the nucleotide sequence of the 3′ ITR consists of the nucleotide sequence set forth in SEQ ID NO: 28, 57, or 74.

In certain embodiments, the rAAV genome comprises from 5′ to 3′: a 5′ ITR; an internal element comprising from 5′ to 3′: a TRE, optionally a non-coding exon and an intron, an intron-inserted IDS coding sequence, and a polyadenylation sequence, as disclosed herein; a non-resolvable ITR; a nucleotide sequence complementary to the internal element; and a 3′ ITR. Such rAAV genome can form a self-complementary, double-stranded DNA genome of the AAV after infection and before replication.

In certain embodiments, the rAAV genome comprises from 5′ to 3′: a 5′ ITR, a TRE, an intron-inserted IDS coding sequence, a polyadenylation sequence, and a 3′ ITR. In certain embodiments, the 5′ ITR has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID: 18, 20, 49, or 73; the TRE has at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of SEQ ID NO: 29, 30, 31, 32, 33, 35, 36, 39, 40, 41, 42, 44, 46, 47, 48, and/or 55; the intron-inserted IDS coding sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 25, 27, 59, or 60; the polyadenylation sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of SEQ ID NO: 34, 35, or 45; and/or the 3′ ITR has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID: 14, 19, 21, 28, 51, 57, or 74. In certain embodiments, the 5′ ITR comprises or consists of a nucleotide sequence selected from the group consisting of SEQ ID NO: 18, 20, 49, or 73; the TRE comprises a nucleotide sequence selected from the group consisting of SEQ ID NO: 29, 30, 31, 32, 33, 35, 36, 39, 40, 41, 42, 44, 46, 47, 48, and/or 55; the intron-inserted IDS coding sequence comprises the sequence set forth in SEQ ID NO: 25, 27, 59, or 60; the polyadenylation sequence comprises a nucleotide sequence selected from the group consisting of SEQ ID NO: 34, 35, or 45; and/or the 3′ ITR comprises or consists of a nucleotide sequence selected from the group consisting of SEQ ID NO: 14, 19, 21, 28, 51, 57, or 74.

In certain embodiments, the 5′ ITR comprises or consists of the sequence set forth in SEQ ID NO: 18 or 49; the TRE comprises the sequence set forth in SEQ ID NO: 29; the intron-inserted IDS coding sequence comprises the sequence set forth in SEQ ID NO: 25, 27, 59, or 60; the polyadenylation sequence comprises the sequence set forth in SEQ ID NO: 45; and/or the 3′ ITR comprises or consists of the sequence set forth in SEQ ID NO: 14 or 51.

In certain embodiments, the 5′ ITR comprises or consists of the sequence set forth in SEQ ID NO: 49; the TRE comprises the sequence set forth in SEQ ID NO: 29; the intron-inserted IDS coding sequence comprises the sequence set forth in SEQ ID NO: 25; the polyadenylation sequence comprises the sequence set forth in SEQ ID NO: 45; and/or the 3′ ITR comprises or consists of the sequence set forth in SEQ ID NO: 51.

In certain embodiments, the 5′ ITR comprises or consists of the sequence set forth in SEQ ID NO: 49; the TRE comprises the sequence set forth in SEQ ID NO: 29; the intron-inserted IDS coding sequence comprises the sequence set forth in SEQ ID NO: 25; the polyadenylation sequence comprises the sequence set forth in SEQ ID NO: 45; and/or the 3′ ITR comprises or consists of the sequence set forth in SEQ ID NO: 14.

In certain embodiments, the 5′ ITR comprises or consists of the sequence set forth in SEQ ID NO: 49; the TRE comprises the sequence set forth in SEQ ID NO: 29; the intron-inserted IDS coding sequence comprises the sequence set forth in SEQ ID NO: 27; the polyadenylation sequence comprises the sequence set forth in SEQ ID NO: 45; and/or the 3′ ITR comprises or consists of the sequence set forth in SEQ ID NO: 14.

In certain embodiments, the 5′ ITR comprises or consists of the sequence set forth in SEQ ID NO: 18; the TRE comprises the sequence set forth in SEQ ID NO: 29; the intron-inserted IDS coding sequence comprises the sequence set forth in SEQ ID NO: 25; the polyadenylation sequence comprises the sequence set forth in SEQ ID NO: 45; and/or the 3′ ITR comprises or consists of the sequence set forth in SEQ ID NO: 14.

In certain embodiments, the 5′ ITR comprises or consists of the sequence set forth in SEQ ID NO: 18; the TRE comprises the sequence set forth in SEQ ID NO: 29; the intron-inserted IDS coding sequence comprises the sequence set forth in SEQ ID NO: 27; the polyadenylation sequence comprises the sequence set forth in SEQ ID NO: 45; and/or the 3′ ITR comprises or consists of the sequence set forth in SEQ ID NO: 14.

In certain embodiments, the 5′ ITR comprises or consists of the sequence set forth in SEQ ID NO: 18; the TRE comprises the sequence set forth in SEQ ID NO: 29; the intron-inserted IDS coding sequence comprises the sequence set forth in SEQ ID NO: 27; the polyadenylation sequence comprises the sequence set forth in SEQ ID NO: 45; and/or the 3′ ITR comprises or consists of the sequence set forth in SEQ ID NO: 19.

In certain embodiments, the 5′ ITR comprises or consists of the sequence set forth in SEQ ID NO: 49; the TRE comprises the sequence set forth in SEQ ID NO: 29; the intron-inserted IDS coding sequence comprises the sequence set forth in SEQ ID NO: 59; the polyadenylation sequence comprises the sequence set forth in SEQ ID NO: 45; and/or the 3′ ITR comprises or consists of the sequence set forth in SEQ ID NO: 14.

In certain embodiments, the 5′ ITR comprises or consists of the sequence set forth in SEQ ID NO: 49; the TRE comprises the sequence set forth in SEQ ID NO: 29; the intron-inserted IDS coding sequence comprises the sequence set forth in SEQ ID NO: 60; the polyadenylation sequence comprises the sequence set forth in SEQ ID NO: 45; and/or the 3′ ITR comprises or consists of the sequence set forth in SEQ ID NO: 14.

In certain embodiments, the rAAV genome comprises a sequence at least 90% (e.g., at least 91%, at least 92%, at least 93%, at least 94%, at least 95%, at least 96%, at least 97%, at least 98%, at least 99%, or 100%) identical to SEQ ID NO: 38, 50, 53, 58, 62, 64, 66, 70, 76, or 78. In certain embodiments, the rAAV genome comprises the nucleotide sequence set forth in SEQ ID NO: 38, 50, 53, 58, 62, 64, 66, 70, 76, or 78. In certain embodiments, the nucleotide sequence of the rAAV genome consists of the nucleotide sequence set forth in SEQ ID NO: 38, 50, 53, 58, 62, 64, 66, 70, 76, or 78. In certain embodiments, the rAAV genome comprises the nucleotide sequence set forth in SEQ ID NO: 38. In certain embodiments, the nucleotide sequence of the rAAV genome consists of the nucleotide sequence set forth in SEQ ID NO: 38. In certain embodiments, the rAAV genome comprises the nucleotide sequence set forth in SEQ ID NO: 50. In certain embodiments, the nucleotide sequence of the rAAV genome consists of the nucleotide sequence set forth in SEQ ID NO: 50. In certain embodiments, the rAAV genome comprises the nucleotide sequence set forth in SEQ ID NO: 53. In certain embodiments, the nucleotide sequence of the rAAV genome consists of the nucleotide sequence set forth in SEQ ID NO: 53. In certain embodiments, the rAAV genome comprises the nucleotide sequence set forth in SEQ ID NO: 58. In certain embodiments, the nucleotide sequence of the rAAV genome consists of the nucleotide sequence set forth in SEQ ID NO: 58. In certain embodiments, the rAAV genome comprises the nucleotide sequence set forth in SEQ ID NO: 62. In certain embodiments, the nucleotide sequence of the rAAV genome consists of the nucleotide sequence set forth in SEQ ID NO: 62. In certain embodiments, the rAAV genome comprises the nucleotide sequence set forth in SEQ ID NO: 64. In certain embodiments, the nucleotide sequence of the rAAV genome consists of the nucleotide sequence set forth in SEQ ID NO: 64. In certain embodiments, the rAAV genome comprises the nucleotide sequence set forth in SEQ ID NO: 66. In certain embodiments, the nucleotide sequence of the rAAV genome consists of the nucleotide sequence set forth in SEQ ID NO: 66. In certain embodiments, the rAAV genome comprises the nucleotide sequence set forth in SEQ ID NO: 70. In certain embodiments, the nucleotide sequence of the rAAV genome consists of the nucleotide sequence set forth in SEQ ID NO: 70. In certain embodiments, the rAAV genome comprises the nucleotide sequence set forth in SEQ ID NO: 76. In certain embodiments, the nucleotide sequence of the rAAV genome consists of the nucleotide sequence set forth in SEQ ID NO: 76. In certain embodiments, the rAAV genome comprises the nucleotide sequence set forth in SEQ ID NO: 78. In certain embodiments, the nucleotide sequence of the rAAV genome consists of the nucleotide sequence set forth in SEQ ID NO: 78.

In certain embodiments, the rAAV comprises: (a) an AAV capsid protein comprising the amino acid sequence of amino acids 203-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a wild-type human intron-inserted IDS coding sequence (e.g., an intron-inserted hIDS coding sequence of SEQ ID NO: 25), an SV40 polyadenylation sequence (e.g., the SV40 polyadenylation sequence of SEQ ID NO: 45), and a 3′ ITR element (e.g., the 3′ ITR of SEQ ID NO: 51); (b) an AAV capsid protein comprising the amino acid sequence of amino acids 138-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a wild-type human intron-inserted IDS coding sequence (e.g., an intron-inserted hIDS coding sequence of SEQ ID NO: 25), an SV40 polyadenylation sequence (e.g., the SV40 polyadenylation sequence of SEQ ID NO: 45), and a 3′ ITR element (e.g., the 3′ ITR of SEQ ID NO: 51); and/or (c) an AAV capsid protein comprising the amino acid sequence of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a wild-type human intron-inserted IDS coding sequence (e.g., an intron-inserted hIDS coding sequence of SEQ ID NO: 25), an SV40 polyadenylation sequence (e.g., the SV40 polyadenylation sequence of SEQ ID NO: 45), and a 3′ ITR element (e.g., the 3′ ITR of SEQ ID NO: 51).

In certain embodiments, the rAAV comprises: (a) an AAV capsid protein comprising the amino acid sequence of amino acids 203-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a wild-type human intron-inserted IDS coding sequence (e.g., an intron-inserted hIDS coding sequence of SEQ ID NO: 25), an SV40 polyadenylation sequence (e.g., the SV40 polyadenylation sequence of SEQ ID NO: 45), and a 3′ ITR element (e.g., the 3′ ITR of SEQ ID NO: 14); (b) an AAV capsid protein comprising the amino acid sequence of amino acids 138-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a wild-type human intron-inserted IDS coding sequence (e.g., an intron-inserted hIDS coding sequence of SEQ ID NO: 25), an SV40 polyadenylation sequence (e.g., the SV40 polyadenylation sequence of SEQ ID NO: 45), and a 3′ ITR element (e.g., the 3′ ITR of SEQ ID NO: 14); and/or (c) an AAV capsid protein comprising the amino acid sequence of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a wild-type human intron-inserted IDS coding sequence (e.g., an intron-inserted hIDS coding sequence of SEQ ID NO: 25), an SV40 polyadenylation sequence (e.g., the SV40 polyadenylation sequence of SEQ ID NO: 45), and a 3′ ITR element (e.g., the 3′ ITR of SEQ ID NO: 14).

In certain embodiments, the rAAV comprises: (a) an AAV capsid protein comprising the amino acid sequence of amino acids 203-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 27), an SV40 polyadenylation sequence (e.g., the SV40 polyadenylation sequence of SEQ ID NO: 45), and a 3′ ITR element (e.g., the 3′ ITR of SEQ ID NO: 14); (b) an AAV capsid protein comprising the amino acid sequence of amino acids 138-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 27), an SV40 polyadenylation sequence (e.g., the SV40 polyadenylation sequence of SEQ ID NO: 45), and a 3′ ITR element (e.g., the 3′ ITR of SEQ ID NO: 14); and/or (c) an AAV capsid protein comprising the amino acid sequence of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 27), an SV40 polyadenylation sequence (e.g., the SV40 polyadenylation sequence of SEQ ID NO: 45), and a 3′ ITR element (e.g., the 3′ ITR of SEQ ID NO: 14).

In certain embodiments, the rAAV comprises: (a) an AAV capsid protein comprising the amino acid sequence of amino acids 203-736 of SEQ ID NO: 16, and an rAAV genome comprising 5′ to 3′ following genetic elements: a 5′ ITR element (e.g., the 5′ ITR of SEQ ID NO: 18), a transcriptional regulatory element (e.g., a TRE comprising the sequence of SEQ ID NO: 29), a wild-type human intron-inserted IDS coding sequence (e.g., an intron-inserted hIDS coding sequence of SEQ ID NO: 25), an SV40 polyadenylation sequence (e.g., the SV40 polyadenylation sequence of SEQ ID NO: 45), and a 3′ ITR element (e.g., the 3′ ITR of SEQ ID NO: 14); (b) an AAV capsid protein comprising the amino acid sequence of amino acids 138-736 of SEQ ID NO: 16, and an rAAV genome comprising 5′ to 3′ following genetic elements: a 5′ ITR element (e.g., the 5′ ITR of SEQ ID NO: 18), a transcriptional regulatory element (e.g., a TRE comprising the sequence of SEQ ID NO: 29), a wild-type human intron-inserted IDS coding sequence (e.g., an intron-inserted hIDS coding sequence of SEQ ID NO: 25), an SV40 polyadenylation sequence (e.g., the SV40 polyadenylation sequence of SEQ ID NO: 45), and a 3′ ITR element (e.g., the 3′ ITR of SEQ ID NO: 14); and/or (c) an AAV capsid protein comprising the amino acid sequence of SEQ ID NO: 16, and an rAAV genome comprising 5′ to 3′ following genetic elements: a 5′ ITR element (e.g., the 5′ ITR of SEQ ID NO: 18), a transcriptional regulatory element (e.g., a TRE comprising the sequence of SEQ ID NO: 29), a wild-type human intron-inserted IDS coding sequence (e.g., an intron-inserted hIDS coding sequence of SEQ ID NO: 25), an SV40 polyadenylation sequence (e.g., the SV40 polyadenylation sequence of SEQ ID NO: 45), and a 3′ ITR element (e.g., the 3′ ITR of SEQ ID NO: 14).

In certain embodiments, the rAAV comprises: (a) an AAV capsid protein comprising the amino acid sequence of amino acids 203-736 of SEQ ID NO: 16, and an rAAV genome comprising 5′ to 3′ following genetic elements: a 5′ ITR element (e.g., the 5′ ITR of SEQ ID NO: 18), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 27), an SV40 polyadenylation sequence (e.g., the SV40 polyadenylation sequence of SEQ ID NO: 45), and a 3′ ITR element (e.g., the 3′ ITR of SEQ ID NO: 14); (b) an AAV capsid protein comprising the amino acid sequence of amino acids 138-736 of SEQ ID NO: 16, and an rAAV genome comprising 5′ to 3′ following genetic elements: a 5′ ITR element (e.g., the 5′ ITR of SEQ ID NO: 18), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 27), an SV40 polyadenylation sequence (e.g., the SV40 polyadenylation sequence of SEQ ID NO: 45), and a 3′ ITR element (e.g., the 3′ ITR of SEQ ID NO: 14); and/or (c) an AAV capsid protein comprising the amino acid sequence of SEQ ID NO: 16, and an rAAV genome comprising 5′ to 3′ following genetic elements: a 5′ ITR element (e.g., the 5′ ITR of SEQ ID NO: 18), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 27), an SV40 polyadenylation sequence (e.g., the SV40 polyadenylation sequence of SEQ ID NO: 45), and a 3′ ITR element (e.g., the 3′ ITR of SEQ ID NO: 14).

In certain embodiments, the rAAV comprises: (a) an AAV capsid protein comprising the amino acid sequence of amino acids 203-736 of SEQ ID NO: 16, and an rAAV genome comprising 5′ to 3′ following genetic elements: a 5′ ITR element (e.g., the 5′ ITR of SEQ ID NO: 18), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 27), an SV40 polyadenylation sequence (e.g., the SV40 polyadenylation sequence of SEQ ID NO: 45), and a 3′ ITR element (e.g., the 3′ ITR of SEQ ID NO: 19); (b) an AAV capsid protein comprising the amino acid sequence of amino acids 138-736 of SEQ ID NO: 16, and an rAAV genome comprising 5′ to 3′ following genetic elements: a 5′ ITR element (e.g., the 5′ ITR of SEQ ID NO: 18), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 27), an SV40 polyadenylation sequence (e.g., the SV40 polyadenylation sequence of SEQ ID NO: 45), and a 3′ ITR element (e.g., the 3′ ITR of SEQ ID NO: 19); and/or (c) an AAV capsid protein comprising the amino acid sequence of SEQ ID NO: 16, and an rAAV genome comprising 5′ to 3′ following genetic elements: a 5′ ITR element (e.g., the 5′ ITR of SEQ ID NO: 18), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 27), an SV40 polyadenylation sequence (e.g., the SV40 polyadenylation sequence of SEQ ID NO: 45), and a 3′ ITR element (e.g., the 3′ ITR of SEQ ID NO: 19).

In certain embodiments, the rAAV comprises: (a) an AAV capsid protein comprising the amino acid sequence of amino acids 203-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 59), an SV40 polyadenylation sequence (e.g., the SV40 polyadenylation sequence of SEQ ID NO: 45), and a 3′ ITR element (e.g., the 3′ ITR of SEQ ID NO: 14); (b) an AAV capsid protein comprising the amino acid sequence of amino acids 138-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 59), an SV40 polyadenylation sequence (e.g., the SV40 polyadenylation sequence of SEQ ID NO: 45), and a 3′ ITR element (e.g., the 3′ ITR of SEQ ID NO: 14); and/or (c) an AAV capsid protein comprising the amino acid sequence of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 59), an SV40 polyadenylation sequence (e.g., the SV40 polyadenylation sequence of SEQ ID NO: 45), and a 3′ ITR element (e.g., the 3′ ITR of SEQ ID NO: 14).

In certain embodiments, the rAAV comprises: (a) an AAV capsid protein comprising the amino acid sequence of amino acids 203-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 60), an SV40 polyadenylation sequence (e.g., the SV40 polyadenylation sequence of SEQ ID NO: 45), and a 3′ ITR element (e.g., the 3′ ITR of SEQ ID NO: 14); (b) an AAV capsid protein comprising the amino acid sequence of amino acids 138-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 60), an SV40 polyadenylation sequence (e.g., the SV40 polyadenylation sequence of SEQ ID NO: 45), and a 3′ ITR element (e.g., the 3′ ITR of SEQ ID NO: 14); and/or (c) an AAV capsid protein comprising the amino acid sequence of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 60), an SV40 polyadenylation sequence (e.g., the SV40 polyadenylation sequence of SEQ ID NO: 45), and a 3′ ITR element (e.g., the 3′ ITR of SEQ ID NO: 14).

In certain embodiments, the rAAV comprises: (a) an AAV capsid protein comprising the amino acid sequence of amino acids 203-736 of SEQ ID NO: 16, and an rAAV genome comprising the nucleotide sequence set forth in any one of SEQ ID NO: 25, 27, 29, 37, 38, 43, 50, 52, 53, 54, 58, 60, 61, 62, 63, 64, 65, 66, 69, 70, 75, 76, 77, or 78; (b) an AAV capsid protein comprising the amino acid sequence of amino acids 138-736 of SEQ ID NO: 16, and an rAAV genome comprising the nucleotide sequence set forth in any one of SEQ ID NO: 25, 27, 29, 37, 38, 43, 50, 52, 53, 54, 58, 60, 61, 62, 63, 64, 65, 66, 69, 70, 75, 76, 77, or 78; and/or (c) an AAV capsid protein comprising the amino acid sequence of SEQ ID NO: 16, and an rAAV genome comprising the nucleotide sequence set forth in any one of SEQ ID NO: 25, 27, 29, 37, 38, 43, 50, 52, 53, 54, 58, 60, 61, 62, 63, 64, 65, 66, 69, 70, 75, 76, 77, or 78.

In another aspect, provided herein is a polynucleotide comprising a nucleic acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to the nucleic acid sequence set forth in SEQ ID NO: 25, 26, 27, 37, 38, 43, 50, 52, 53, 54, 58, 59, 60, 61, 62, 63, 64, 65, 66, 67, 68, 69, 70, 75, 76, 77, or 78. In certain embodiments, the polynucleotide comprises the nucleic acid sequence set forth in SEQ ID NO: 25, 26, 27, 37, 38, 43, 50, 52, 53, 54, 58, 59, 60, 61, 62, 63, 64, 65, 66, 67, 68, 69, 70, 75, 76, 77, or 78. In certain embodiments, the nucleic acid sequence of the polynucleotide consists of the nucleic acid sequence set forth in SEQ ID NO: 25, 26, 27, 37, 38, 43, 50, 52, 53, 54, 58, 59, 60, 61, 62, 63, 64, 65, 66, 67, 68, 69, 70, 75, 76, 77, or 78. In certain embodiments, the polynucleotide comprises the nucleic acid sequence set forth in SEQ ID NO: 25, 26, 27, 37, 38, 43, 50, 52, 53, 54, 58, 59, 60, 61, 62, 63, 64, 65, 66, 67, 68, 69, 70, 75, 76, 77, or 78. In certain embodiments, the nucleic acid sequence of the polynucleotide consists of the nucleic acid sequence set forth in SEQ ID NO: 25, 26, 27, 37, 38, 43, 50, 52, 53, 54, 58, 59, 60, 61, 62, 63, 64, 65, 66, 67, 68, 69, 70, 75, 76, 77, or 78.

Also provided herein is a polynucleotide comprising a nucleic acid sequence that is at least 80% (e.g., at least 85%, 90%, 91%, 92%, 93%, 94%, 95%, 96%, 97%, 98%, or 99%) identical to the nucleic acid sequence set forth in SEQ ID NO: 25, 27, 59, or 60. In certain embodiments, the polynucleotide comprises the nucleic acid sequence set forth in SEQ ID NO: 25, 27, 59, or 60. In certain embodiments, the nucleic acid sequence of the polynucleotide consists of the nucleic acid sequence set forth in SEQ ID NO: 25, 27, 59, or 60. In certain embodiments, the polynucleotide comprises the nucleic acid sequence set forth in SEQ ID NO: 25. In certain embodiments, the nucleic acid sequence of the polynucleotide consists of the nucleic acid sequence set forth in SEQ ID NO: 25. In certain embodiments, the polynucleotide comprises the nucleic acid sequence set forth in SEQ ID NO: 27. In certain embodiments, the nucleic acid sequence of the polynucleotide consists of the nucleic acid sequence set forth in SEQ ID NO: 27. In certain embodiments, the polynucleotide comprises the nucleic acid sequence set forth in SEQ ID NO: 59. In certain embodiments, the nucleic acid sequence of the polynucleotide consists of the nucleic acid sequence set forth in SEQ ID NO: 59. In certain embodiments, the polynucleotide comprises the nucleic acid sequence set forth in SEQ ID NO: 60. In certain embodiments, the nucleic acid sequence of the polynucleotide consists of the nucleic acid sequence set forth in SEQ ID NO: 60.

The polynucleotide can comprise DNA, RNA, modified DNA, modified RNA, or a combination thereof. In certain embodiments, the polynucleotide is comprised within a vector, e.g., a viral vector or a plasmid. Also provided herein is a recombinant cell comprising the polynucleotide or vector.

In another aspect, the instant disclosure provides pharmaceutical compositions comprising an AAV as disclosed herein together with a pharmaceutically acceptable excipient, adjuvant, diluent, vehicle or carrier, or a combination thereof. A “pharmaceutically acceptable carrier” includes any material which, when combined with an active ingredient of a composition, allows the ingredient to retain biological activity and without causing disruptive physiological reactions, such as an unintended immune reaction. Pharmaceutically acceptable carriers include water, phosphate buffered saline, emulsions such as oil/water emulsion, and wetting agents. Compositions comprising such carriers are formulated by well-known conventional methods such as those set forth in Remington's Pharmaceutical Sciences, current Ed., Mack Publishing Co., Easton Pa. 18042, USA; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al, 7th ed., Lippincott, Williams, & Wilkins; and Handbook of Pharmaceutical Excipients (2000) A. H. Kibbe et al, 3rd ed. Amer. Pharmaceutical Assoc.

### III. METHODS OF USE

In another aspect, the instant disclosure provides methods for expressing an IDS polypeptide in a cell. The methods generally comprise transducing the cell with a rAAV as disclosed herein. Such methods are highly efficient at restoring IDS expression. Accordingly, in certain embodiments, the methods disclosed herein involve transducing the cell with a rAAV as disclosed herein.

The methods disclosed herein can be applied to any cell harboring a mutation in the IDS gene. The skilled worker will appreciate that cells that require active endogenous IDS (e.g., endogenous I2S activity) are of particular interest. Accordingly, in certain embodiments, the methods are applied to any cell that has lost endogenous I2S activity.

In certain embodiments, the method is applied to a neuron and/or a glial cell. In certain embodiments, of particular interest are neurons and/or glial cells that require active endogenous IDS (e.g., endogenous I2S activity). In certain embodiments, the method is applied to cells of the central nervous system (CNS), and/or cells of the peripheral nervous system (PNS). In certain embodiments, of particular interest are cells of the central nervous system and/or of the peripheral nervous system that require active endogenous IDS (e.g., endogenous I2S activity). In certain embodiments, of particular interest are cells in the forebrain, midbrain, hindbrain, spinal cord, and any combination thereof. In certain embodiments, of particular interest are cells of a central nervous system region selected from the group consisting of the spinal cord, the motor cortex, the sensory cortex, the thalamus, the hippocampus, the putamen, the cerebellum (e.g., the cerebellar nuclei), and any combination thereof. In certain embodiments, of particular interest are cells of the pons and medulla in the brain, ascending fasciculus of the spinal cord, and any combination thereof. In certain embodiments, of particular interest are cells of a central nervous system (CNS) region selected from the group consisting of the spinal cord, the motor cortex, the sensory cortex, the thalamus, the hippocampus, the putamen, the cerebellum (e.g., the cerebellar nuclei), and any combination thereof, that require active endogenous IDS (e.g., endogenous I2S activity). In certain embodiments, of particular interest are motor neurons and astrocytic profiles in the central nervous system (CNS), oligodendrocytes (ascending fibers) in the CNS, cellular populations of the cerebral cortex in the CNS, and sensory neurons of the peripheral nervous system (PNS). In certain embodiments, of particular interest are oligodendrocytes, such as those in the dorsal fasciculus of the spinal cord. In certain embodiments, of particular interest are glial profiles in the central nervous system, including but not limited to, astrocytes, oligodendrocytes, Schwann cells, and any combination thereof. In certain embodiments, of particular interest are motor neurons, astrocytes, oligodendrocytes, cells of the cerebral cortex in the central nervous system, sensory neurons of the peripheral nervous system, glial cells of the peripheral nervous system (e.g., Schwann cells), and any combination thereof.

In certain embodiments, the method is applied to a liver cell (e.g., a hepatocyte). In certain embodiments, of particular interest are liver cells that require active endogenous IDS (e.g., endogenous I2S activity). In certain embodiments, the method is applied to a heart cell (e.g., a cardiomyocyte). In certain embodiments, of particular interest are heart cells that require active endogenous IDS (e.g., endogenous I2S activity). In certain embodiments, the method is applied to a lung cell (e.g., an airway epithelial cell). In certain embodiments, of particular interest are lung cells that require active endogenous IDS (e.g., endogenous I2S activity). In certain embodiments, the method is applied to a kidney cell (e.g., a renal epithelial cell). In certain embodiments, of particular interest are kidney cells that require active endogenous IDS (e.g., endogenous I2S activity). In certain embodiments, the method is applied to a spleen cell (e.g., a splenocyte). In certain embodiments, of particular interest are spleen cells that require active endogenous IDS (e.g., endogenous I2S activity).

The methods disclosed herein can be performed in vitro for research purposes or can be performed ex vivo or in vivo for therapeutic purposes.

In certain embodiments, the cell to be transduced is in a mammalian subject and the AAV is administered to the subject in an amount effective to transduce the cell in the subject. Accordingly, in certain embodiments, the instant disclosure provides a method for treating a subject having a disease or disorder associated with an IDS gene mutation, the method generally comprising administering to the subject an effective amount of a rAAV as disclosed herein. The subject can be a human subject, a non-human primate subject (e.g., a cynomolgus), or a rodent subject (e.g., a mouse) with an IDS mutation. Any disease or disorder associated with an IDS gene mutation can be treated using the methods disclosed herein. Suitable diseases or disorders include, without limitation, Hunter syndrome.

In certain embodiments, the foregoing methods employ an rAAV comprising: (a) an AAV capsid protein comprising the amino acid sequence of amino acids 203-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a wild-type human intron-inserted IDS coding sequence (e.g., an intron-inserted hIDS coding sequence of SEQ ID NO: 25), an SV40 polyadenylation sequence (e.g., the SV40 polyadenylation sequence of SEQ ID NO: 45), and a 3′ ITR element (e.g., the 3′ ITR of SEQ ID NO: 51); (b) an AAV capsid protein comprising the amino acid sequence of amino acids 138-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a wild-type human intron-inserted IDS coding sequence (e.g., an intron-inserted hIDS coding sequence of SEQ ID NO: 25), an SV40 polyadenylation sequence (e.g., the SV40 polyadenylation sequence of SEQ ID NO: 45), and a 3′ ITR element (e.g., the 3′ ITR of SEQ ID NO: 51); and/or (c) an AAV capsid protein comprising the amino acid sequence of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a wild-type human intron-inserted IDS coding sequence (e.g., an intron-inserted hIDS coding sequence of SEQ ID NO: 25), an SV40 polyadenylation sequence (e.g., the SV40 polyadenylation sequence of SEQ ID NO: 45), and a 3′ ITR element (e.g., the 3′ ITR of SEQ ID NO: 51).

In certain embodiments, the foregoing methods employ an rAAV comprising: (a) an AAV capsid protein comprising the amino acid sequence of amino acids 203-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a wild-type human intron-inserted IDS coding sequence (e.g., an intron-inserted hIDS coding sequence of SEQ ID NO: 25), an SV40 polyadenylation sequence (e.g., the SV40 polyadenylation sequence of SEQ ID NO: 45), and a 3′ ITR element (e.g., the 3′ ITR of SEQ ID NO: 14); (b) an AAV capsid protein comprising the amino acid sequence of amino acids 138-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a wild-type human intron-inserted IDS coding sequence (e.g., an intron-inserted hIDS coding sequence of SEQ ID NO: 25), an SV40 polyadenylation sequence (e.g., the SV40 polyadenylation sequence of SEQ ID NO: 45), and a 3′ ITR element (e.g., the 3′ ITR of SEQ ID NO: 14); and/or (c) an AAV capsid protein comprising the amino acid sequence of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a wild-type human intron-inserted IDS coding sequence (e.g., an intron-inserted hIDS coding sequence of SEQ ID NO: 25), an SV40 polyadenylation sequence (e.g., the SV40 polyadenylation sequence of SEQ ID NO: 45), and a 3′ ITR element (e.g., the 3′ ITR of SEQ ID NO: 14).

In certain embodiments, the foregoing methods employ an rAAV comprising: (a) an AAV capsid protein comprising the amino acid sequence of amino acids 203-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 27), an SV40 polyadenylation sequence (e.g., the SV40 polyadenylation sequence of SEQ ID NO: 45), and a 3′ ITR element (e.g., the 3′ ITR of SEQ ID NO: 14); (b) an AAV capsid protein comprising the amino acid sequence of amino acids 138-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 27), an SV40 polyadenylation sequence (e.g., the SV40 polyadenylation sequence of SEQ ID NO: 45), and a 3′ ITR element (e.g., the 3′ ITR of SEQ ID NO: 14); and/or (c) an AAV capsid protein comprising the amino acid sequence of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 27), an SV40 polyadenylation sequence (e.g., the SV40 polyadenylation sequence of SEQ ID NO: 45), and a 3′ ITR element (e.g., the 3′ ITR of SEQ ID NO: 14).

In certain embodiments, the foregoing methods employ an rAAV comprising: (a) an AAV capsid protein comprising the amino acid sequence of amino acids 203-736 of SEQ ID NO: 16, and an rAAV genome comprising 5′ to 3′ following genetic elements: a 5′ ITR element (e.g., the 5′ ITR of SEQ ID NO: 18), a transcriptional regulatory element (e.g., a TRE comprising the sequence of SEQ ID NO: 29), a wild-type human intron-inserted IDS coding sequence (e.g., an intron-inserted hIDS coding sequence of SEQ ID NO: 25), an SV40 polyadenylation sequence (e.g., the SV40 polyadenylation sequence of SEQ ID NO: 45), and a 3′ ITR element (e.g., the 3′ ITR of SEQ ID NO: 14); (b) an AAV capsid protein comprising the amino acid sequence of amino acids 138-736 of SEQ ID NO: 16, and an rAAV genome comprising 5′ to 3′ following genetic elements: a 5′ ITR element (e.g., the 5′ ITR of SEQ ID NO: 18), a transcriptional regulatory element (e.g., a TRE comprising the sequence of SEQ ID NO: 29), a wild-type human intron-inserted IDS coding sequence (e.g., an intron-inserted hIDS coding sequence of SEQ ID NO: 25), an SV40 polyadenylation sequence (e.g., the SV40 polyadenylation sequence of SEQ ID NO: 45), and a 3′ ITR element (e.g., the 3′ ITR of SEQ ID NO: 14); and/or (c) an AAV capsid protein comprising the amino acid sequence of SEQ ID NO: 16, and an rAAV genome comprising 5′ to 3′ following genetic elements: a 5′ ITR element (e.g., the 5′ ITR of SEQ ID NO: 18), a transcriptional regulatory element (e.g., a TRE comprising the sequence of SEQ ID NO: 29), a wild-type human intron-inserted IDS coding sequence (e.g., an intron-inserted hIDS coding sequence of SEQ ID NO: 25), an SV40 polyadenylation sequence (e.g., the SV40 polyadenylation sequence of SEQ ID NO: 45), and a 3′ ITR element (e.g., the 3′ ITR of SEQ ID NO: 14).

In certain embodiments, the foregoing methods employ an rAAV comprising: (a) an AAV capsid protein comprising the amino acid sequence of amino acids 203-736 of SEQ ID NO: 16, and an rAAV genome comprising 5′ to 3′ following genetic elements: a 5′ ITR element (e.g., the 5′ ITR of SEQ ID NO: 18), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 27), an SV40 polyadenylation sequence (e.g., the SV40 polyadenylation sequence of SEQ ID NO: 45), and a 3′ ITR element (e.g., the 3′ ITR of SEQ ID NO: 14); (b) an AAV capsid protein comprising the amino acid sequence of amino acids 138-736 of SEQ ID NO: 16, and an rAAV genome comprising 5′ to 3′ following genetic elements: a 5′ ITR element (e.g., the 5′ ITR of SEQ ID NO: 18), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 27), an SV40 polyadenylation sequence (e.g., the SV40 polyadenylation sequence of SEQ ID NO: 45), and a 3′ ITR element (e.g., the 3′ ITR of SEQ ID NO: 14); and/or (c) an AAV capsid protein comprising the amino acid sequence of SEQ ID NO: 16, and an rAAV genome comprising 5′ to 3′ following genetic elements: a 5′ ITR element (e.g., the 5′ ITR of SEQ ID NO: 18), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 27), an SV40 polyadenylation sequence (e.g., the SV40 polyadenylation sequence of SEQ ID NO: 45), and a 3′ ITR element (e.g., the 3′ ITR of SEQ ID NO: 14).

In certain embodiments, the foregoing methods employ an rAAV comprising: (a) an AAV capsid protein comprising the amino acid sequence of amino acids 203-736 of SEQ ID NO: 16, and an rAAV genome comprising 5′ to 3′ following genetic elements: a 5′ ITR element (e.g., the 5′ ITR of SEQ ID NO: 18), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 27), an SV40 polyadenylation sequence (e.g., the SV40 polyadenylation sequence of SEQ ID NO: 45), and a 3′ ITR element (e.g., the 3′ ITR of SEQ ID NO: 19); (b) an AAV capsid protein comprising the amino acid sequence of amino acids 138-736 of SEQ ID NO: 16, and an rAAV genome comprising 5′ to 3′ following genetic elements: a 5′ ITR element (e.g., the 5′ ITR of SEQ ID NO: 18), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 27), an SV40 polyadenylation sequence (e.g., the SV40 polyadenylation sequence of SEQ ID NO: 45), and a 3′ ITR element (e.g., the 3′ ITR of SEQ ID NO: 19); and/or (c) an AAV capsid protein comprising the amino acid sequence of SEQ ID NO: 16, and an rAAV genome comprising 5′ to 3′ following genetic elements: a 5′ ITR element (e.g., the 5′ ITR of SEQ ID NO: 18), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 27), an SV40 polyadenylation sequence (e.g., the SV40 polyadenylation sequence of SEQ ID NO: 45), and a 3′ ITR element (e.g., the 3′ ITR of SEQ ID NO: 19).

In certain embodiments, the foregoing methods employ an rAAV comprising: (a) an AAV capsid protein comprising the amino acid sequence of amino acids 203-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 59), an SV40 polyadenylation sequence (e.g., the SV40 polyadenylation sequence of SEQ ID NO: 45), and a 3′ ITR element (e.g., the 3′ ITR of SEQ ID NO: 14); (b) an AAV capsid protein comprising the amino acid sequence of amino acids 138-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 59), an SV40 polyadenylation sequence (e.g., the SV40 polyadenylation sequence of SEQ ID NO: 45), and a 3′ ITR element (e.g., the 3′ ITR of SEQ ID NO: 14); and/or (c) an AAV capsid protein comprising the amino acid sequence of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 59), an SV40 polyadenylation sequence (e.g., the SV40 polyadenylation sequence of SEQ ID NO: 45), and a 3′ ITR element (e.g., the 3′ ITR of SEQ ID NO: 14).

In certain embodiments, the foregoing methods employ an rAAV comprising: (a) an AAV capsid protein comprising the amino acid sequence of amino acids 203-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 60), an SV40 polyadenylation sequence (e.g., the SV40 polyadenylation sequence of SEQ ID NO: 45), and a 3′ ITR element (e.g., the 3′ ITR of SEQ ID NO: 14); (b) an AAV capsid protein comprising the amino acid sequence of amino acids 138-736 of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 60), an SV40 polyadenylation sequence (e.g., the SV40 polyadenylation sequence of SEQ ID NO: 45), and a 3′ ITR element (e.g., the 3′ ITR of SEQ ID NO: 14); and/or (c) an AAV capsid protein comprising the amino acid sequence of SEQ ID NO: 16, and an rAAV genome comprising 5′ to 3′ following genetic elements: a 5′ ITR element (e.g., the 5′ ITR of SEQ ID NO: 49), a transcriptional regulatory element (e.g., a TRE comprising the sequence of SEQ ID NO: 29), a silently altered human intron-inserted IDS coding sequence (e.g., an intron-inserted hIDS coding sequence of SEQ ID NO: 60), an SV40 polyadenylation sequence (e.g., the SV40 polyadenylation sequence of SEQ ID NO: 45), and a 3′ ITR element (e.g., the 3′ ITR of SEQ ID NO: 14).

In certain embodiments, the foregoing methods employ an rAAV comprising: (a) an AAV capsid protein comprising the amino acid sequence of amino acids 203-736 of SEQ ID NO: 16, and an rAAV genome comprising the nucleotide sequence set forth in any one of SEQ ID NO: 25, 27, 37, 38, 43, 50, 52, 53, 54, 58, 59, 60, 61, 62, 63, 64, 65, 66, 69, or 70; (b) an AAV capsid protein comprising the amino acid sequence of amino acids 138-736 of SEQ ID NO: 16, and an rAAV genome comprising the nucleotide sequence set forth in any one of SEQ ID NO: 25, 27, 37, 38, 43, 50, 52, 53, 54, 58, 59, 60, 61, 62, 63, 64, 65, 66, 69, or 70; and/or (c) an AAV capsid protein comprising the amino acid sequence of SEQ ID NO: 16, and an rAAV genome comprising the nucleotide sequence set forth in any one of SEQ ID NO: 25, 27, 37, 38, 43, 50, 52, 53, 54, 58, 59, 60, 61, 62, 63, 64, 65, 66, 69, or 70.

The methods disclosed herein are particularly advantageous in that they are capable of expressing an IDS protein in a cell with high efficiency both in vivo and in vitro. In certain embodiments, the expression level of the IDS protein is at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 100%, or any intervening percentage thereof of the expression level of the endogenous IDS protein in a cell of the same type that does not have a mutation in the IDS gene. In certain embodiments, the expression level of the IDS protein is at least 1.1, 1.2, 1.3, 1.4, 1.5, 1.6, 1.7, 1.8, 1.9, 2, 3, 4, 5, 6, 7, 8, 9, or 10-fold higher than the expression level of the endogenous IDS protein in a cell of the same type that does not have a mutation in the IDS gene. Any methods of determining the expression level of the IDS protein can be employed including, without limitation, ELISA, Western blotting, immunostaining, and mass spectrometry.

In certain embodiments, transduction of a cell with an AAV composition disclosed herein can be performed as provided herein or by any method of transduction known to one of ordinary skill in the art. In certain embodiments, the cell may be contacted with the AAV at a multiplicity of infection (MOI) of 50,000; 100,000; 150,000; 200,000; 250,000; 300,000; 350,000; 400,000; 450,000; or 500,000, or at any MOT that provides for optimal transduction of the cell.

An AAV composition disclosed herein can be administered to a subject by any appropriate route including, without limitation, intravenous, intrathecal, intraperitoneal, subcutaneous, intramuscular, intranasal, topical or intradermal routes. In certain embodiments, the composition is formulated for administration via intravenous injection or subcutaneous injection.

### IV. AAV PACKAGING SYSTEMS

In another aspect, the instant disclosure provides packaging systems for recombinant preparation of a recombinant adeno-associated virus (rAAV) disclosed herein. Such packaging systems generally comprise: first nucleotide encoding one or more AAV Rep proteins; a second nucleotide encoding a capsid protein of any of the AAVs as disclosed herein; and a third nucleotide sequence comprising any of the rAAV genomes as disclosed herein, wherein the packaging system is operative in a cell for enclosing the rAAV genome in the capsid to form the AAV.

In certain embodiments, the packaging system comprises a first vector comprising the first nucleotide sequence encoding the one or more AAV Rep proteins and the second nucleotide sequence encoding the AAV capsid protein, and a second vector comprising the third nucleotide sequence comprising the rAAV genome. As used in the context of a packaging system as described herein, a “vector” refers to a nucleic acid molecule that is a vehicle for introducing nucleic acids into a cell (e.g., a plasmid, a virus, a cosmid, an artificial chromosome, etc.).

Any AAV Rep protein can be employed in the packaging systems disclosed herein. In certain embodiments of the packaging system, the Rep nucleotide sequence encodes an AAV2 Rep protein. Suitable AAV2 Rep proteins include, without limitation, Rep 78/68 or Rep 68/52. In certain embodiments of the packaging system, the nucleotide sequence encoding the AAV2 Rep protein comprises a nucleotide sequence that encodes a protein having a minimum percent sequence identity to the AAV2 Rep amino acid sequence of SEQ ID NO: 22, wherein the minimum percent sequence identity is at least 70% (e.g., at least 75%, at least 80%, at least 85%, at least 90%, at least 95%, at least 98%, at least 99%, or 100%) across the length of the amino acid sequence of the AAV2 Rep protein. In certain embodiments of the packaging system, the AAV2 Rep protein has the amino acid sequence set forth in SEQ ID NO: 22.

In certain embodiments of the packaging system, the packaging system further comprises a fourth nucleotide sequence comprising one or more helper virus genes. In certain embodiments of the packaging system, the packaging system further comprises a third vector, e.g., a helper virus vector, comprising the fourth nucleotide sequence comprising the one or more helper virus genes. The third vector may be an independent third vector, integral with the first vector, or integral with the second vector.

In certain embodiments of the packaging system, the helper virus is selected from the group consisting of adenovirus, herpes virus (including herpes simplex virus (HSV)), poxvirus (such as vaccinia virus), cytomegalovirus (CMV), and baculovirus. In certain embodiments of the packaging system, where the helper virus is adenovirus, the adenovirus genome comprises one or more adenovirus RNA genes selected from the group consisting of E1, E2, E4 and VA. In certain embodiments of the packaging system, where the helper virus is HSV, the HSV genome comprises one or more of HSV genes selected from the group consisting of UL5/8/52, ICPO, ICP4, ICP22 and UL30/UL42.

In certain embodiments of the packaging system, the first, second, and/or third vector are contained within one or more plasmids. In certain embodiments, the first vector and the third vector are contained within a first plasmid. In certain embodiments the second vector and the third vector are contained within a second plasmid.

In certain embodiments of the packaging system, the first, second, and/or third vector are contained within one or more recombinant helper viruses. In certain embodiments, the first vector and the third vector are contained within a recombinant helper virus. In certain embodiments, the second vector and the third vector are contained within a recombinant helper virus.

In a further aspect, the disclosure provides a method for recombinant preparation of an AAV as described herein, wherein the method comprises transfecting or transducing a cell with a packaging system as described herein under conditions operative for enclosing the rAAV genome in the capsid to form the rAAV as described herein. Exemplary methods for recombinant preparation of an rAAV include transient transfection (e.g., with one or more transfection plasmids containing a first, and a second, and optionally a third vector as described herein), viral infection (e.g. with one or more recombinant helper viruses, such as a adenovirus, poxvirus (such as vaccinia virus), herpes virus (including HSV, cytomegalovirus, or baculovirus, containing a first, and a second, and optionally a third vector as described herein), and stable producer cell line transfection or infection (e.g., with a stable producer cell, such as a mammalian or insect cell, containing a Rep nucleotide sequence encoding one or more AAV Rep proteins and/or a Cap nucleotide sequence encoding one or more AAV capsid proteins as described herein, and with an rAAV genome as described herein being delivered in the form of a plasmid or a recombinant helper virus).

Accordingly, the instant disclosure provides a packaging system for preparation of a recombinant AAV (rAAV), wherein the packaging system comprises a first nucleotide sequence encoding one or more AAV Rep proteins; a second nucleotide sequence encoding a capsid protein of any one of the AAVs described herein; a third nucleotide sequence comprising an rAAV genome sequence of any one of the AAVs described herein; and optionally a fourth nucleotide sequence comprising one or more helper virus genes.

### V. EXAMPLES

The recombinant AAV vectors disclosed herein mediate highly efficient gene transfer in vitro and in vivo. The following examples demonstrate the efficient restoration of the expression of the IDS gene (which is mutated in certain human diseases, such as Hunter Syndrome) using an AAV-based vector as disclosed herein. These examples are offered by way of illustration, and not by way of limitation.

In examples 5, 6, and 11 below, the 2.2e13 vgs/kg, 6.5e13 vgs/kg, and 1.1e14 vgs/kg doses of AAV are titered with respect to the human IDS gene in the vector genome. When titered using the SV40 polyA sequence in the vector genome, the equivalent doses of AAV are 2e13 vgs/kg, 6e13 vgs/kg, and 1e14 vgs/kg. In examples 9, 10, and 12 below, the 1.8e14 vgs/kg dose of AAV is titered with respect to the human IDS gene in the vector genome. When titred with respect to the SV40 polyA sequence in the vector genome, the equivalent dose of AAV is 1e14 vgs/kg.

### Example 1: Human IDS Transfer Vectors

This example provides human IDS transfer vectors pHM-05205, pHM-05213, pHM-05214, pHM-05216, and pHM-05217 for expression of human IDS (hIDS) in a cell (e.g., a human cell or a mouse cell) into which the vector is transduced.

a) pHM-05205

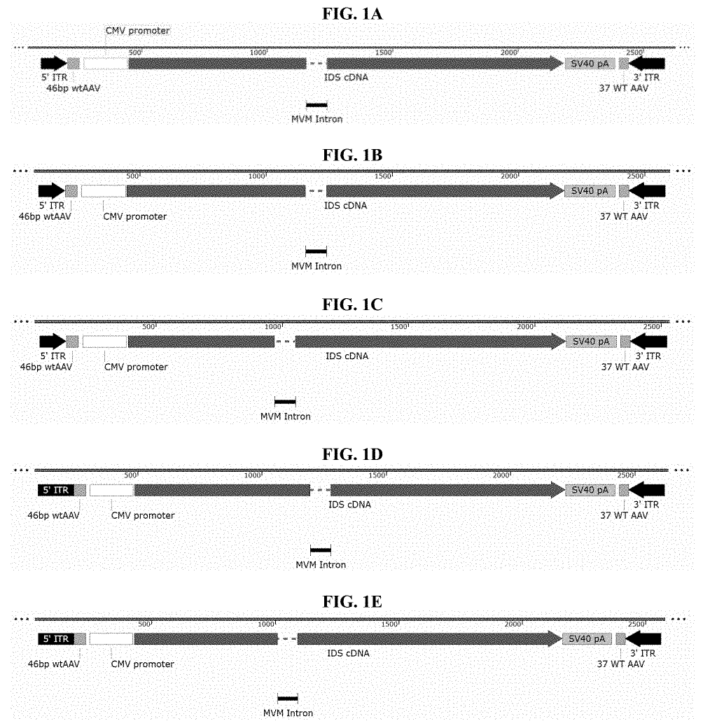

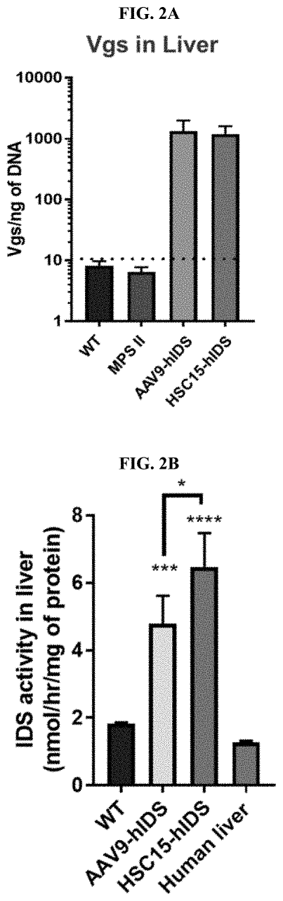

IDS transfer vector pHM-05205, as shown in FIG. 1A, comprises 5′ to 3′ the following genetic elements: a 5′ ITR element; a transcriptional regulatory element comprising a CMV promoter; a wild-type human IDS intron-inserted coding sequence; an SV40 polyadenylation sequence; and a 3′ ITR element. The sequences of these elements are set forth in Table 1. This vector is capable of expressing a human IDS protein in a cell (e.g., a human cell or a mouse cell) into which the vector is transduced.

b) pHM-05213

IDS transfer vector pHM-05213, as shown in FIG. 1B, comprises 5′ to 3′ the following genetic elements: a 5′ ITR element; a transcriptional regulatory element comprising a CMV promoter; a wild-type human IDS intron-inserted coding sequence; an SV40 polyadenylation sequence; and a 3′ ITR element. The sequences of these elements are set forth in Table 1. This vector is capable of expressing a human IDS protein in a cell (e.g., a human cell or a mouse cell) into which the vector is transduced.

c) pHM-05214

IDS transfer vector pHM-05214, as shown in FIG. 1C, comprises 5′ to 3′ the following genetic elements: a 5′ ITR element; a transcriptional regulatory element comprising a CMV promoter; a silently-altered human IDS intron-inserted coding sequence; an SV40 polyadenylation sequence; and a 3′ ITR element. The sequences of these elements are set forth in Table 1. This vector is capable of expressing a human IDS protein in a cell (e.g., a human cell or a mouse cell) into which the vector is transduced.

d) pHM-05216

IDS transfer vector pHM-05216, as shown in FIG. 1D, comprises 5′ to 3′ the following genetic elements: a 5′ ITR element; a transcriptional regulatory element comprising a CMV promoter; a wild-type human IDS intron-inserted coding sequence; an SV40 polyadenylation sequence; and a 3′ ITR element. The sequences of these elements are set forth in Table 1. This vector is capable of expressing a human IDS protein in a cell (e.g., a human cell or a mouse cell) into which the vector is transduced.

e) pHM-05217

IDS transfer vector pHM-05217, as shown in FIG. 1E, comprises 5′ to 3′ the following genetic elements: a 5′ ITR element; a transcriptional regulatory element comprising a CMV promoter; a silently-altered human IDS intron-inserted coding sequence; an SV40 polyadenylation sequence; and a 3′ ITR element. The sequences of these elements are set forth in Table 1. This vector is capable of expressing a human IDS protein in a cell (e.g., a human cell or a mouse cell) into which the vector is transduced.

TABLE 1
Genetic elements in human IDS transfer vectors pHM-05210,
pHM-05213, pHM-05214, pHM-05216, and pHM-05217

| Genetic | pHM-05205 | pHM-05213 | pHM-05214 | pHM-05216 | pHM-05217 |
|---|---|---|---|---|---|
| element | SEQ ID NO: | | | | |
| 5′ ITR element | 49 | 49 | 49 | 18 | 18 |
| Transcriptional | 29 | 29 | 29 | 29 | 29 |
| regulatory element | | | | | |
| Human IDS coding | 25 | 25 | 27 | 25 | 27 |
| sequence | | | | | |
| SV40 | 45 | 45 | 45 | 45 | 45 |
| polyadenylation | | | | | |
| sequence | | | | | |
| 3′ ITR element | 14 | 14 | 14 | 14 | 19 |
| rAAV genome (from | 75 | 37 | 43 | 52 | 54 |
| promoter to polyA | | | | | |
| sequence) | | | | | |
| rAAV genome (from | 76 | 38 | 50 | 53 | 58 |
| 5′ ITR to 3′ ITR) | | | | | |

The vectors disclosed herein can be packaged in an AAV capsid, e.g., an AAV Glade F capsid, such as, without limitation, an AAVHSC5, AAVHSC7, AAVHSC15, or AAVHSC17 capsid. The packaged viral particles can be administered to a wild-type animal, or an IDS-deficient animal.

### Example 2: IDS Gene Transfer in a Mucopolysaccharidosis (MPS) Type II (Hunter Syndrome) Mouse Model

Hunter Syndrome is a rare X-linked genetic disorder, predominately a disease affecting males. The disease is caused by gene defects in the lysosomal enzyme iduronate-2-sulfatase (IDS). IDS is essential for the stepwise degradation of glycosaminoglycans (GAGs), heparan sulfates (HSs), and dermatan sulfates (DSs). IDS is predominately expressed in the central nervous system.

In order to study the effect of IDS gene transfer in vivo, an MPS II mouse model was used. The MPS II mouse model B6J.Cg-Ids^{tm1Muen}/HMI is an Ids knock-out (Ids KO) mouse comprising a deletion in exon 4 and part of exon 5 of the murine Ids gene, abolishing gene expression. See, Garcia et al. (2007) J. Inherit. Metab. Dis. 30(6): 924-934. Ids KO mice lack I2S activity and exhibit increased tissue and organ GAG levels, as well as urine GAG excretion. LAMP1 expression is elevated in the tissues of Ids KO mice. Ids KO mice exhibit progressive skeletal abnormalities, such as thickened digits, and swollen hocks.

In this example, wild-type and Ids KO hemizygous (Ids KO hemi) males, 7-9 weeks of age, were used. A single dose of 2e13 vector genomes per kilogram body weight (vgs/kg) of pHM-05205 packaged in AAVHSC15 capsid or pHM-05205 packaged in AAV9 capsid was administered intravenously to the mice. Mice were sacrificed 4 weeks post-dosing.

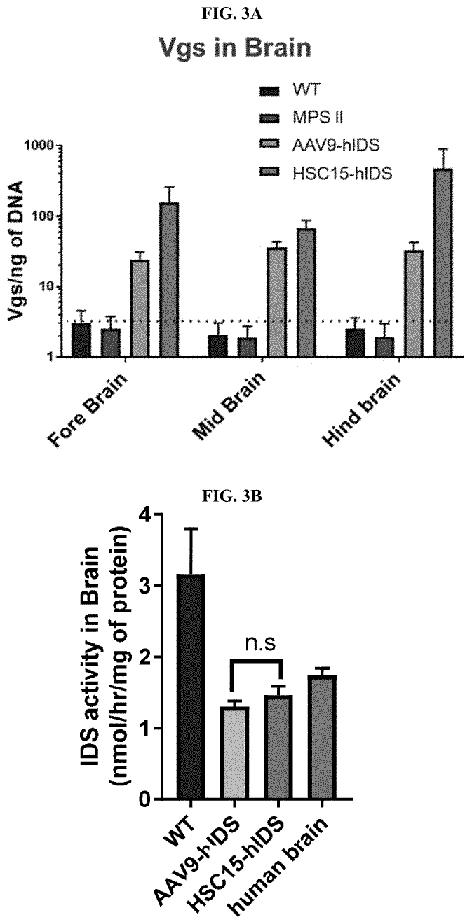

It was found that vector genomes and hI2S activity were detected in brain and liver tissues of Ids KO hemi mice. FIG. 2 shows the vector genomes (FIG. 2A) and I2S activity (FIG. 2B) detected in the liver of wild-type, Ids KO hemi males, or Ids KO hemi males administered the rAAV as indicated. * indicates statistical significance at p<0.05; *** indicates statistical significance at p<0.001, and **** indicates statistical significance at p<0.0001, as compared to WT. FIG. 3 shows the vector genomes (FIG. 3A) and hI2S activity (FIG. 3B) detected in the brain of wild-type, Ids KO hemi males, or Ids KO hemi males administered the rAAV as indicated. It was found that the amount of vector genomes as well as hI2S activity were higher in the liver compared to the brain. In the brain, vector genome levels were found to be similar across the rostro-caudal axis and appears to be higher in Ids KO hemi mice administered pHM-05205 packaged in AAVHSC15 capsid compared to Ids KO hemi mice administered pHM-05205 packaged in AAV9 capsid.

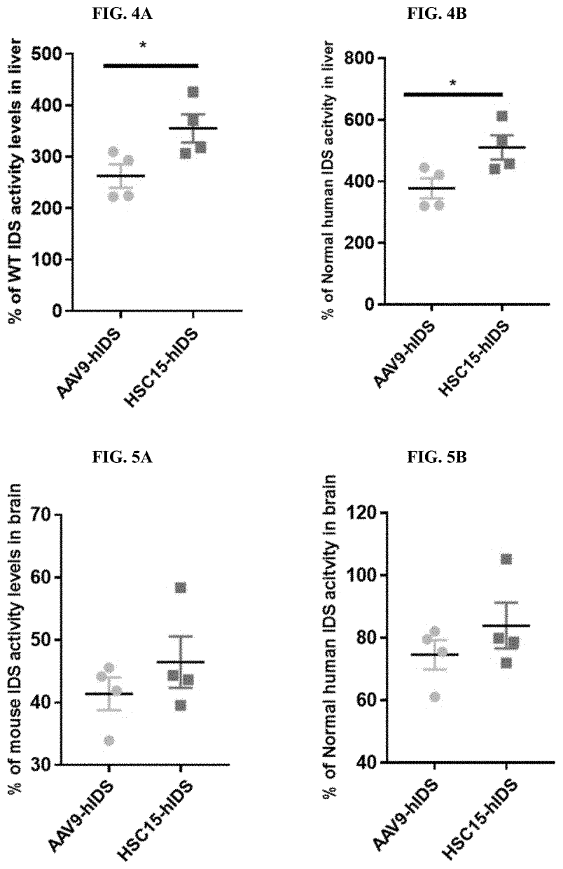

FIG. 4 shows hI2S activity in the liver and brain of Ids KO hemi mice administered pHM-05205 packaged in AAVHSC15 capsid compared to Ids KO hemi mice administered pHM-05205 packaged in AAV9 capsid. It was found that hI2S activity levels detected in the liver were supraphysiologic for both Ids KO hemi mice administered pHM-05205 packaged in AAVHSC15 capsid compared to Ids KO hemi mice administered pHM-05205 packaged in AAV9 capsid (FIG. 4A shows I2S activity as a percentage of wild-type I2S activity levels in liver; FIG. 4B shows I2S activity as a percentage of normal human I2S activity in liver). It was also found that Ids KO hemi mice administered pHM-05205 packaged in AAVHSC15 capsid exhibited significantly higher hI2S activity compared to Ids KO hemi mice administered pHM-05205 packaged in AAV9 capsid. In the brain, it was found that hI2S activity levels of Ids KO hemi mice administered pHM-05205 packaged in AAV9 capsid compared to Ids KO hemi mice administered pHM-05205 packaged in AAVHSC15 capsid were about 40% and about 45% of wild-type mouse, and about 75% and about 82% of adult human levels, respectively (FIG. 5A shows I2S activity as a percentage of mouse I2S activity levels in brain; FIG. 5B shows I2S activity as a percentage of normal human I2S activity in brain). * indicates statistical significance at p<0.05.

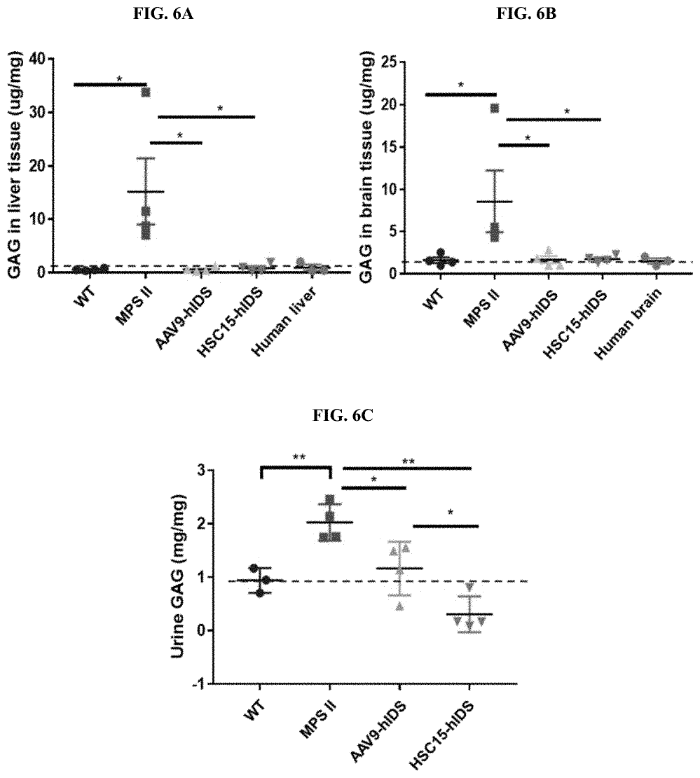

It was found that Ids KO hemi mice administered pHM-05205 packaged in AAVHSC15 capsid and Ids KO hemi mice administered pHM-05205 packaged in AAV9 capsid reduced GAG levels in the brain, liver, and urine compared to untreated Ids KO hemi mice (Ids KO hemi mice treated with vehicle). FIG. 6 shows the GAG levels in the liver (FIG. 6A), brain (FIG. 6B), and urine (FIG. 6C) of wild-type (WT), Ids KO hemi mice (MPS II), Ids KO hemi mice administered pHM-05205 packaged in AAV9 capsid (AAV9-hIDS), Ids KO hemi mice administered pHM-05205 packaged in AAVHSC15 capsid (HSC15-hIDS), and/or representative human. It was found that GAG levels in the liver and brain of Ids KO hemi mice administered pHM-05205 packaged in AAVHSC15 capsid and Ids KO mice administered pHM-05205 packaged in AAV9 capsid were reduced to wild-type levels. In the urine, GAG levels of Ids KO hemi mice administered pHM-05205 packaged in AAVHSC15 capsid were found to be significantly lower than in wild-type mice. * indicates statistical significance at p<0.05, and ** indicates statistical significance at p<0.01.

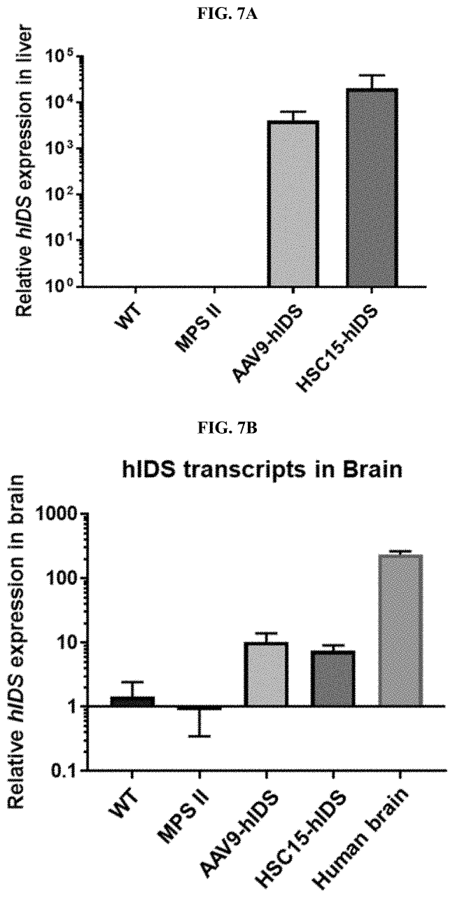

FIG. 7 shows that mRNA expression of hIDS was detected in the liver (FIG. 7A) and brain (FIG. 7B) of wild-type (WT), Ids KO hemi mice (MPS II), Ids KO hemi mice administered pHM-05205 packaged in AAV9 capsid (AAV9-hIDS), Ids KO hemi mice administered pHM-05205 packaged in AAVHSC15 capsid (HSC15-hIDS), and/or representative human.

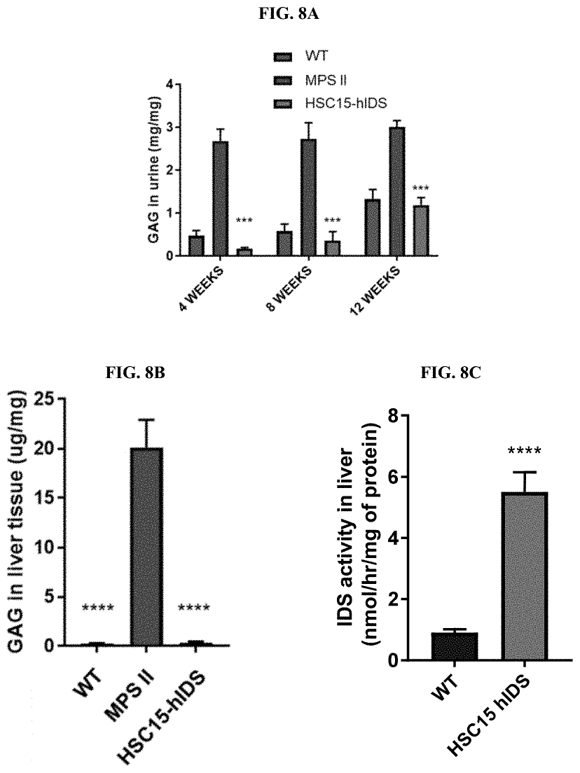

In liver tissue and urine at 12 weeks post-dosing, Ids KO hemi mice administered pHM-05205 packaged in AAVHSC15 capsid showed durability and rescue of phenotype. FIG. 8A shows that GAG levels in urine samples at the times as indicated were rescued to wild-type levels: wild-type mice (WT), Ids KO hemi mice (MPS II), and Ids KO hemi mice administered pHM-05205 packaged in AAVHSC15 capsid (HSC15-hIDS). *** indicates statistical significance at p<0.001. FIG. 8B shows that GAG levels in liver were rescued to wild-type levels: wild-type mice (WT), Ids KO hemi mice (MPS II), and Ids KO hemi mice administered pHM-05205 packaged in AAVHSC15 capsid (HSC15-hIDS). **** indicates statistical significance at p<0.0001. FIG. 8C shows that I2S activity in liver was increased: wild-type mice (WT), and Ids KO hemi mice administered pHM-05205 packaged in AAVHSC15 capsid (HSC15-hIDS). **** indicates statistical significance at p<0.0001. In addition, it was found that Ids KO hemi mice administered pHM-05205 packaged in AAVHSC15 reduced LAMP1 in the liver tissue as detected by immunohistochemistry using an anti-LAMP1 antibody.

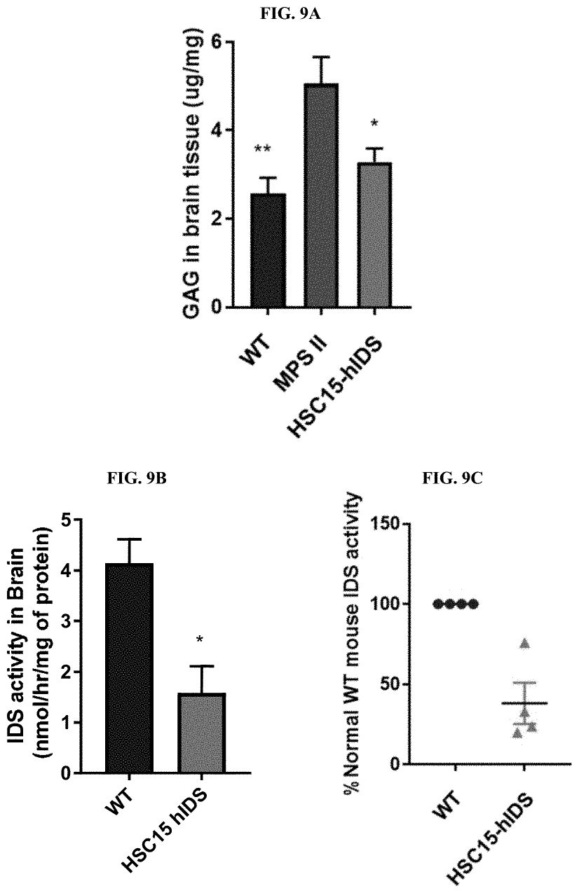

In the brain at 12 weeks post-dosing, Ids KO hemi mice administered pHM-05205 packaged in AAVHSC15 capsid showed durability and rescue in phenotype. FIG. 9A shows that GAG levels in the brain were rescued to wild-type levels: wild-type mice (WT), Ids KO hemi mice (MPS II), and Ids KO hemi mice administered pHM-05205 packaged in AAVHSC15 capsid (HSC15-hIDS). * indicates statistical significance at p<0.05, and ** indicates statistical significance at p<0.01. hI2S activity was detected in brain of wild type mice (WT) and Ids KO hemi mice administered pHM-05205 packaged in AAVHSC15 capsid (FIG. 9B and FIG. 9C). * indicates statistical significance at p<0.05.

### Example 3: Human IDS Transfer Vectors

This example provides human IDS transfer vectors T-004, T-005, and T-006 for expression of human IDS (hIDS) in a cell (e.g., a human cell or a mouse cell) into which the vector is transduced.

### a) T-004

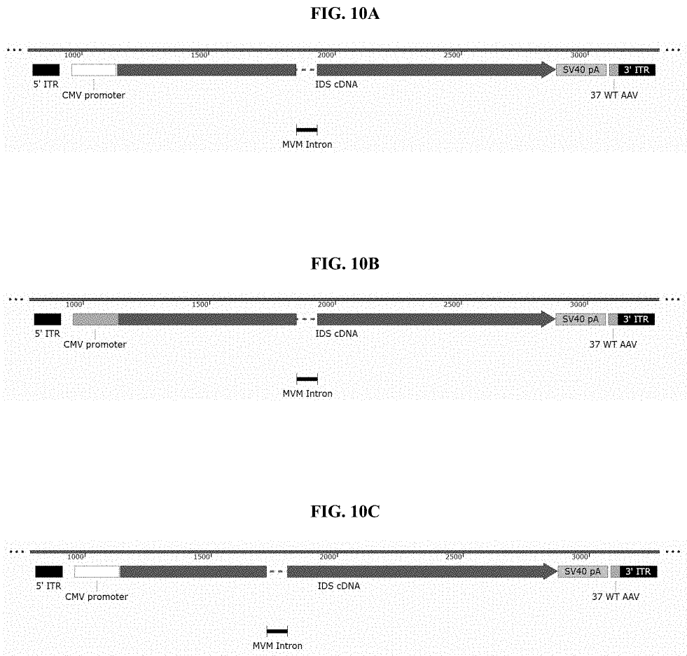

IDS transfer vector T-004, as shown in FIG. 10A, comprises 5′ to 3′ the following genetic elements: a 5′ ITR element; a transcriptional regulatory element comprising a CMV promoter; a silently-altered human IDS intron-inserted coding sequence; an SV40 polyadenylation sequence; and a 3′ ITR element. The sequences of these elements are set forth in Table 2. This vector is capable of expressing a human IDS protein in a cell (e.g., a human cell or a mouse cell) into which the vector is transduced.

### b) T-005

IDS transfer vector T-005, as shown in FIG. 10B, comprises 5′ to 3′ the following genetic elements: a 5′ ITR element; a transcriptional regulatory element comprising a CMV promoter; a silently-altered human IDS intron-inserted coding sequence; an SV40 polyadenylation sequence; and a 3′ ITR element. The sequences of these elements are set forth in Table 2. This vector is capable of expressing a human IDS protein in a cell (e.g., a human cell or a mouse cell) into which the vector is transduced.

### c) T-006

IDS transfer vector T-006, as shown in FIG. 10C, comprises 5′ to 3′ the following genetic elements: a 5′ ITR element; a transcriptional regulatory element comprising a CMV promoter; a silently-altered human IDS intron-inserted coding sequence; an SV40 polyadenylation sequence; and a 3′ ITR element. The sequences of these elements are set forth in Table 2. This vector is capable of expressing a human IDS protein in a cell (e.g., a human cell or a mouse cell) into which the vector is transduced.

TABLE 2
Genetic elements in human IDS transfer
vectors T-004, T-005, and T-006

| Genetic | T-004 | T-005 | T-006 |
|---|---|---|---|
| element | SEQ ID NO: | | |
| 5′ ITR element | 49 | 49 | 49 |
| Transcriptional | 29 | 29 | 29 |
| regulatory element | | | |
| Human IDS coding | 59 | 60 | 27 |
| sequence | | | |
| SV40 | 45 | 45 | 45 |
| polyadenylation | | | |
| sequence | | | |
| 3′ ITR element | 14 | 14 | 14 |
| rAAV genome (from | 61 | 63 | 65 |
| promoter to polyA | | | |
| sequence) | | | |
| rAAV genome (from | 62 | 64 | 66 |
| 5′ ITR to 3′ ITR) | | | |

The vectors disclosed herein can be packaged in an AAV capsid, e.g., an AAV Glade F capsid, such as, without limitation, an AAVHSC5, AAVHSC7, AAVHSC15, or AAVHSC17 capsid. The packaged viral particles can be administered to a wild-type animal, or an IDS-deficient animal.

### Example 4: IDS Gene Transfer in a Mucopolysaccharidosis (MPS) II (Hunter Syndrome) Mouse Model

In this example, wild-type and Ids KO hemizygous (Ids KO hemi; also referred to as MPS II) male mice, 6-9 weeks of age, were used. A single dose of 2e13 vgs/kg of pHM-05205, T-004, T-005, or T-006 packaged in either AAVHSC15 capsid or AAV9 capsid was administered intravenously to the mice. Mice were sacrificed 4 weeks post-dosing.

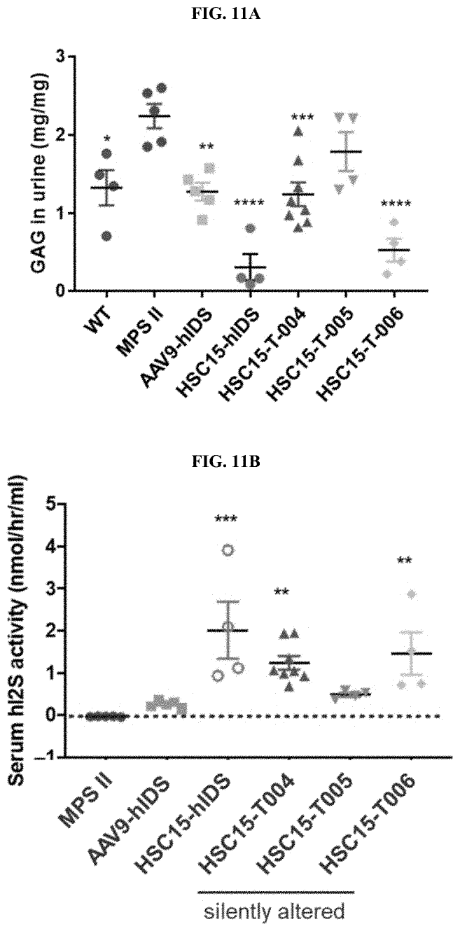

FIG. 11 shows the levels of GAG detected in urine (FIG. 11A) and serum I2S activity (FIG. 11B) of four wild type mice (WT); four Ids KO hemi mice (MPS II); four Ids KO hemi mice administered pHM-05205 packaged in AAV9 capsid (AAV9-hIDS); four Ids KO hemi mice administered pHM-05205 packaged in AAVHSC15 capsid (HSC15-hIDS); eight Ids KO hemi mice administered T-004 packaged in AAVHSC15 capsid (HSC15-T-004); four IDS KO hemi mice administered T-005 packaged in AAVHSC15 capsid (HSC15-T-005); and four IDS KO hemi mice administered T-006 packaged in AAVHSC15 capsid (hIDS-T-006). As shown in FIG. 11A, GAG levels in urine of treated Ids KO hemi mice were reduced compared to untreated Ids KO hemi mice (Ids KO hemi mice treated with vehicle). As shown in FIG. 11B, serum I2S activity was detectable in Ids KO hemi mice administered T-004, T-005, or T-006 packaged in AAVHSC15 capsid. * indicates statistical significance at p<0.05, ** indicates statistical significance at p<0.01, *** indicates statistical significance at p<0.001, and **** indicates statistical significance at p<0.0001.

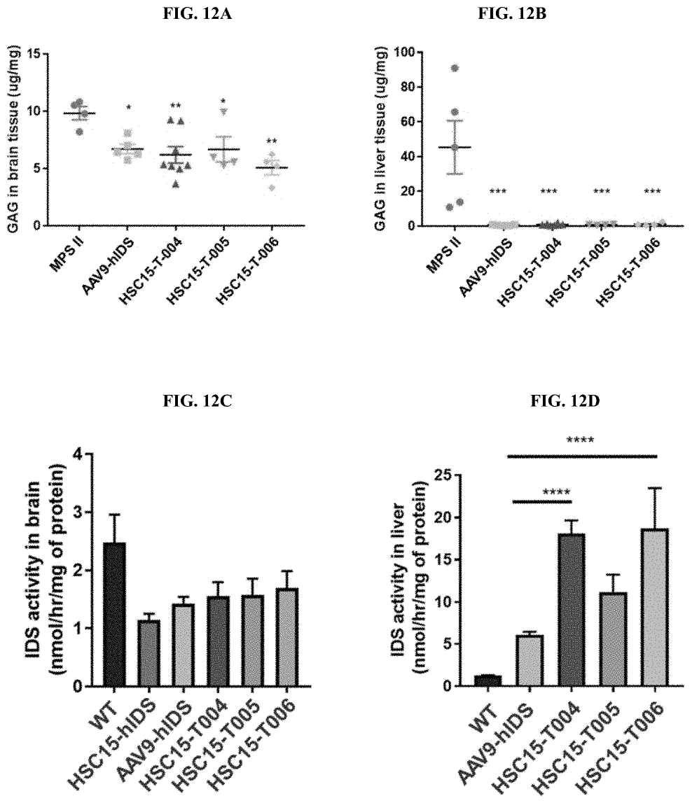

FIG. 12 shows the levels of GAG detected in brain and liver (FIG. 12A and FIG. 12B) and I2S activity in brain and liver (FIG. 12C and FIG. 12D) of wild type mice (WT); Ids KO hemi mice (MPS II); Ids KO hemi mice administered pHM-05205 packaged in AAV9 capsid (AAV9-hIDS); Ids KO hemi mice administered pHM-05205 packaged in AAVHSC15 capsid (HSC15-hIDS); Ids KO hemi mice administered T-004 packaged in AAVHSC15 capsid (HSC15-T-004); Ids KO hemi mice administered T-005 packaged in AAVHSC15 capsid (HSC15-T-005); and/or Ids KO hemi mice administered T-006 packaged in AAVHSC15 capsid (hIDS-T-006). As shown, GAG levels in the brain (FIG. 12A) and liver (FIG. 12B) of treated Ids KO hemi mice were reduced compared to untreated Ids KO hemi mice (Ids KO hemi mice treated with vehicle). As shown, I2S activity in the brain (FIG. 12C) and liver (FIG. 12D) was detectable in Ids KO hemi mice administered T-004, T-005, or T-006 packaged in AAVHSC15 capsid. * indicates statistical significance at p<0.05, ** indicates statistical significance at p<0.01, *** indicates statistical significance at p<0.001, and **** indicates statistical significance at p<0.0001.

### Example 5: IDS Gene Transfer in a Mucopolysaccharidosis (MPS) II (Hunter Syndrome) Mouse Model

In this example, wild-type and Ids KO hemizygous (Ids KO hemi; also referred to as MPS II) males, 7-10 weeks of age, were used. A dose range comprising 2.2e13 vgs/kg, 6.5e13 vgs/kg, and 1.1e14 vgs/kg of pHM-05217 packaged in AAVHSC15 capsid was administered intravenously to the mice, 5 mice per group. Mice were sacrificed 4 weeks post-dosing. In this example, untreated mice refers to mice administered vehicle.

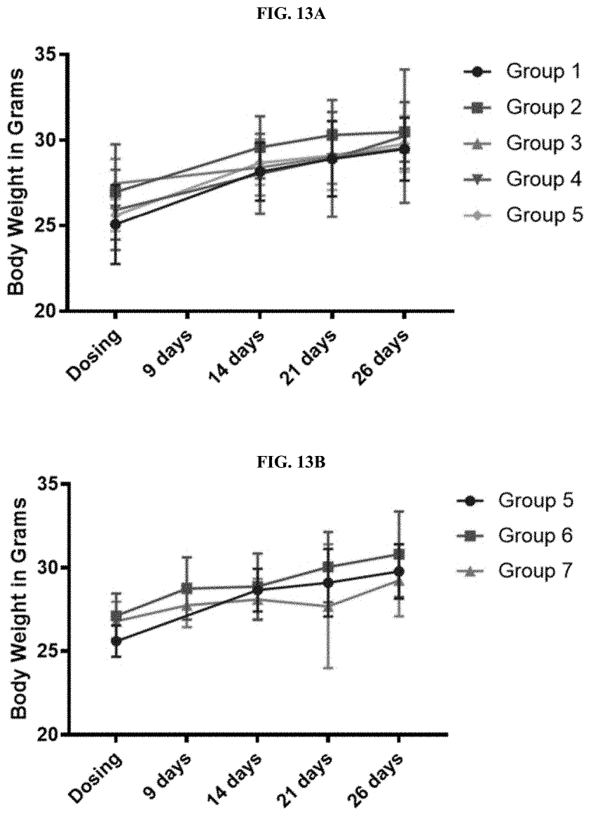

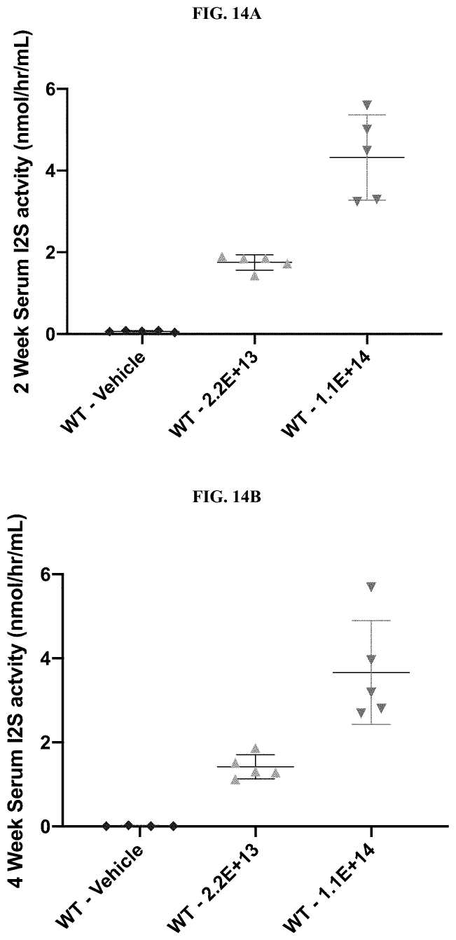

To investigate the safety of pHM-05217 packaged in AAVHSC15 capsid, the effects of administration of the virus to wild-type mice was studied. Tolerability of pHM-05217 packaged in AAVHSC15 capsid was demonstrated when no evidence of body weight decline was observed across dosages and over time (FIG. 13A and FIG. 13B). As shown in FIG. 13A and FIG. 13B, both wild-type and Ids KO hemi mice treated with pHM-05217 packaged in AAVHSC15 showed no evidence of decrease in body weight over time. In FIG. 13A, Group 1: Ids KO hemi mice control; Group 2: Ids KO hemi mice administered pHM-05217 packaged in AAVHSC15 capsid at a dose of 2.2e13 vgs/kg; Group 3: Ids KO hemi mice administered pHM-05217 packaged in AAVHSC15 capsid at a dose of 6.5e13 vgs/kg; Group 4: Ids KO hemi mice administered pHM-05217 packaged in AAVHSC15 capsid at a dose of 1.1e14 vgs/kg; and Group 5: wild-type mice. In FIG. 13B, Group 5: wild-type mice; Group 6 wild-type mice administered pHM-05217 packaged in AAVHSC15 capsid at a dose of 2.2e13 vgs/kg; and Group 7: wild-type mice administered pHM-05217 packaged in AAVHSC15 capsid at a dose of 1.1e14 vgs/kg.

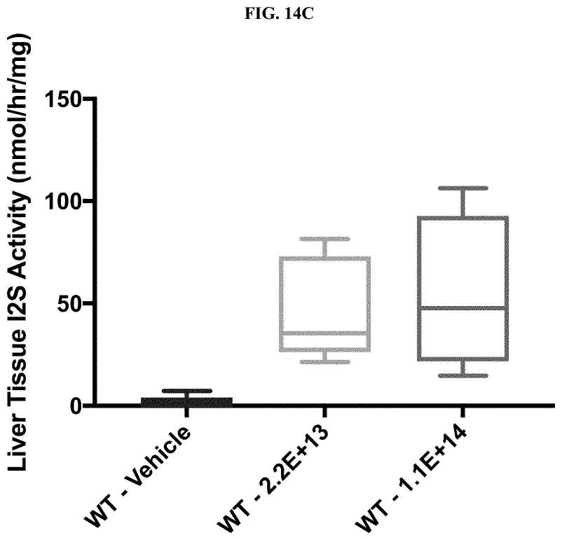

I2S activity in wild-type mice was found to be dose-dependent upon administration of pHM-05217 packaged in AAVHSC15 capsid. In the serum, at two weeks (FIG. 14A) and four weeks (FIG. 14B) post-dosing, wild-type mice administered pHM-05217 packaged in AAVHSC15 capsid at the doses as indicated exhibited a dose-dependent increase in I2S activity. Untreated wild-type (WT) mice and Ids KO hemizygous (MPS II) mice were used as controls. In the liver, at four weeks post-dosing (FIG. 14C), wild-type mice administered pHM-05217 packaged in AAVHSC15 capsid at the doses as indicated exhibited a dose-dependent increase in I2S activity. This demonstrated that human I2S activity is detectable in wild-type mice, and that increasing I2S activity in wild-type mice over normal levels does not affect body weight.

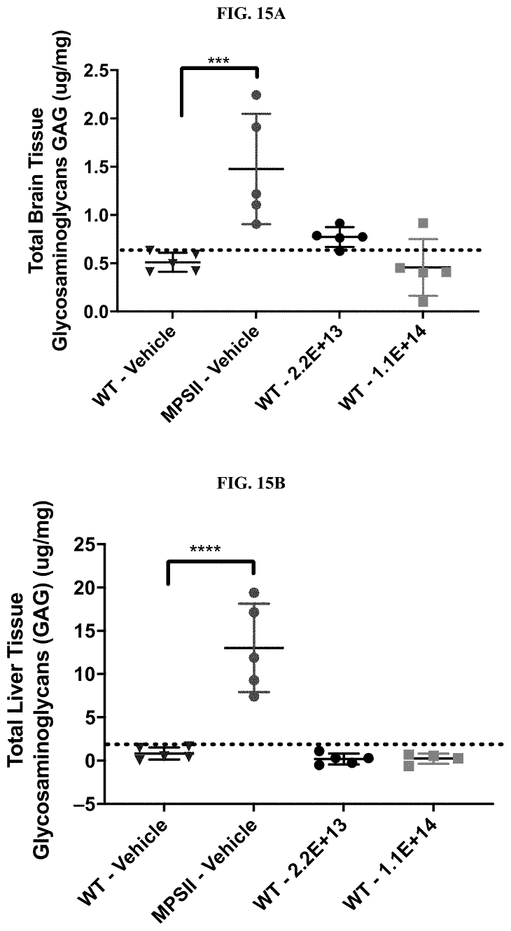

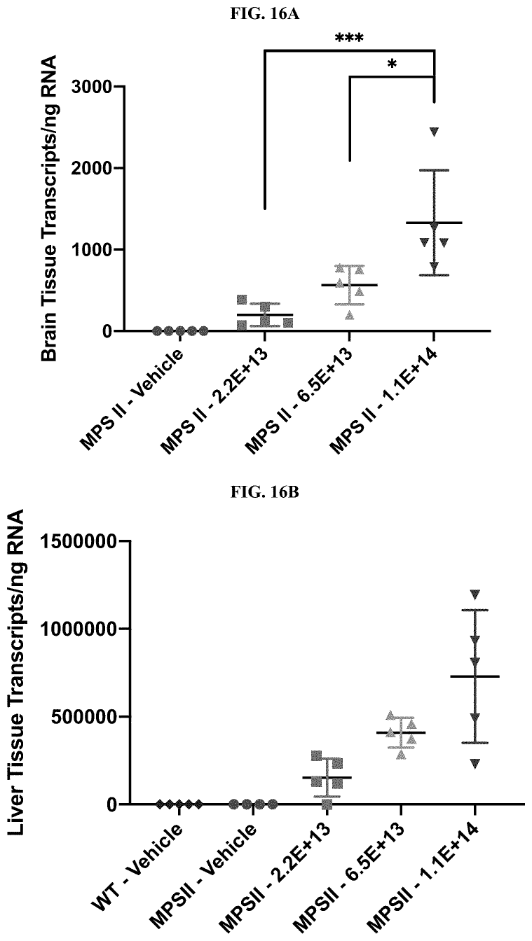

GAG levels in wild-type mice administered pHM-05217 packaged in AAVHSC15 capsid are similar to that of wild-type untreated mice and were not found to be further reduced below wild-type levels. In the brain (FIG. 15A) and the liver (FIG. 15B), GAG levels of treated Ids KO hemi mice were found to be comparable to wild-type untreated mice (controls). *** indicates statistical significance at p<0.001, and **** indicates statistical significance at p<0.0001.

It was found that expression in the brain and liver is dose-dependent upon administration of pHM-05217 packaged in AAVHSC15. FIG. 16A shows brain expression of Ids KO hemi mice administered pHM-05217 packaged in AAVHSC15 at the indicated doses, demonstrating an increase in expression with increasing dose. FIG. 16B shows liver expression of Ids KO hemi mice administered pHM-05217 packaged in AAVHSC15 at the indicated doses, demonstrating an increase in expression with increasing dose. In general, it was found that the liver had a higher amount of silently altered IDS expression than the brain. * indicates statistical significance at p<0.05, and *** indicates statistical significance at p<0.001.

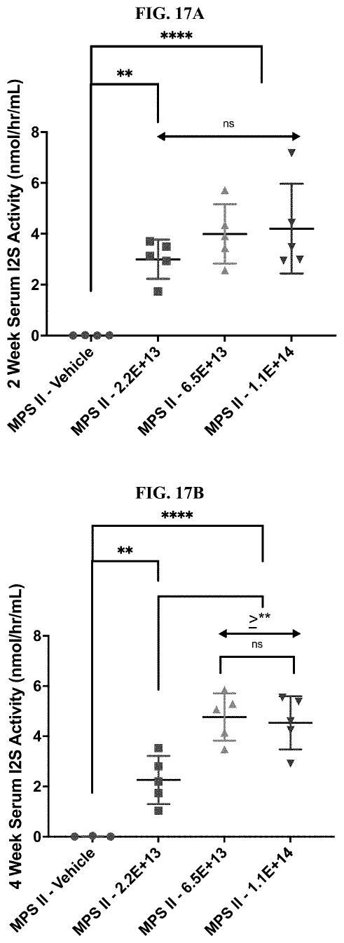

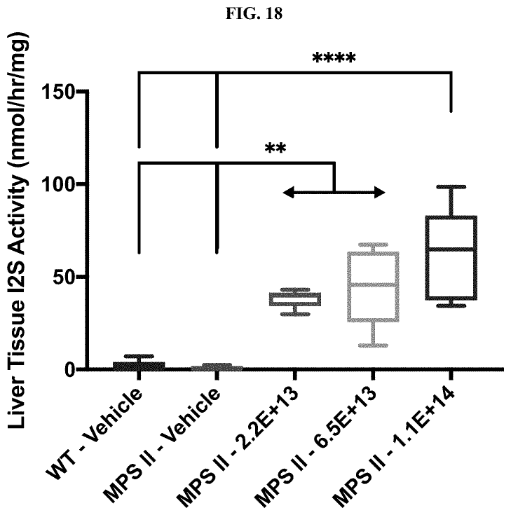

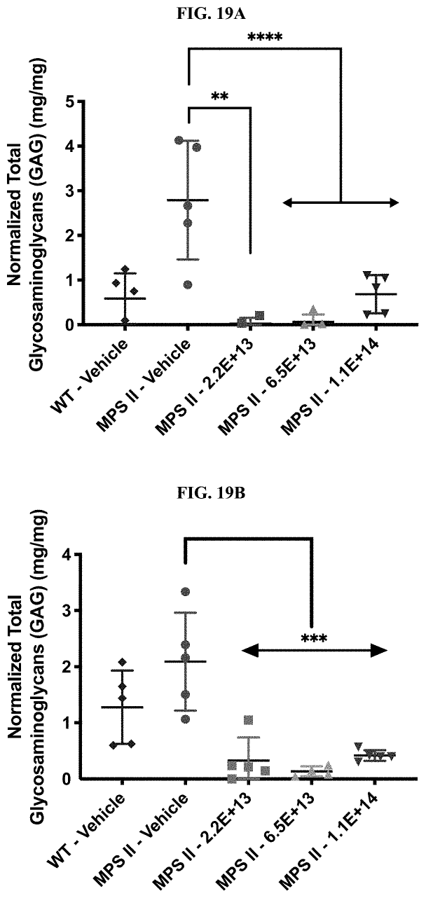

To investigate the efficacy of pHM-05217 packaged in AAVHSC15 capsid, the effects of administration of the virus to Ids KO hemi mice was studied. Serum I2S activity in Ids KO hemi mice administered pHM-05217 packaged in AAVHSC15 capsid was detected at two weeks (FIG. 17A) and remained consistent at four weeks post-dosing (FIG. 17B). At four weeks, serum I2S activity was found to be dose-dependent up to a dose of 6.5e13 vgs/kg. ** indicates statistical significance at p<0.01, and **** indicates statistical significance at p<0.0001.

pHM-05217 packaged in AAVHSC15 capsid also showed dose-dependent I2S activity in liver. FIG. 18 shows liver I2S activity of Ids KO hemi mice administered pHM-05217 packaged in AAVHSC15 capsid. ** indicates statistical significance at p<0.01, and **** indicates statistical significance at p<0.0001.

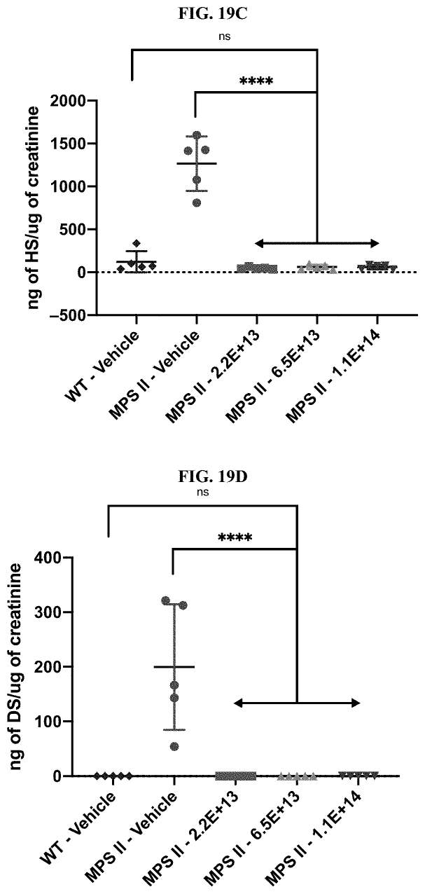

GAG levels in urine of Ids KO hemi mice administered pHM-05217 packaged in AAVHSC15 were found to be reduced to wild-type levels by all doses at two weeks (FIG. 19A) and four weeks post-dosing (FIG. 19B). GAG heparin sulphate (GAG-HS) (FIG. 19C) and GAG dermatan sulfate (GAG-DS) (FIG. 19D) levels in urine of Ids KO hemizygous mice administered pHM-05217 packaged in AAVHSC15 were found to be reduced to wild-type levels at four weeks post-dosing. GAG levels were determined by mass spectrometry and normalized to creatinine levels in each urine sample. Statistical analysis was performed using a two-way analysis of variance (ANOVA): ns indicates no statistical significance, ** indicates statistical significance at p<0.01, *** indicates statistical significance at p<0.001, and **** indicates statistical significance at p<0.0001.

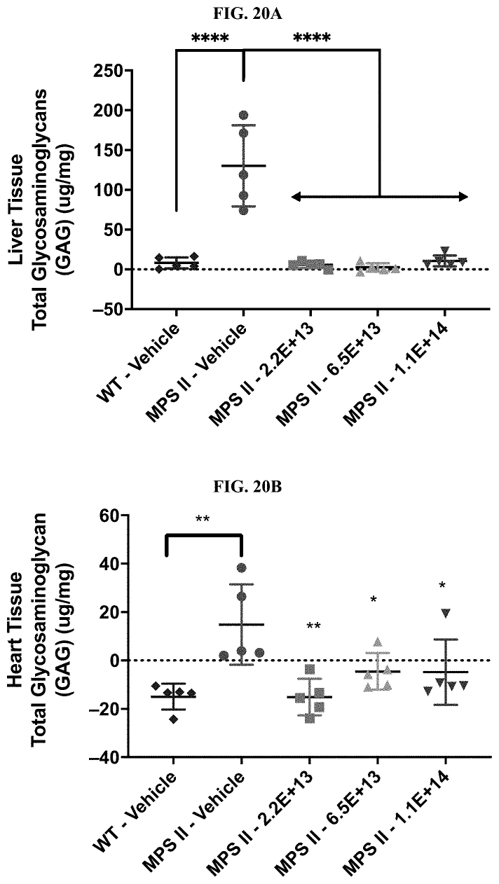

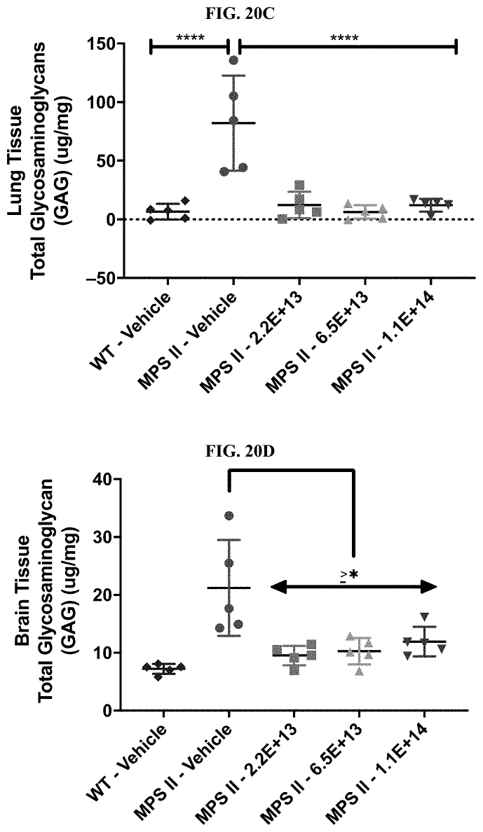

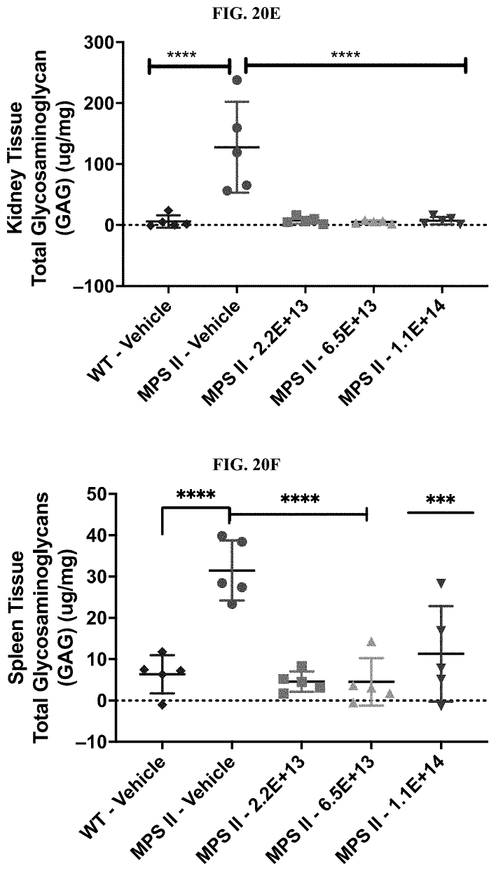

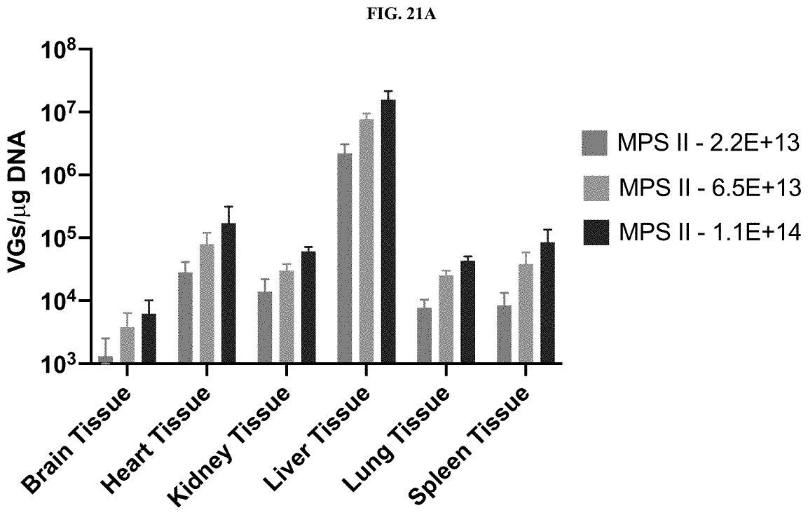

GAG levels in liver (FIG. 20A), heart (FIG. 20B), lung (FIG. 20C), brain (FIG. 20D), kidney (FIG. 20E), and spleen (FIG. 20F) of Ids KO hemi mice administered pHM-05217 packaged in AAVHSC15 were found to be reduced to wild-type levels by all doses at four weeks post-dosing. * indicates statistical significance at p<0.05, ** indicates statistical significance at p<0.01, *** indicates statistical significance at p<0.001, and **** indicates statistical significance at p<0.0001.

### Example 6: IDS Gene Transfer in a Mucopolysaccharidosis (MPS) II (Hunter Syndrome) Mouse Model

In another example, a 4-week single-intravenous dose-range finding study in adult wild-type and Ids KO hemizygous mice (Ids KO hemi; also referred to as MPS II) was performed. 2.2e13 vgs/kg, 6.5e13 vgs/kg, and 1.1e14 vgs/kg of pHM-05217 packaged in AAVHSC15 capsid was administered intravenously to the mice, 4-5 mice per group. Mice were sacrificed 4 weeks post-dosing. In these experiments, pHM-05217 packaged in AAVHSC15 was found to cross the blood-brain barrier and transduce cells of the brain, leading to expression of I2S and significant heparan sulfate reduction and dose-dependent LAMP1 reduction in the brain. Serum and liver I2S activity was also found to be dose-dependent. At all doses, heparan sulfate levels were found to be reduced in all tested peripheral tissue. Doses of up to 1.1e14 vgs/kg of pHM-05217 packaged in AAVHSC15 were found to be tolerated, based on lack of body weight reduction in MPS II or WT treated animals.

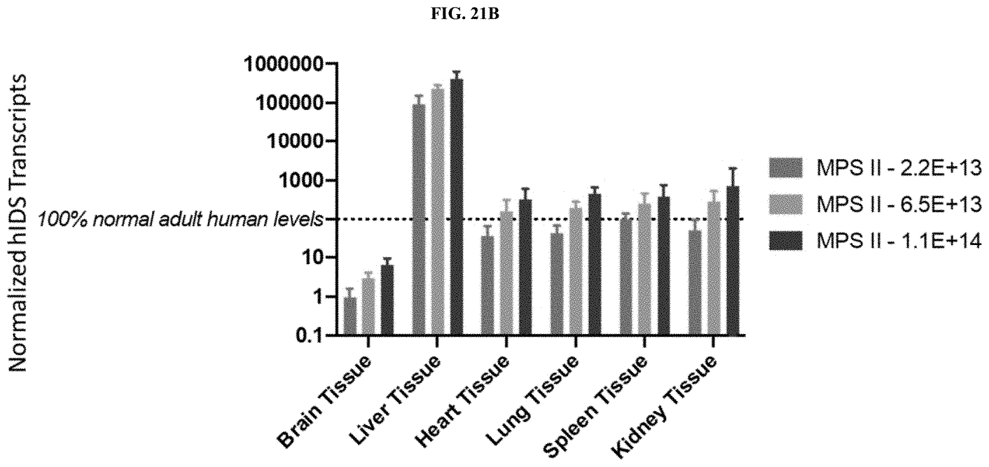

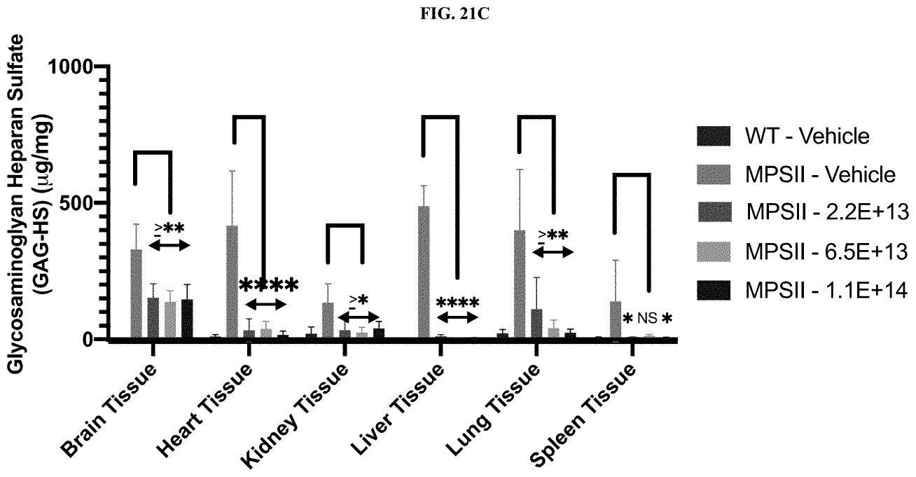

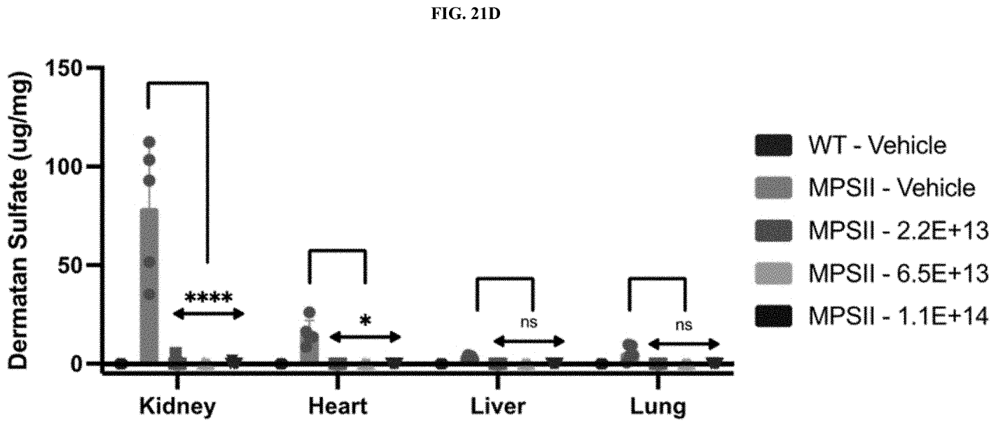

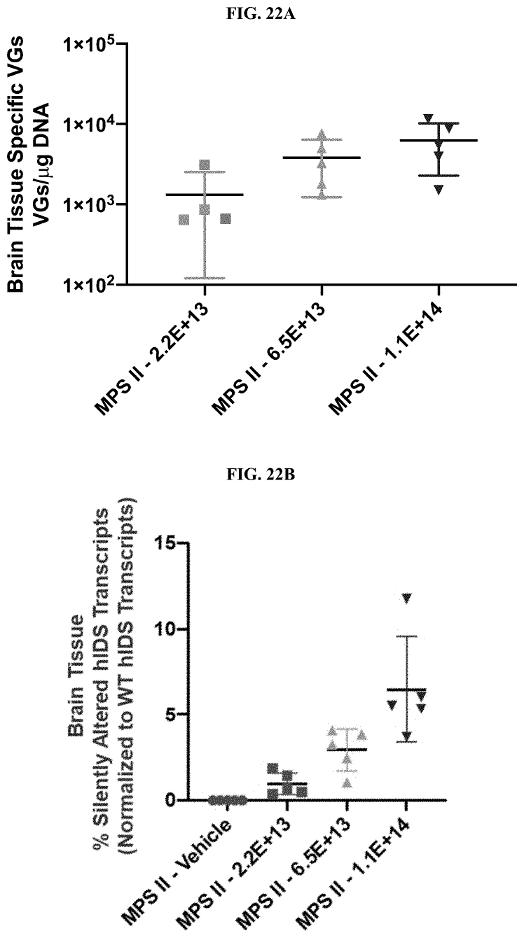

A single intravenous administration of pHM-05217 packaged in AAVHSC15 capsid was found to result in a dose-dependent increase in the level of vector genomes (FIG. 21A) and hIDS transcripts in key murine peripheral and central organs (FIG. 21B). FIG. 21B shows the percentage of silently altered hIDS transcripts normalized to wild-type hIDS transcripts. Heparan sulfate (FIG. 21C), dermatan sulfate (FIG. 21D), and/or total GAG levels were found to be reduced in all organs at all doses. In FIGS. 21C and 21D, * indicates statistical significance at p<0.05, ** indicates statistical significance at p<0.01, **** indicates statistical significance at p<0.0001, and ns indicates not significant.

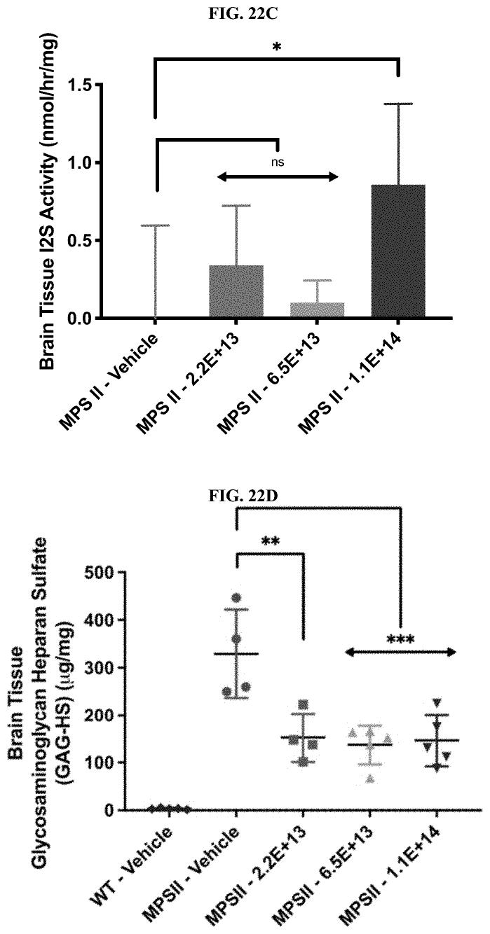

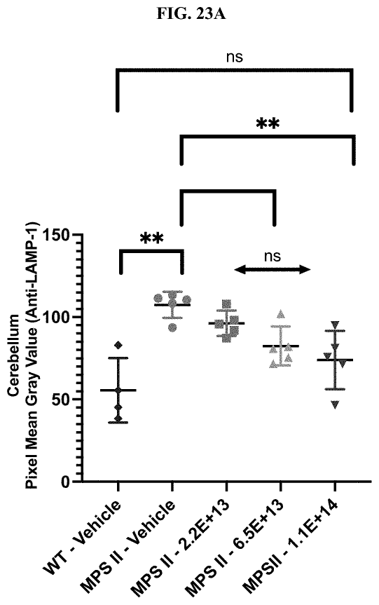

At 4-weeks post-dosing, MPS II mice administered pHM-05217 packaged in AAVHSC15 capsid exhibited a dose-dependent increase in the level of vector genomes (FIG. 22A), percentage of silently altered hIDS transcripts normalized to human wild-type hIDS transcripts (FIG. 22B), and I2S activity (FIG. 22C), in the brain. Heparan sulfate levels in the brains of MPS II mice administered pHM-05217 packaged in AAVHSC15 were found to be reduced by all doses at four-weeks post-dosing (FIG. 22D). As demonstrated in FIGS. 22A-22D, pHM-05217 packaged in AAVHSC15 capsid crossed the blood-brain barrier, transduced brain tissue, expressed silently altered hIDS, resulted in detectable I2S activity in the brain, and reduced brain tissue-specific GAGs. In FIGS. 22A-22D, * indicates statistical significance at p≤0.05, ** indicates statistical significance at p≤0.01, *** indicates statistical significance at p<0.001, and ns indicates not significant.

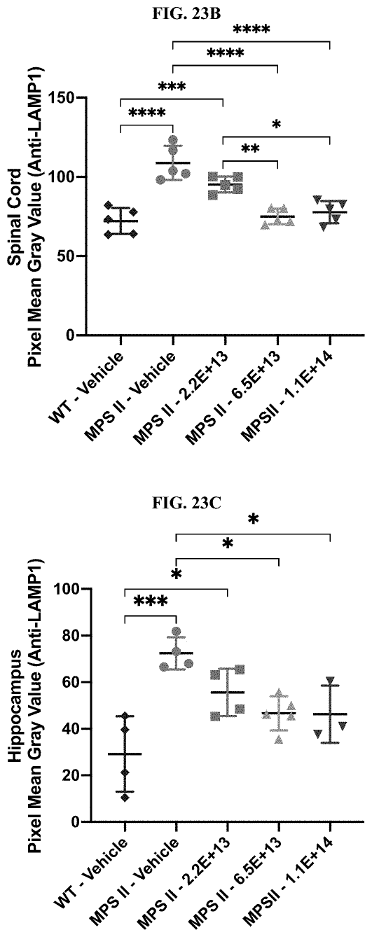

To further assess the effect of administration of pHM-05217 packaged in AAVHSC15 capsid on brain pathology, the cerebellum (FIG. 23A), spinal cord (FIG. 23B), and hippocampus (FIG. 23C) was assayed for lysosomal-associated membrane protein 1 (LAMP1) by immunohistochemistry (IHC). Presence of LAMP1 is evidence of lysosomal burden. Detection of LAMP1 by immunohistochemistry (IHC) was carried out using a rabbit polyclonal anti-LAMP1 antibody (Abcam, ab24170). Briefly, formalin fixed paraffin-embedded (FFPE) samples were sectioned at 4 μm or 6 μm and mounted onto charged slides. Slides were treated and processed using an autostainer and stained with anti-LAMP1 primary antibody for 30 minutes (0.25 μg/ml), and an anti-Rabbit Labelled Polymer-HRP for 30 minutes. Images of the stained sections were taken and the Pixel Mean Gray Value was analyzed, allowing for a semi-quantitative report of the expression of LAMP1 in each of the sections. As shown in FIGS. 23A-23C, pHM-05217 packaged in AAVHSC15 crossed the blood-brain barrier, and resulted in a dose-dependent trend in LAMP1 reduction in the CNS of treated MPS II mice. In FIGS. 23A-23C, * indicates statistical significance at p≤0.05, ** indicates statistical significance at p≤0.01, *** indicates statistical significance at p<0.001, **** indicates statistical significance at p≤0.0001, and ns indicates not significant.

LAMP1 expression was also analayzed by IHC in key organs including the liver, spleen, heart, kidney, and lung. Qualitative analysis of MPS II mice administered pHM-05217 packaged in AAVHSC15 capsid at a dose of 1.1e14 vgs/kg demonstrated that LAMP1 expression in the liver, spleen, heart, kidney, and lung of treated MPS II mice was reduced, compared to untreated MPS II mice (MPS II mice administered vehicle).

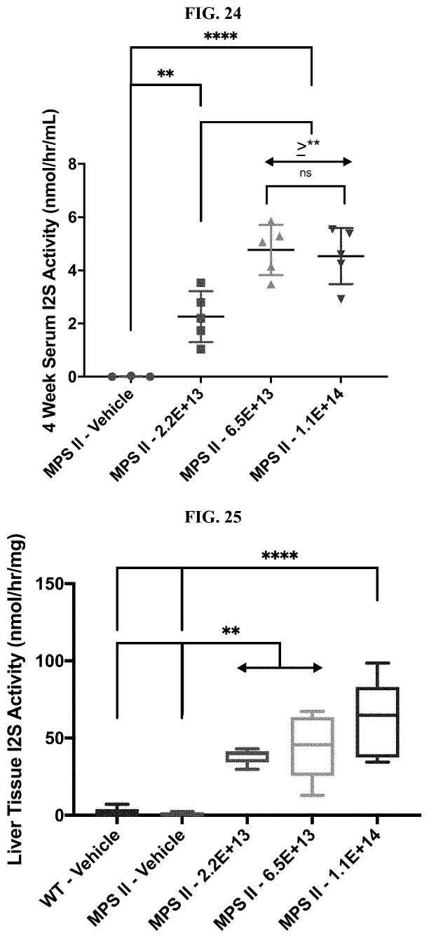

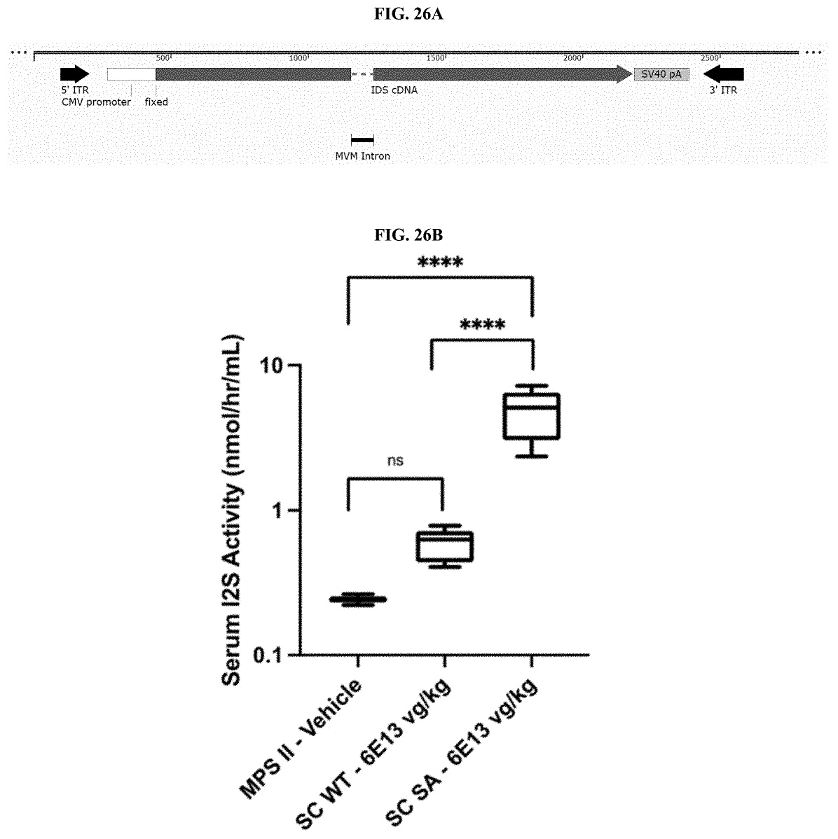

At four-weeks post-dosing, MPS II mice administered pHM-05217 packaged in AAVHSC15 capsid showed a dose-dependent increase in I2S activity in serum (FIG. 24) and in the liver (FIG. 25). In FIG. 24 and FIG. 25, ** indicates statistical significance at p≤0.01, **** indicates statistical significance at p<0.0001, and ns indicates not significant

### Example 7: Comparison Between Wild-Type and Silently Altered hIDS Transfer Vectors

This example provides human IDS transfer vector pHM-05205, for expression of human IDS (hIDS) in a cell (e.g., a human cell or a mouse cell) into which the vector is transduced. This example provides a comparison between the efficacy of hIDS transfer vectors T-006 and pHM-05205. T-006 is described in Example 3, and pHM-05205 is described below.

pHM-05205

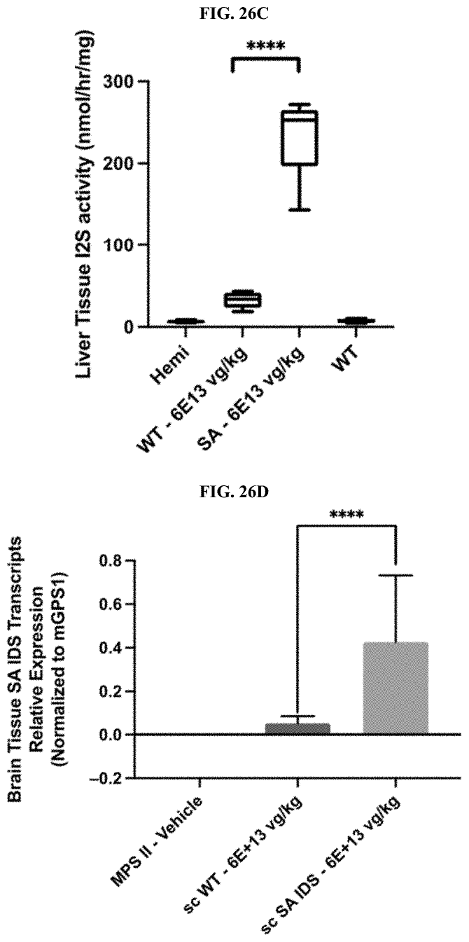

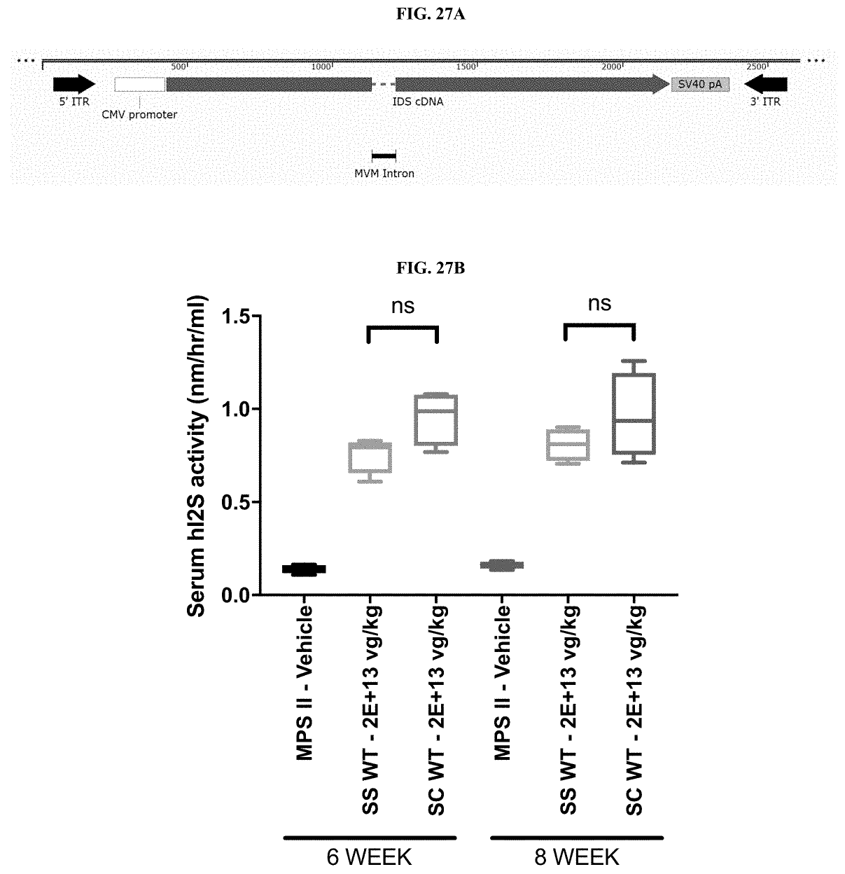

IDS transfer vector pHM-05205, as shown in FIG. 26A, comprises 5′ to 3′ the following genetic elements: a 5′ ITR element; a transcriptional regulatory element comprising a CMV promoter; a wild-type human IDS intron-inserted coding sequence; an SV40 polyadenylation sequence; and a 3′ ITR element. The sequences of these elements are set forth in Table 3. This vector is capable of expressing a human IDS protein in a cell (e.g., a human cell or a mouse cell) into which the vector is transduced.

TABLE 3
Genetic elements in human IDS transfer vector pHM-05205

| | pHM-05205 |
|---|---|
| Genetic element | SEQ ID NO: |
| 5′ ITR element | 49 |
| Transcriptional | 29 |
| regulatory element | |
| Human IDS coding | 25 |
| sequence | |
| SV40 | 45 |
| polyadenylation | |
| sequence | |
| 3′ ITR element | 14 |
| rAAV genome (from | 75 |
| promoter to polyA | |
| sequence) | |
| rAAV genome (from | 76 |
| 5′ ITR to 3′ ITR) | |

In order to test the efficacy of an hIDS transfer vector comprising a wild-type hIDS coding sequence (pHM-05205) and an hIDS transfer vector comprising a silently altered hIDS coding sequence (T-006), T-006 and pHM-05205 were each packaged in AAVHSC15 and administered to MPS II mice at a dose of 6e13 vgs/kg. Mice were sacrificed 4-weeks post-dosing and I2S activity in the serum (FIG. 26B) and liver (FIG. 26C) was measured, as well as the relative expression of hIDS transcripts normalized to the expression of murine G protein pathway suppressor 1 (GPS1) (FIG. 26D). As shown in FIGS. 26B and 26C, administration of the silently altered hIDS transfer vector (T-006; “SC SA”) resulted in significantly higher I2S activity in the serum and liver compared to administration of the wild-type hIDS transfer vector (pHM-05205; “SC WT”), respectively, in treated MPS II mice. FIG. 26D shows that administration of the silently altered hIDS transfer vector results in a significantly higher relative expression of hIDS transcripts in brain tissue compared to the administration of the wild-type hIDS transfer vector in treated MPS II mice. MPS II mice treated with vehicle were used as control. In FIGS. 26B-26D, **** indicates statistical significance at p≤0.0001, and ns indicates not significant.

### Example 8: Comparison Between Single-Stranded and Self-Complementary hIDS Transfer Vectors

This example provides human IDS transfer vector pHM-05211, for expression of human IDS (hIDS) in a cell (e.g., a human cell or a mouse cell) into which the vector is transduced. This example provides a comparison between hIDS transfer vectors pHM-05205 and pHM-05211. pHM-05205 is described in Example 7, and pHM-05211 is described below.

pHM-05211

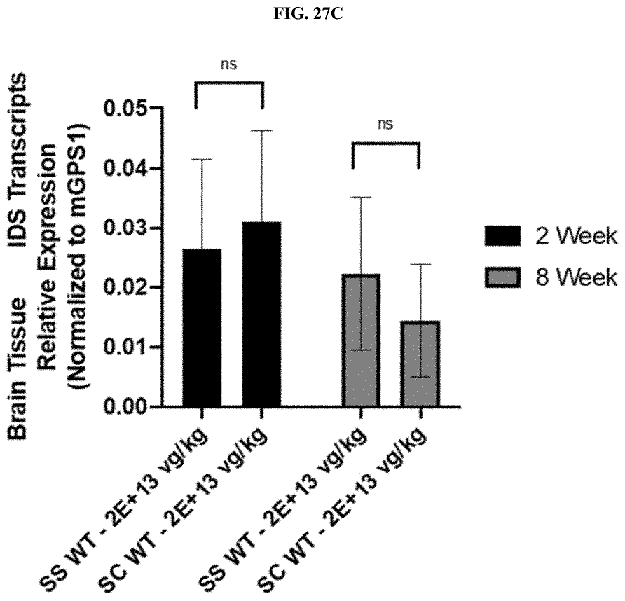

IDS transfer vector pHM-05211, as shown in FIG. 27A, comprises 5′ to 3′ the following genetic elements: a 5′ ITR element; a transcriptional regulatory element comprising a CMV promoter; a wild-type human IDS intron-inserted coding sequence; an SV40 polyadenylation sequence; and a 3′ ITR element. The sequences of these elements are set forth in Table 4. This vector is capable of expressing a human IDS protein in a cell (e.g., a human cell or a mouse cell) into which the vector is transduced.

TABLE 4
Genetic elements in human IDS transfer vector pHM-05211

| Genetic | pHM-05211 |
|---|---|
| element | SEQ ID NO: |
| 5′ ITR element | 18 |
| Transcriptional | 29 |
| regulatory element | |
| Human IDS coding | 25 |
| sequence | |
| SV40 | 45 |
| polyadenylation | |
| sequence | |
| 3′ ITR element | 14 |
| rAAV genome (from | 77 |
| promoter to polyA | |
| sequence) | |
| rAAV genome (from | 78 |
| 5′ ITR to 3′ ITR) | |

A comparison between a single-stranded hIDS transfer vector (pHM-05211; “SS WT”) and a self-complementary hIDS transfer vector (pHM-05205; “SC WT”) was performed. FIG. 27B shows the level of serum hI2S activity detected in MPS II mice administered pHM-05211 or pHM-05205, each packaged in AAVHSC15 capsid, at a dose of 2e13 vgs/kg. Serum hI2S activity was measured at 6 or 8 weeks post-dosing, as indicated. No significant difference was found between the ability of the single-stranded and self-complementary hIDS transfer vectors to induce serum hI2S activity. FIG. 27C shows the relative expression of hIDS transcripts normalized to the expression of murine G protein pathway suppressor 1 (GPS1) in MPS II mice treated with the single-stranded or self-complementary transfer vector. Mice were sacrificed at 2 or 8 weeks post-dosing, as indicated, and no difference between relative expression of hIDS transcripts was detected between single-stranded or self-complementary transfer vector-treated mice in each cohort. ns indicates not significant.

Analytical ultracentrifugation sedimentation velocity (AUC) is an analytical method used to quantify macromolecules based on sedimentation coefficients. For analysis of rAAV samples, AUC can be used to determine the relative percentage of DNA-containing (full and partially full capsids) and empty capsids. AUC profiles were determined for the single-stranded and self-complementary transfer vectors. The AUC profile of the single-stranded transfer vector demonstrated a profile with a higher percentage of full capsids compared to the AUC profile of the self-complementary transfer vector. The self-complementary transfer vector (pHM-05205) resulted in 31.7% fully packaged capsids and the single-stranded transfer vector (pHM-05211) resulted in 85.0% fully packaged capsids.

### Example 9: hIDS Gene Transfer in a Mucopolysaccharidosis (MPS) II (Hunter Syndrome) Mouse Model

This example describes a 52-week single-intravenous dose time course, durability, and efficacy study in adult wild-type and Ids KO hemizygous mice (Ids KO hemi; also referred to as MPS II mice). A 1.8e14 vgs/kg dose of pHM-05217 packaged in AAVHSC15 capsid was administered intravenously to the mice, 3-5 mice per group.

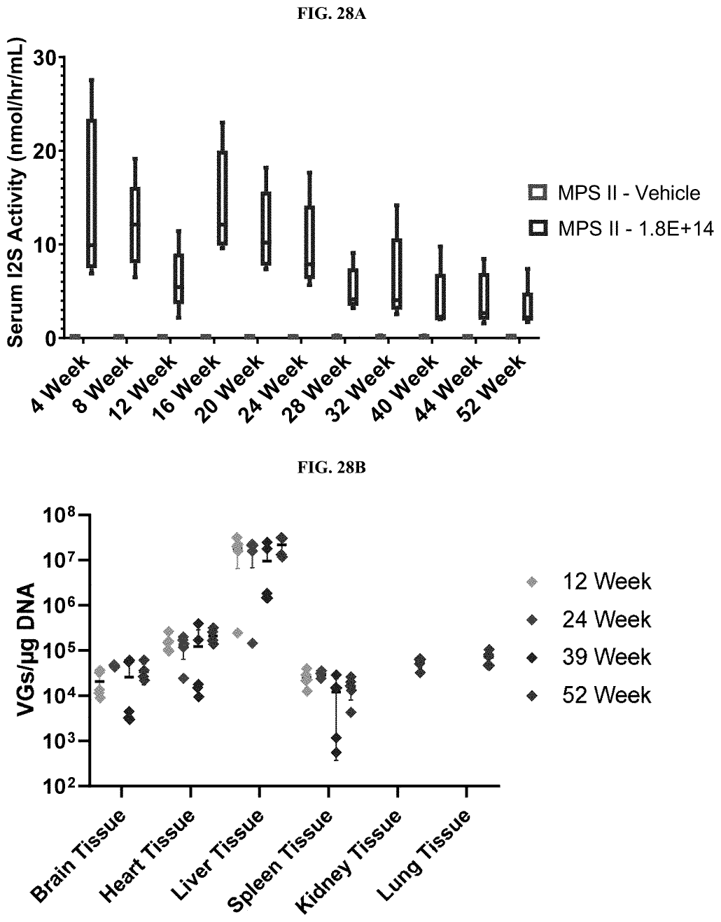

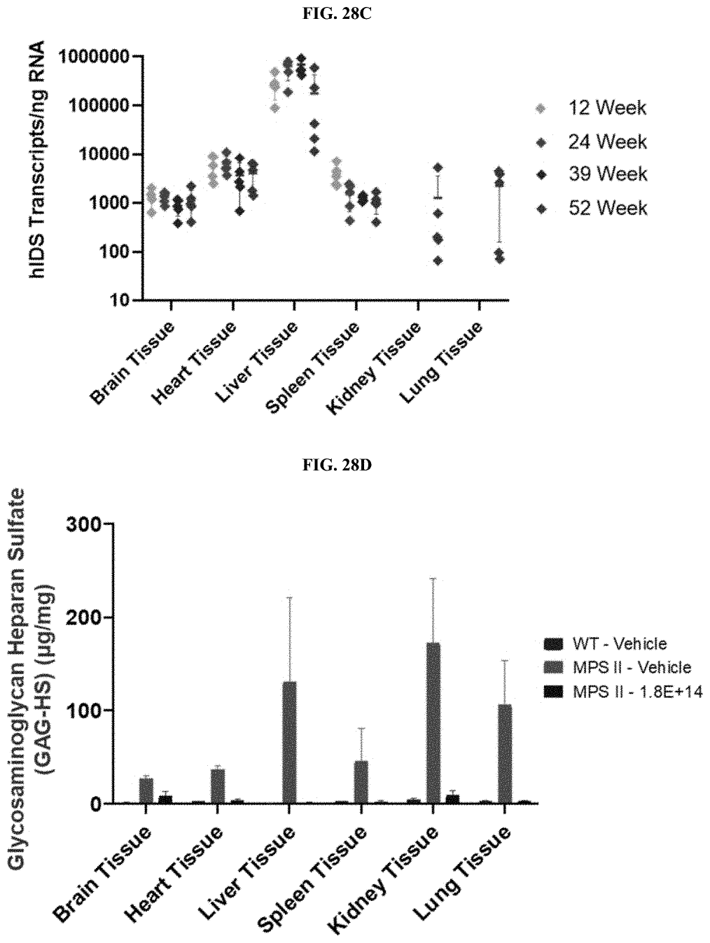

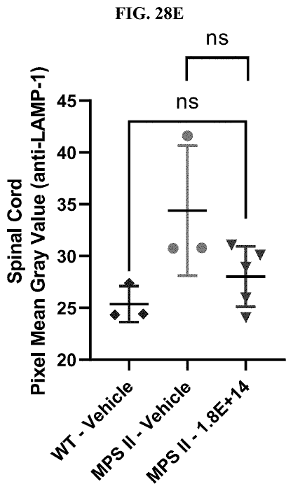

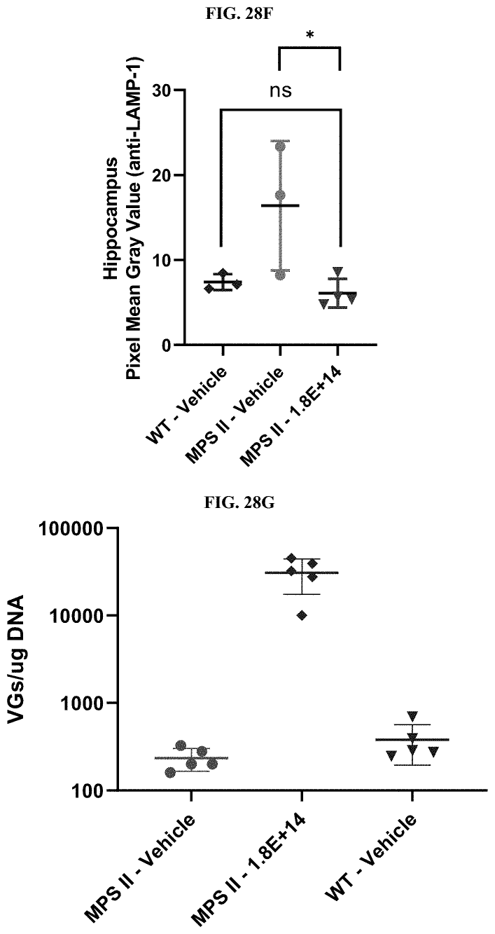

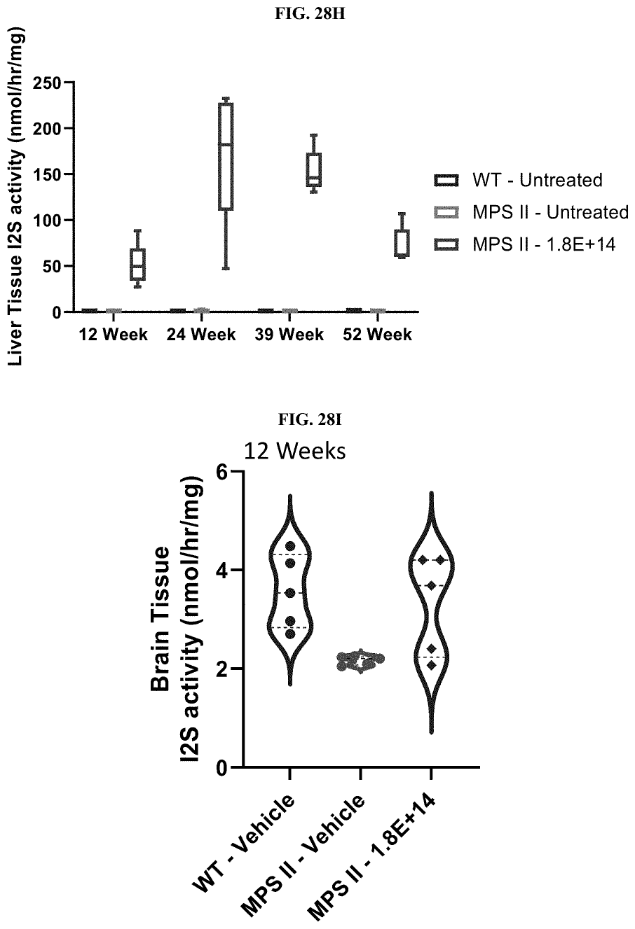

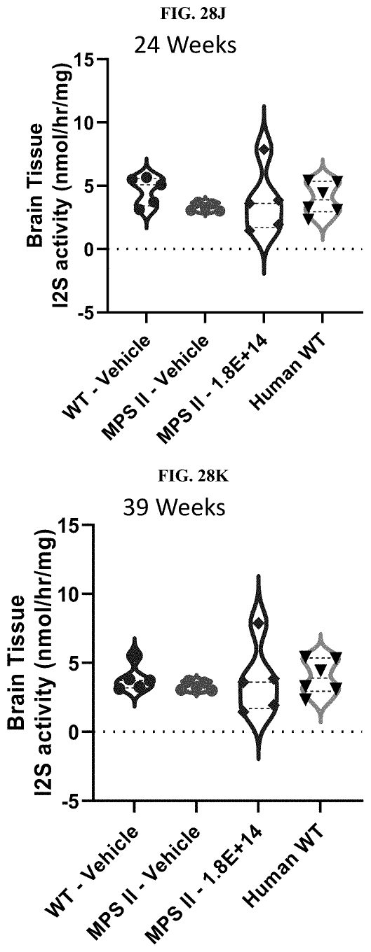

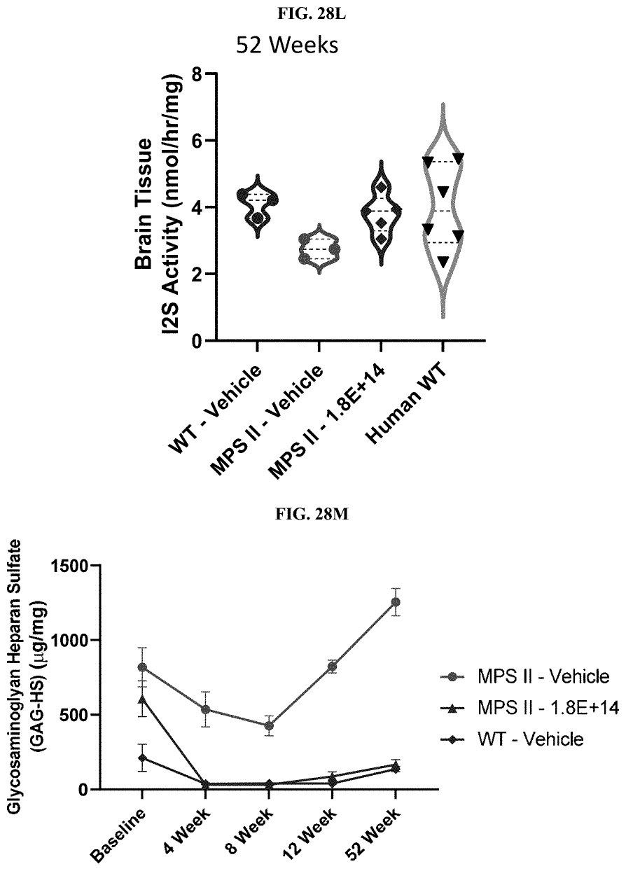

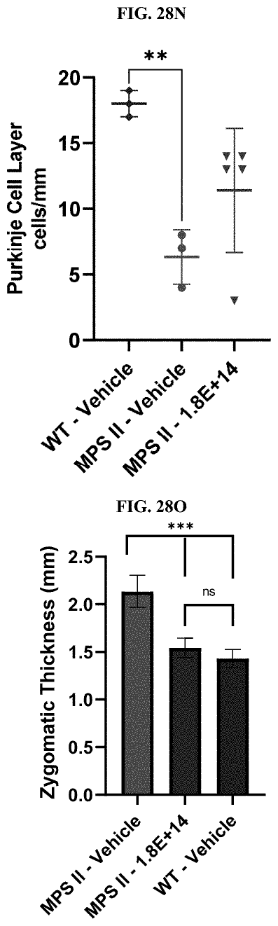

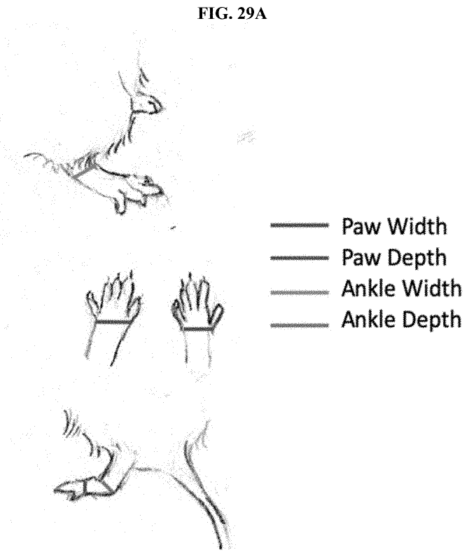

A single 1.8e14 vgs/kg dose of pHM-05217 packaged in AAVHSC15 capsid administered intravenously to MPS II mice, was found to result in significant serum I2S activity as compared to control vehicle-treated MPS II mice (FIG. 28A). Serum I2S activity was detectable out to 52 weeks post-dosing.

At 52 weeks post-dosing, vector genome and expression was maintained. The levels of vector genomes (FIG. 28B) and hIDS transcripts (FIG. 28C) in the brain, heart, liver, spleen, kidney, and lung tissue of transfer vector-treated MPS II mice were detected out to 52 weeks post-dosing. At 52 weeks post-dosing with 1.8e14 vgs/kg pHM-05217 packaged in AAVHSC15, glycosaminoglycan heparan sulfate (GAG-HS) levels in brain, heart, liver, spleen, kidney, and lung tissue were found to be reduced compared to MPS II mice treated with vehicle (FIG. 28D).

In the brain, a reduction in LAMP-1 staining was observed at 52 weeks post-dosing, as assayed by IHC in the spinal cord (FIG. 28E) and hippocampus (FIG. 28F). In the hippocampus, LAMP-1 staining was significant reduced in transfer vector-treated MPS II mice as compared to MPS II mice treated with vehicle. In FIGS. 28E and 28F, * indicates statistical significance at p≤0.05, and ns indicates not significant.

To assess the crossing of the blood-nerve barrier (BNB) and transduction of the peripheral nervous system (PNS), trigeminal ganglia were harvested from animals. Vector genomes were detected in MPS II mice administered 1.8e14 vgs/kg dose of pHM-05217 packaged in AAVHSC15 capsid at 39 weeks post-dosing, as compared to MPS II mice and wild-type mice treated with vehicle (FIG. 28G). As shown in FIG. 28G, pHM-05217 packaged in AAVHSC15 was found to cross the BNB and transduce cells of the PNS.

Liver and brain tissue specific I2S enzymatic activity was detected in MPS II mice administered 1.8e14 vgs/kg dose of pHM-05217 packaged in AAVHSC15 capsid. Liver specific I2S enzymatic activity was detected at 12, 24, 39, and 52 weeks post-dosing (FIG. 28H), and brain specific I2S enzymatic activity was detected at 12 weeks (FIG. 28I), 24 weeks (FIG. 28J), 39 weeks (FIG. 28K), and 52 weeks (FIG. 28L) post-dosing. In FIGS. 28J-28L, normal adult human brain tissue was used as an additional control.

The level of GAG-HS detected in the urine of MPS II mice administered 1.8e14 vgs/kg of pHM-05217 packaged in AAVHSC15 was found to decrease up to at least 52 weeks post-dosing, compared to MPS II mice treated with vehicle (FIG. 28M). Urine GAG-HS levels were determined by mass spectrometry and normalized to creatinine levels in each urine sample. In FIG. 28M, data is presented as average levels for each dose cohort (n=3-5 mice per group).

MPS II mice are characterized by progressive degeneration of Purkinje cell neurons in the cerebellum. Purkinje cell layer (PCL) cell linear density was quantified at 52 weeks post-dosing of MPS II mice administered 1.8e14 vgs/kg of pHM-05217 packaged in AAVHSC15. Quantitation of the Purkinje cell linear density was performed on sagittal brain sections co-stained with hematoxylin and eosin (H&E). Images of the cerebellum were collected and the total number of Purkinje cell bodies along a 400 μm long region of the Purkinje cell layer (PCL) were manually counted. Three PCL regions were randomly sampled per section (n=1 section per animal). It was found that MPS II mice administered 1.8e14 vgs/kg of pHM-05217 packaged in AAVHSC15 alleviated Purkinje cell degeneration, as compared to MPS II mice treated with vehicle (FIG. 28N). In FIG. 28N, ** indicates statistical significance at p<0.01, as calculated by a one-way analysis of variance (ANOVA) test.

MPS II mice are characterized by skeletal abnormalities including thickened zygomatic arches, thickened digits, and hind paw enlargement, as compared to wild-type animals. Zygomatic arch base morphometric measurements were assessed using a caliper on deskinned skulls of animals. MPS II mice administered 1.8e14 vgs/kg dose of pHM-05217 packaged in AAVHSC15 capsid were found to have decreased zygomatic arch thickness compared to MPS II mice treated with vehicle (FIG. 28O). In FIG. 28O, *** indicates statistical significance at p<0.01, and ns indicates not significant.

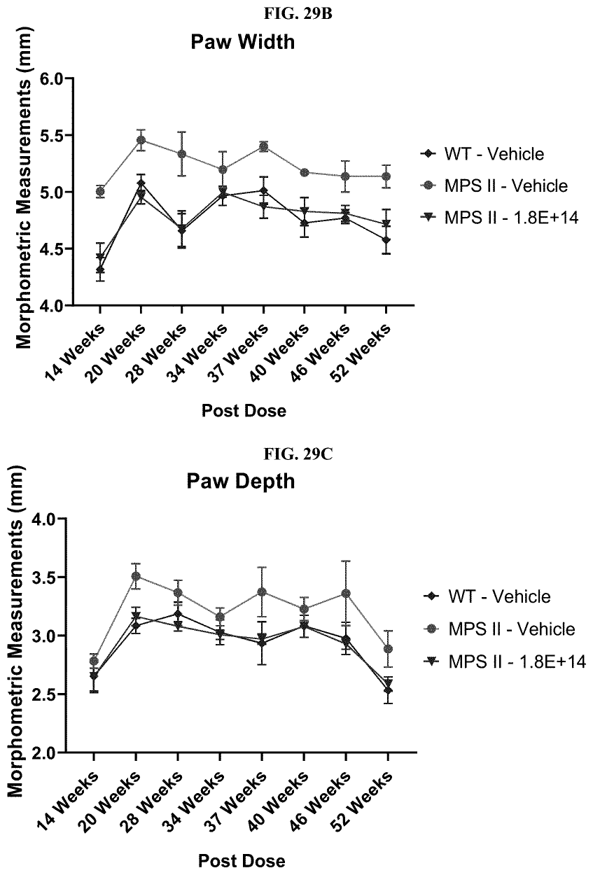

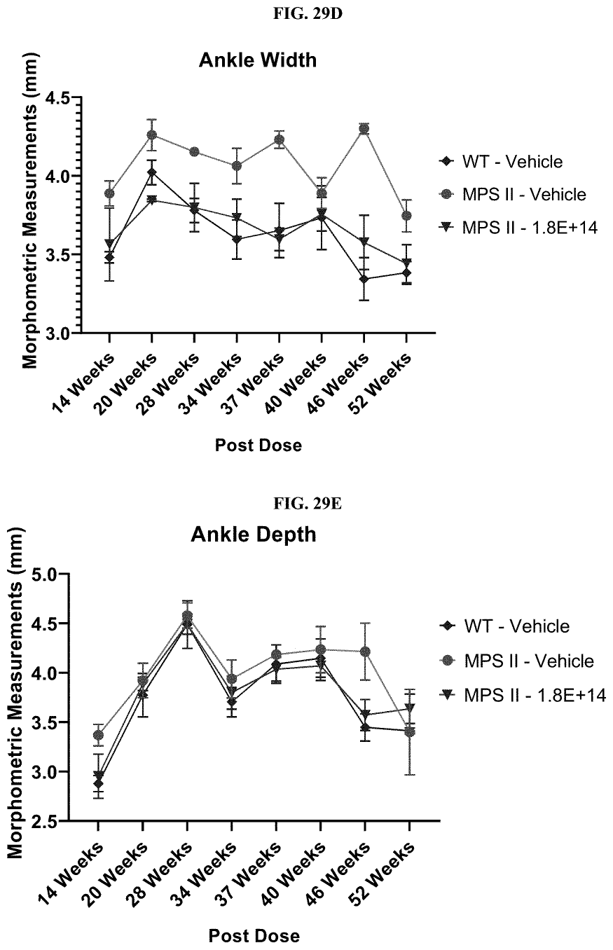

At 52 weeks post-dosing, MPS II mice treated with 1.8e14 vgs/kg of pHM-05217 packaged in AAVHSC15 display reduced hind paw and ankle enlargement compared to untreated MPS II mice (MPS II mice administered vehicle). Ankle and paw measurements were performed using a digital caliber on anesthetized mice, and according to the schematic provided in FIG. 29A. As shown in FIGS. 29B and 29C, transfer vector-treated MPS II mice exhibited ameliorated thickening of the paw, as measured by both paw width (FIG. 29B) and depth (FIG. 29C), over time, compared to vehicle-treated MPS II control mice. As shown in FIGS. 29D and 29E, transfer vector-treated MPS II mice exhibited ameliorated swelling of the hocks, as measured by both ankle width (FIG. 29D) and depth (FIG. 29E), over time, compared to vehicle-treated MPS II control mice.

### Example 10: IDS Gene Transfer in a Mucopolysaccharidosis (MPS) II (Hunter Syndrome) Mouse Model

This example describes an 8-week single-intravenous dose biological kinetics study in adult wild-type and Ids KO hemizygous mice (Ids KO hemi; also referred to as MPS II). A 1.8e14 vgs/kg dose of pHM-05217 packaged in AAVHSC15 capsid was administered intravenously to the mice, 4-5 mice per group.

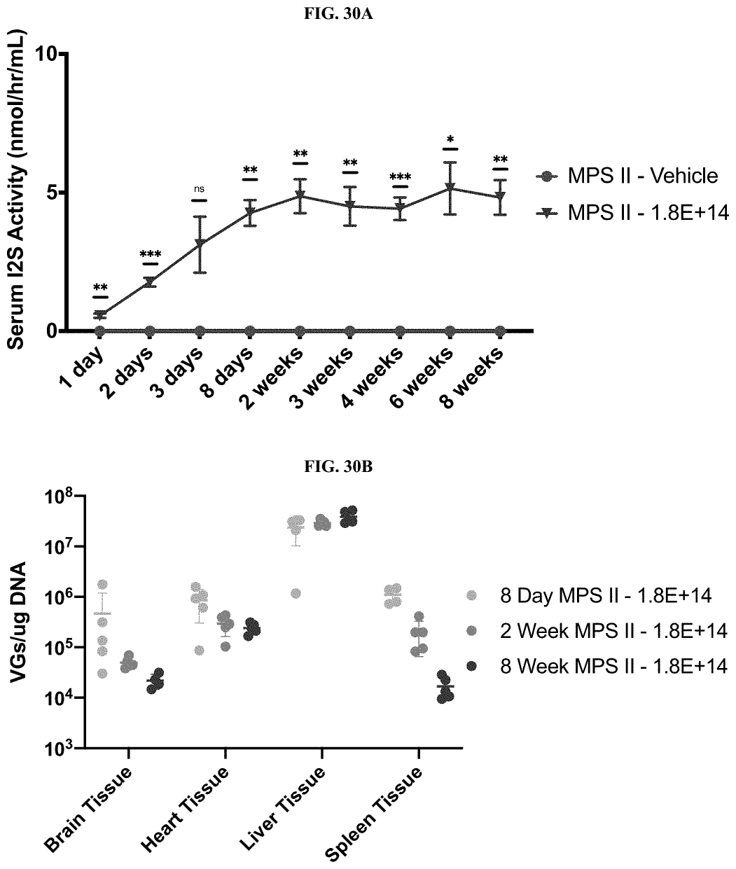

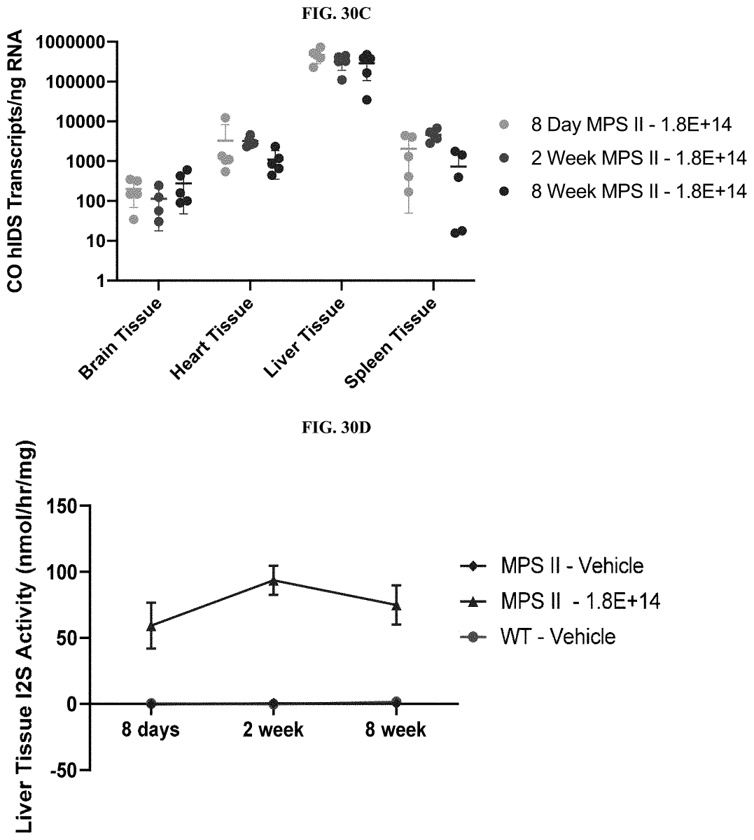

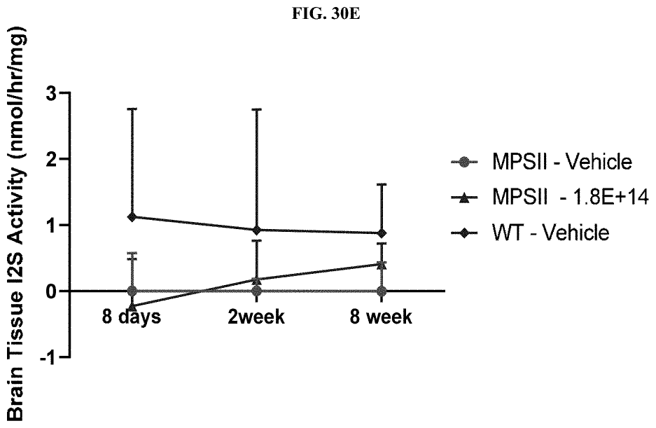

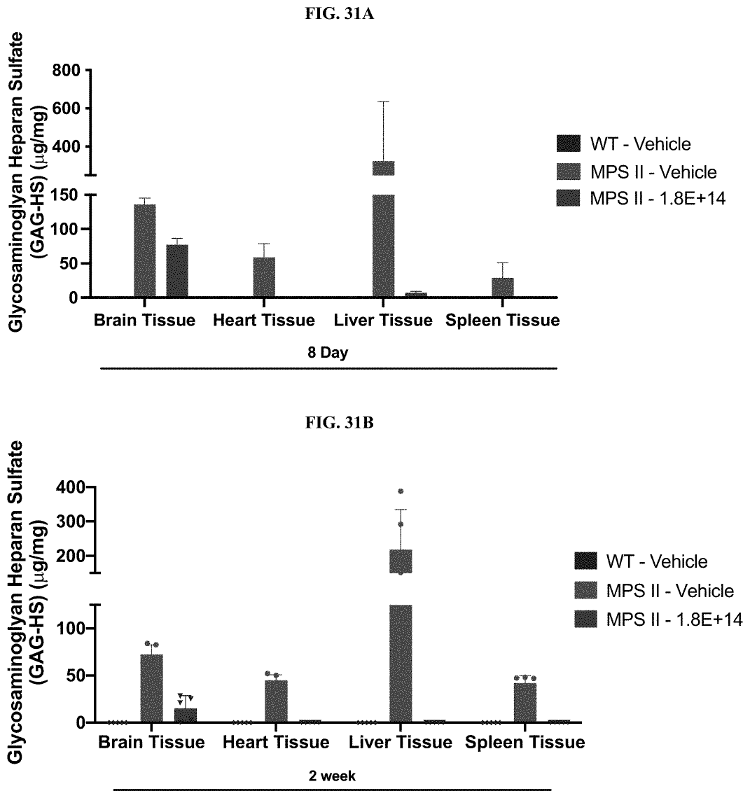

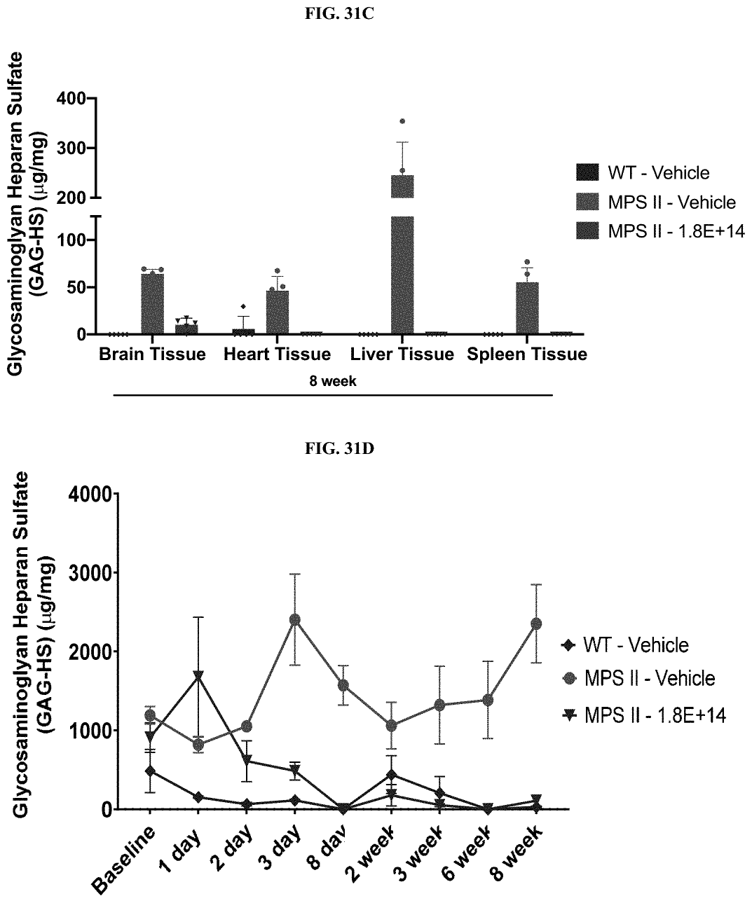

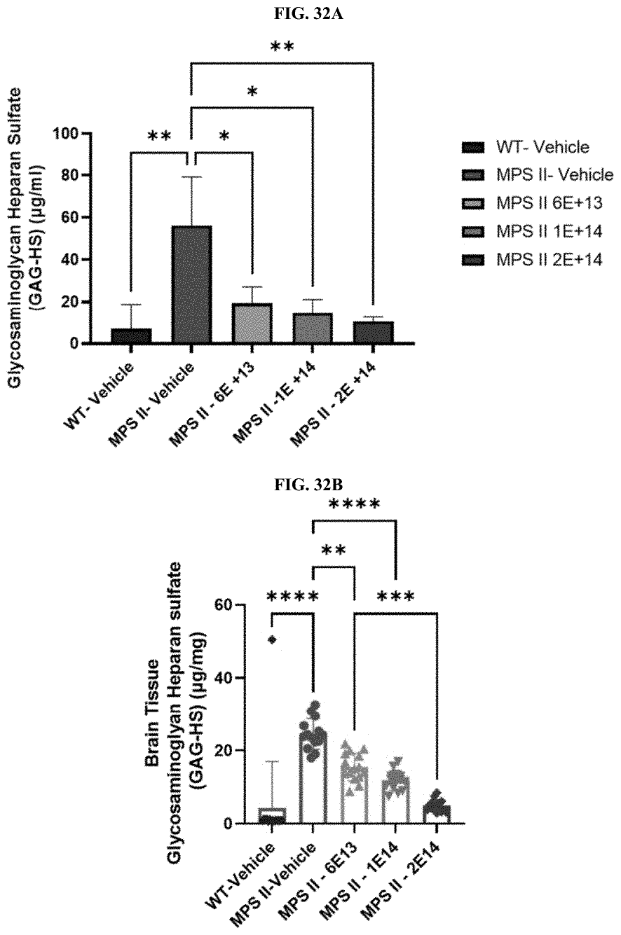

A single 1.8e14 vgs/kg dose of pHM-05217 packaged in AAVHSC15 capsid administered intravenously to MPS II mice was found to result in significant serum I2S activity, measureable as early as one day post-dosing as compared to control vehicle-treated MPS II mice (FIG. 30A). Vector genome (FIG. 30B) and expression (FIG. 30C) levels in MPS II mice intravenously administered a single 1.8e14 vgs/kg dose of pHM-05217 packaged in AAVHSC15 capsid were detected in brain, heart, liver, and spleen tissue at all tested time points. At 8 weeks post-dosing, liver tissue (FIG. 30D) and brain tissue (FIG. 30E) specific I2S activity was detected in MPS II mice intravenously administered a single 1.8e14 vgs/kg dose of pHM-05217 packaged in AAVHSC15 capsid. At the 8 day (FIG. 31A), 2 week (FIG. 31B), and 8 week (FIG. 31C) time points post-dosing with 1.8e14 vgs/kg pHM-05217 packaged in AAVHSC15, glycosaminoglycan heparan sulfate (GAG-HS) levels in brain, heart, liver, and spleen tissue were found to be reduced compared to MPS II mice treated with vehicle. The level of GAG-HS detected in the urine of MPS II mice administered 1.8e14 vgs/kg of pHM-05217 packaged in AAVHSC15 was found to decrease from baseline levels by 3 days and up to at least 8 weeks post-dosing, compared to MPS II mice treated with vehicle (FIG. 31D). In FIGS. 30A-31D, * indicates statistical significance at p≤0.05, ** indicates statistical significance at p≤0.01, *** indicates statistical significance at p≤0.001, and ns indicated no statistical significance.

### Example 11: IDS Gene Transfer in a Mucopolysaccharidosis (MPS) II (Hunter Syndrome) Mouse Model

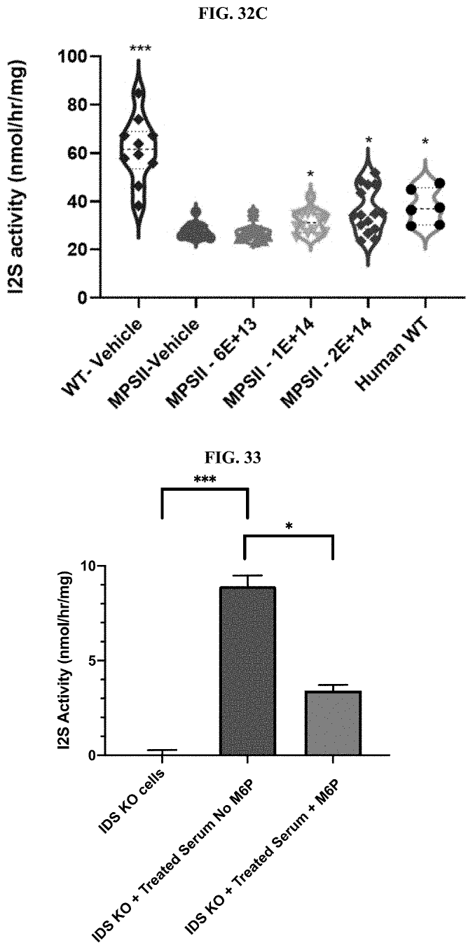

Glycosaminoglycan heparan sulfate (GAG-HS) levels in the cerebrospinal fluid (CSF) of mice were determined by measuring heparan sulfate specific disaccharides in CSF samples after heparinase digestion, using high performance liquid chromatography mass spectrometry. GAG-HS levels were measured in the CSF of wild type (WT) mice, MPS II mice treated with vehicle, and MPS II mice treated with pHM-05217 packaged in AAVHSC15 capsid administered intravenously at a dose of 6e13 vgs/kg (MPS II 6E+13), 1e14 vgs/kg (MPS II 1E+14), or 2e14 vgs/kg (MPS II 2E+14), 12 weeks post-dosing, as indicated in FIG. 32A. A reduction in CSF GAG-HS levels was observed at all doses tested, as compared to MPS II mice treated with vehicle. In FIG. 32A, each group has three CSF samples, pooled from a total of five mice. Statistical analysis was performed using a one-way analysis of variance (ANOVA). * indicates statistical significance at p<0.05, and ** indicates statistical significance at p<0.01. GAG-HS levels in the brain tissue of wild type (WT) mice, MPS II mice treated with vehicle, and MPS II mice treated with pHM-05217 packaged in AAVHSC15 capsid administered intravenously at a dose of 6e13 vgs/kg (MPS II 6E+13), 1e14 vgs/kg (MPS II 1E+14), or 2e14 vgs/kg (MPS II 2E+14), 12 weeks post-dosing, as indicated in FIG. 32B. As shown in FIG. 32B, a reduction in brain GAG-HS levels was observed at all doses tested, as compared to untreated MPS II mice treated with vehicle. Statistical analysis was performed using a one-way analysis of variance (ANOVA). **** indicates statistical significance at p<0.0001.

I2S activity was detected in the brain tissue of of wild type (WT) mice, MPS II mice, and MPS II mice treated with pHM-05217 packaged in AAVHSC15 capsid administered intravenously at a dose of 6e13 vgs/kg (MPS II 6E+13), 1e14 vgs/kg (MPS II 1E+14), or 2e14 vgs/kg (MPS II 2E+14), 12 weeks post-dosing, as indicated in FIG. 32C. Normal adut human brain tissue was used as an addition control. Statistical analysis was performed using a one-way analysis of variance (ANOVA) test. * indicates statistical significance at p<0.05, and *** indicates statistical significance at p<0.001.

### Example 12: IDS Gene Transfer Cross Correction

To investigate the cross-corrective ability of I2S expressed from an AAV gene transfer vector, 1.8e14 vgs/kg of pHM-05217 packaged in AAVHSC15 was administered intravenously to MPS II mice, and serum was assayed.

Iduronate-2-sulfatase is post-translationally modified. An initial 73-78 kDa IDS protein is converted into a 90 kDa phosphorylated precursor via the addition of a mannose 6-phosphate (M6P) moiety. The 90 kDa precursor is then processed via proteolytic cleavage through various intermediates to a major 55 kDa intermediate with the release of an 18 kDa polypeptide. Further proteolytic cleavage by a thiol protease results in the 45 kDa mature form containing hybrid and complex type oligosaccharide chains.

Briefly, IDS KO HeLa cells were cultured and incubated with mouse serum obtained from an MPS II mouse treated with 1.8e14 vgs/kg of pHM-05217 packaged in AAVHSC15, 8 days post-dosing. The cells were incubated with the treated mouse serum in the presence or absence of M6P for 48 hours. Western blots probed with a goat anti-hIDS primary antibody and detected using a donkey anti-goat secondary antibody confirmed the following: (1) hIDS protein made by pHM-05217 packaged in AAVHSC15 circulates in the serum of treated MPS II mice in the 90 kDa precursor form; (2) the 90 kDa form is catalytically active; and (3) the 90 kDa form is taken up by the IDS KO HeLa cells via an M6P-dependent pathway and processed into the intermediate 55 kDa and the mature 45 kDa protein in the lysosomes of IDS KO HeLa cells.

After incubation of IDS KO HeLa cells with mouse serum obtained from a treated MPS II mouse, the cells were centrifuged and the supernatant was removed. The pelleted cells were then lysed and assayed for hI2S activity. FIG. 33 shows the level of I2S activity detected in IDS KO cells (control), IDS KO cells incubated with treated MPS II mouse serum without M6P, and IDS KO cells incubated with treated MPS II mouse serum with M6P. As shown in FIG. 33, I2S activity was detectable in lysate of IDS KO HeLa cells treated with serum obtained from an MPS II mouse 8 days after administration of 1.8e14 vgs/kg of pHM-05217 packaged in AAVHSC15. I2S activity was found to be lower when M6P was present, suggesting, without being bound to any theory, that M6P competes for the M6P receptor and hence hI2S uptake is mediated by an M6P receptor pathway, in vitro. * indicates statistical significance at p<0.05 and *** indicates statistical significance at p<0.001.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A recombinant adeno-associated virus (rAAV) comprising:
(a) an AAV capsid comprising an AAV capsid protein; and
(b) an rAAV genome comprising a transcriptional regulatory element operably linked to an iduronate-2-sulfatase (IDS) intron-inserted coding sequence comprising an intron.

2. The rAAV of claim 1, wherein:
the IDS intron-inserted coding sequence encodes a human IDS protein;
the IDS intron-inserted coding sequence encodes the amino acid sequence set forth in SEQ ID NO: 23;
the intron is a heterologous intron; and/or
the intron has at least 85%, 86%, 87%, 88%, 89%, 90%, 91%, 92%, 93%, 94%, 95%, 96%, 97%, 98%, 99% or 100% sequence identity to the nucleotide sequence set forth in SEQ ID NO: 33.

3.-5. (canceled)

6. The rAAV of claim 1, wherein:
the intron is positioned between nucleotides in the IDS intron-inserted coding sequence that correspond to positions 708 and 709 of the IDS coding sequence set forth in SEQ ID NO: 24, optionally wherein the IDS intron-inserted coding sequence comprises a nucleotide sequence having at least 85%, 86%, 87%, 88%, 89%, 90%, 91%, 92%, 93%, 94%, 95%, 96%, 97%, 98%, 99% or 100% sequence identity to the nucleotide sequence set forth in SEQ ID NO: 25, 59, or 60;
the intron is positioned between nucleotides in the IDS intron-inserted coding sequence that correspond to positions 580 and 581 of the IDS coding sequence set forth in SEQ ID NO: 26, optionally wherein the IDS intron-inserted coding sequence comprises a nucleotide sequence having at least 85%, 86%, 87%, 88%, 89%, 90%, 91%, 92%, 93%, 94%, 95%, 96%, 97%, 98%, 99% or 100% sequence identity to the nucleotide sequence set forth in SEQ ID NO: 27; or
the IDS intron-inserted coding sequence comprises the nucleotide sequence set forth in SEQ ID NO: 25, 27, 59, or 60.

7.-10. (canceled)

11. The rAAV of claim 1, wherein:
the transcriptional regulatory element comprises one or more of the elements selected from the group consisting of a cytomegalovirus (CMV) enhancer element, cytomegalovirus (CMV) promoter, chicken-β-actin (CBA) promoter, a small chicken-β-actin (SmCBA) promoter, a glyceraldehyde 3-phosphate dehydrogenase (GAPDH) promoter, a beta-glucuronidase (GUSB) promoter, a modified human EF-1α promoter, a CALM1 promoter, a synthetic promoter, and any combination thereof;
the transcriptional regulatory element comprises a nucleotide sequence having at least 85%, 86%, 87%, 88%, 89%, 90%, 91%, 92%, 93%, 94%, 95%, 96%, 97%, 98%, 99% or 100% sequence identity to a nucleotide sequence set forth in SEQ ID NO: 29, 30, 36, 39, 40, 41, 42, 44, 46, 47, 48, or 55; and/or
the transcriptional regulatory element comprises the nucleotide sequence set forth in SEQ ID NO: 29.

12.-13. (canceled)

14. The rAAV of claim 1, wherein the rAAV genome further comprises a polyadenylation sequence 3′ to the IDS intron-inserted coding sequence, optionally wherein the polyadenylation sequence is an exogenous polyadenylation sequence, optionally wherein the exogenous polyadenylation sequence is an SV40 polyadenylation sequence, and optionally wherein the SV40 polyadenylation sequence comprises the nucleotide sequence set forth in SEQ ID NO: 34, 35, or 45.

15.-17. (canceled)

18. The rAAV of claim 1, wherein the rAAV genome comprises a nucleotide sequence set forth in SEQ ID NO: 37, 43, 52, 54, 61, 63, 65, 69, 75, or 77.

19. The rAAV of claim 1, wherein the rAAV genome further comprises a 5′ inverted terminal repeat (5′ ITR) nucleotide sequence, and a 3′ inverted terminal repeat (3′ ITR) nucleotide sequence, optionally wherein:
the 5′ ITR nucleotide sequence has at least 85%, 86%, 87%, 88%, 89%, 90%, 91%, 92%, 93%, 94%, 95%, 96%, 97%, 98%, 99% or 100% sequence identity to SEQ ID NO: 18, 20, or 49, and/or the 3′ ITR nucleotide sequence has at least 85%, 86%, 87%, 88%, 89%, 90%, 91%, 92%, 93%, 94%, 95%, 96%, 97%, 98%, 99% or 100% sequence identity to SEQ ID NO: 14, 19, 21, or 51;
the 5′ ITR nucleotide sequence has at least 85%, 86%, 87%, 88%, 89%, 90%, 91%, 92%, 93%, 94%, 95%, 96%, 97%, 98%, 99% or 100% sequence identity to SEQ ID NO: 18, and the 3′ ITR nucleotide sequence has at least 85%, 86%, 87%, 88%, 89%, 90%, 91%, 92%, 93%, 94%, 95%, 96%, 97%, 98%, 99% or 100% sequence identity to SEQ ID NO: 14;
the 5′ ITR nucleotide sequence has at least 85%, 86%, 87%, 88%, 89%, 90%, 91%, 92%, 93%, 94%, 95%, 96%, 97%, 98%, 99% or 100% sequence identity to SEQ ID NO: 18, and the 3′ ITR nucleotide sequence has at least 85%, 86%, 87%, 88%, 89%, 90%, 91%, 92%, 93%, 94%, 95%, 96%, 97%, 98%, 99% or 100% sequence identity to SEQ ID NO: 19;
the 5′ ITR nucleotide sequence has at least 85%, 86%, 87%, 88%, 89%, 90%, 91%, 92%, 93%, 94%, 95%, 96%, 97%, 98%, 99% or 100% sequence identity to SEQ ID NO: 18, and the 3′ ITR nucleotide sequence has at least 85%, 86%, 87%, 88%, 89%, 90%, 91%, 92%, 93%, 94%, 95%, 96%, 97%, 98%, 99% or 100% sequence identity to SEQ ID NO: 51;
the 5′ ITR nucleotide sequence has at least 85%, 86%, 87%, 88%, 89%, 90%, 91%, 92%, 93%, 94%, 95%, 96%, 97%, 98%, 99% or 100% sequence identity to SEQ ID NO: 49, and the 3′ ITR nucleotide sequence has at least 85%, 86%, 87%, 88%, 89%, 90%, 91%, 92%, 93%, 94%, 95%, 96%, 97%, 98%, 99% or 100% sequence identity to SEQ ID NO: 14;
the 5′ ITR nucleotide sequence has at least 85%, 86%, 87%, 88%, 89%, 90%, 91%, 92%, 93%, 94%, 95%, 96%, 97%, 98%, 99% or 100% sequence identity to SEQ ID NO: 49, and the 3′ ITR nucleotide sequence has at least 85%, 86%, 87%, 88%, 89%, 90%, 91%, 92%, 93%, 94%, 95%, 96%, 97%, 98%, 99% or 100% sequence identity to SEQ ID NO: 19;
the 5′ ITR nucleotide sequence has at least 85%, 86%, 87%, 88%, 89%, 90%, 91%, 92%, 93%, 94%, 95%, 96%, 97%, 98%, 99% or 100% sequence identity to SEQ ID NO: 49, and the 3′ ITR nucleotide sequence has at least 85%, 86%, 87%, 88%, 89%, 90%, 91%, 92%, 93%, 94%, 95%, 96%, 97%, 98%, 99% or 100% sequence identity to SEQ ID NO: 51;
the 5′ ITR nucleotide sequence has at least 85%, 86%, 87%, 88%, 89%, 90%, 91%, 92%, 93%, 94%, 95%, 96%, 97%, 98%, 99% or 100% sequence identity to SEQ ID NO: 20, and the 3′ ITR nucleotide sequence has at least 85%, 86%, 87%, 88%, 89%, 90%, 91%, 92%, 93%, 94%, 95%, 96%, 97%, 98%, 99% or 100% sequence identity to SEQ ID NO: 21; or
the 5′ ITR nucleotide sequence and the 3′ ITR nucleotide, respectively, comprise the sequences of SEQ ID NOs: 18 and 14; 18 and 19; 18 and 51; 49 and 14; 49 and 19; 49 and 51; or 20 and 21.

20.-22. (canceled)

23. The rAAV of claim 1, wherein the rAAV genome comprises a nucleotide sequence set forth in SEQ ID NO: 28, 56, 57, 71, 72, 73, or 74, or wherein the rAAV genome comprises the nucleotide sequences set forth in SEQ ID NOs: 72 and 74; 72 and 28; 73 and 74; or 73 and 28.

24. (canceled)

25. The rAAV of claim 1, wherein:
the rAAV genome is self-complementary, optionally wherein the rAAV genome comprises a nucleotide sequence set forth in SEQ ID NO: 38, 50, 62, 64, 66, 70, 76, or 78; or
the rAAV genome is single-stranded, optionally wherein the rAAV genome comprises a nucleotide sequence set forth in SEQ ID NO: 53 or 58.

26.-28. (canceled)

29. The rAAV of claim 1, wherein:
the AAV capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 1, 2, 3, 4, 5, 6, 7, 8, 9, 10, 11, 12, 13, 15, 16, or 17, optionally wherein:
(i) the amino acid in the capsid protein corresponding to amino acid 206 of SEQ ID NO: 2 is C; the amino acid in the capsid protein corresponding to amino acid 296 of SEQ ID NO: 2 is H; the amino acid in the capsid protein corresponding to amino acid 312 of SEQ ID NO: 2 is Q, the amino acid in the capsid protein corresponding to amino acid 346 of SEQ ID NO: 2 is A; the amino acid in the capsid protein corresponding to amino acid 464 of SEQ ID NO: 2 is N; the amino acid in the capsid protein corresponding to amino acid 468 of SEQ ID NO: 2 is S; the amino acid in the capsid protein corresponding to amino acid 501 of SEQ ID NO: 2 is I; the amino acid in the capsid protein corresponding to amino acid 505 of SEQ ID NO: 2 is R; the amino acid in the capsid protein corresponding to amino acid 590 of SEQ ID NO: 2 is R; the amino acid in the capsid protein corresponding to amino acid 626 of SEQ ID NO: 2 is G or Y; the amino acid in the capsid protein corresponding to amino acid 681 of SEQ ID NO: 2 is M; the amino acid in the capsid protein corresponding to amino acid 687 of SEQ ID NO: 2 is R; the amino acid in the capsid protein corresponding to amino acid 690 of SEQ ID NO: 2 is K; the amino acid in the capsid protein corresponding to amino acid 706 of SEQ ID NO: 2 is C; or, the amino acid in the capsid protein corresponding to amino acid 718 of SEQ ID NO: 2 is G;
(ii) (a) the amino acid in the capsid protein corresponding to amino acid 626 of SEQ ID NO: 2 is G, and the amino acid in the capsid protein corresponding to amino acid 718 of SEQ ID NO: 2 is G; (b) the amino acid in the capsid protein corresponding to amino acid 296 of SEQ ID NO: 2 is H, the amino acid in the capsid protein corresponding to amino acid 464 of SEQ ID NO: 2 is N, the amino acid in the capsid protein corresponding to amino acid 505 of SEQ ID NO: 2 is R, and the amino acid in the capsid protein corresponding to amino acid 681 of SEQ ID NO: 2 is M; (c) the amino acid in the capsid protein corresponding to amino acid 505 of SEQ ID NO: 2 is R, and the amino acid in the capsid protein corresponding to amino acid 687 of SEQ ID NO: 2 is R; (d) the amino acid in the capsid protein corresponding to amino acid 346 of SEQ ID NO: 2 is A, and the amino acid in the capsid protein corresponding to amino acid 505 of SEQ ID NO: 2 is R; or (e) the amino acid in the capsid protein corresponding to amino acid 501 of SEQ ID NO: 2 is I, the amino acid in the capsid protein corresponding to amino acid 505 of SEQ ID NO: 2 is R, and the amino acid in the capsid protein corresponding to amino acid 706 of SEQ ID NO: 2 is C; and/or
(iii) the capsid protein comprises the amino acid sequence of amino acids 203-736 of SEQ ID NO: 2, 3, 4, 6, 7, 10, 11, 12, 13, 15, 16, or 17;
the AAV capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 1, 2, 3, 4, 5, 6, 7, 8, 9, 10, 11, 12, 13, 15, 16, or 17, optionally wherein:
(i) the amino acid in the capsid protein corresponding to amino acid 151 of SEQ ID NO: 2 is R; the amino acid in the capsid protein corresponding to amino acid 160 of SEQ ID NO: 2 is D; the amino acid in the capsid protein corresponding to amino acid 206 of SEQ ID NO: 2 is C; the amino acid in the capsid protein corresponding to amino acid 296 of SEQ ID NO: 2 is H; the amino acid in the capsid protein corresponding to amino acid 312 of SEQ ID NO: 2 is Q; the amino acid in the capsid protein corresponding to amino acid 346 of SEQ ID NO: 2 is A; the amino acid in the capsid protein corresponding to amino acid 464 of SEQ ID NO: 2 is N; the amino acid in the capsid protein corresponding to amino acid 468 of SEQ ID NO: 2 is S; the amino acid in the capsid protein corresponding to amino acid 501 of SEQ ID NO: 2 is I; the amino acid in the capsid protein corresponding to amino acid 505 of SEQ ID NO: 2 is R; the amino acid in the capsid protein corresponding to amino acid 590 of SEQ ID NO: 2 is R; the amino acid in the capsid protein corresponding to amino acid 626 of SEQ ID NO: 2 is G or Y; the amino acid in the capsid protein corresponding to amino acid 681 of SEQ ID NO: 2 is M; the amino acid in the capsid protein corresponding to amino acid 687 of SEQ ID NO: 2 is R; the amino acid in the capsid protein corresponding to amino acid 690 of SEQ ID NO: 2 is K; the amino acid in the capsid protein corresponding to amino acid 706 of SEQ ID NO: 2 is C; or, the amino acid in the capsid protein corresponding to amino acid 718 of SEQ ID NO: 2 is G;
(ii) (a) the amino acid in the capsid protein corresponding to amino acid 626 of SEQ ID NO: 2 is G, and the amino acid in the capsid protein corresponding to amino acid 718 of SEQ ID NO: 2 is G; (b) the amino acid in the capsid protein corresponding to amino acid 296 of SEQ ID NO: 2 is H, the amino acid in the capsid protein corresponding to amino acid 464 of SEQ ID NO: 2 is N, the amino acid in the capsid protein corresponding to amino acid 505 of SEQ ID NO: 2 is R, and the amino acid in the capsid protein corresponding to amino acid 681 of SEQ ID NO: 2 is M; (c) the amino acid in the capsid protein corresponding to amino acid 505 of SEQ ID NO: 2 is R, and the amino acid in the capsid protein corresponding to amino acid 687 of SEQ ID NO: 2 is R; (d) the amino acid in the capsid protein corresponding to amino acid 346 of SEQ ID NO: 2 is A, and the amino acid in the capsid protein corresponding to amino acid 505 of SEQ ID NO: 2 is R; or (e) the amino acid in the capsid protein corresponding to amino acid 501 of SEQ ID NO: 2 is I, the amino acid in the capsid protein corresponding to amino acid 505 of SEQ ID NO: 2 is R, and the amino acid in the capsid protein corresponding to amino acid 706 of SEQ ID NO: 2 is C; and/or
(iii) the capsid protein comprises the amino acid sequence of amino acids 138-736 of SEQ ID NO: 2, 3, 4, 5, 6, 7, 9, 10, 11, 12, 13, 15, 16, or 17; and/or
the AAV capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 1, 2, 3, 4, 5, 6, 7, 8, 9, 10, 11, 12, 13, 15, 16, or 17, optionally wherein:
(i) the amino acid in the capsid protein corresponding to amino acid 2 of SEQ ID NO: 2 is T; the amino acid in the capsid protein corresponding to amino acid 65 of SEQ ID NO: 2 is I; the amino acid in the capsid protein corresponding to amino acid 68 of SEQ ID NO: 2 is V; the amino acid in the capsid protein corresponding to amino acid 77 of SEQ ID NO: 2 is R; the amino acid in the capsid protein corresponding to amino acid 119 of SEQ ID NO: 2 is L; the amino acid in the capsid protein corresponding to amino acid 151 of SEQ ID NO: 2 is R; the amino acid in the capsid protein corresponding to amino acid 160 of SEQ ID NO: 2 is D; the amino acid in the capsid protein corresponding to amino acid 206 of SEQ ID NO: 2 is C; the amino acid in the capsid protein corresponding to amino acid 296 of SEQ ID NO: 2 is H; the amino acid in the capsid protein corresponding to amino acid 312 of SEQ ID NO: 2 is Q; the amino acid in the capsid protein corresponding to amino acid 346 of SEQ ID NO: 2 is A; the amino acid in the capsid protein corresponding to amino acid 464 of SEQ ID NO: 2 is N; the amino acid in the capsid protein corresponding to amino acid 468 of SEQ ID NO: 2 is S; the amino acid in the capsid protein corresponding to amino acid 501 of SEQ ID NO: 2 is I; the amino acid in the capsid protein corresponding to amino acid 505 of SEQ ID NO: 2 is R; the amino acid in the capsid protein corresponding to amino acid 590 of SEQ ID NO: 2 is R; the amino acid in the capsid protein corresponding to amino acid 626 of SEQ ID NO: 2 is G or Y; the amino acid in the capsid protein corresponding to amino acid 681 of SEQ ID NO: 2 is M; the amino acid in the capsid protein corresponding to amino acid 687 of SEQ ID NO: 2 is R; the amino acid in the capsid protein corresponding to amino acid 690 of SEQ ID NO: 2 is K; the amino acid in the capsid protein corresponding to amino acid 706 of SEQ ID NO: 2 is C; or, the amino acid in the capsid protein corresponding to amino acid 718 of SEQ ID NO: 2 is G;
(ii) (a) the amino acid in the capsid protein corresponding to amino acid 2 of SEQ ID NO: 2 is T, and the amino acid in the capsid protein corresponding to amino acid 312 of SEQ ID NO: 2 is Q; (b) the amino acid in the capsid protein corresponding to amino acid 65 of SEQ ID NO: 2 is I, and the amino acid in the capsid protein corresponding to amino acid 626 of SEQ ID NO: 2 is Y; (c) the amino acid in the capsid protein corresponding to amino acid 77 of SEQ ID NO: 2 is R, and the amino acid in the capsid protein corresponding to amino acid 690 of SEQ ID NO: 2 is K; (d) the amino acid in the capsid protein corresponding to amino acid 119 of SEQ ID NO: 2 is L, and the amino acid in the capsid protein corresponding to amino acid 468 of SEQ ID NO: 2 is S; (e) the amino acid in the capsid protein corresponding to amino acid 626 of SEQ ID NO: 2 is G, and the amino acid in the capsid protein corresponding to amino acid 718 of SEQ ID NO: 2 is G; (f) the amino acid in the capsid protein corresponding to amino acid 296 of SEQ ID NO: 2 is H, the amino acid in the capsid protein corresponding to amino acid 464 of SEQ ID NO: 2 is N, the amino acid in the capsid protein corresponding to amino acid 505 of SEQ ID NO: 2 is R, and the amino acid in the capsid protein corresponding to amino acid 681 of SEQ ID NO: 2 is M; (g) the amino acid in the capsid protein corresponding to amino acid 505 of SEQ ID NO: 2 is R, and the amino acid in the capsid protein corresponding to amino acid 687 of SEQ ID NO: 2 is R; (h) the amino acid in the capsid protein corresponding to amino acid 346 of SEQ ID NO: 2 is A, and the amino acid in the capsid protein corresponding to amino acid 505 of SEQ ID NO: 2 is R; or (i) the amino acid in the capsid protein corresponding to amino acid 501 of SEQ ID NO: 2 is I, the amino acid in the capsid protein corresponding to amino acid 505 of SEQ ID NO: 2 is R, and the amino acid in the capsid protein corresponding to amino acid 706 of SEQ ID NO: 2 is C; and/or
(iii) the capsid protein comprises the amino acid sequence of amino acids 1-736 of SEQ ID NO: 2, 3, 4, 5, 6, 7, 8, 9, 10, 11, 12, 13, 15, 16, or 17.

30.-40. (canceled)

41. A method for expressing an iduronate-2-sulfatase (IDS) polypeptide in a cell, the method comprising transducing the cell with a recombinant adeno-associated virus (rAAV) of claim 1.

42. The method of claim 41, wherein:
the cell is a cell of the central nervous system;
the cell is a cell of the central nervous system region selected from the group consisting of the spinal cord, the motor cortex, the sensory cortex, the hippocampus, the putamen, the cerebellum optionally the cerebellar nuclei, and any combination thereof;
the cell is a neuron or a glial cell, optionally wherein the cell is a neuron or a glial cell of the central nervous system or the peripheral nervous system;
the cell is a cell selected from the group consisting of a motor neuron, an astrocyte, an oligodendrocyte, a cell of the cerebral cortex in the central nervous system, a sensory neuron of the peripheral nervous system, a Schwann cell, and any combination thereof;
the cell is a cell of the liver, the heart, the lung, the kidney, or the spleen; and/or
the cell is in a mammalian subject and the rAAV is administered to the subject in an amount effective to transduce the cell in the subject.

43.-47. (canceled)

48. A pharmaceutical composition comprising the rAAV of claim 1.

49. A method for treating a subject having Hunter Syndrome (HS), the method comprising administering to the subject an effective amount of the rAAV of claim 1.

50. The method of claim 49, wherein the rAAV or pharmaceutical composition is administered intravenously, optionally wherein Hunter Syndrome (HS) is associated with an iduronate-2-sulfatase (IDS) gene mutation, and optionally wherein the subject is a human subject.

51.-52. (canceled)

53. A packaging system for preparation of an rAAV, wherein the packaging system comprises:
(a) a first nucleotide sequence encoding one or more AAV Rep proteins;
(b) a second nucleotide sequence encoding an AAV capsid protein; and
(c) a third nucleotide sequence comprising the rAAV genome sequence of the rAAV of claim 1.

54.-58. (canceled)

59. A method for recombinant preparation of an rAAV, the method comprising introducing the packaging system of claim 53 into a cell under conditions whereby the rAAV is produced.

60. A polynucleotide comprising a nucleic acid sequence having at least 85%, 86%, 87%, 88%, 89%, 90%, 91%, 92%, 93%, 94%, 95%, 96%, 97%, 98%, 99% or 100% sequence identity to the nucleotide sequence set forth in SEQ ID NO: 25, 26, 27, 37, 38, 43, 50, 52, 53, 54, 58, 59, 60, 61, 62, 63, 64, 65, 66, 67, 68, 69, 70, 75, 76, 77, or 78, optionally wherein the polynucleotide is comprised within a viral vector or plasmid vector.

61. A recombinant cell comprising the polynucleotide of claim 60.

62.-64. (canceled)

65. A method for treating a subject having Hunter Syndrome (HS), the method comprising administering to the subject an effective amount of the pharmaceutical composition of claim 48.
